(19) United States
(12) Patent Application Publication
(10) Pub. No.: US 2024/0374518 A1
(43) Pub. Date: Nov. 14, 2024

(54) AN ANTI-ANGIOGENIC AGENT AND RELATED METHODS

(71) Applicants: Xinyi Su, Singapore (SG); Xinxin Zhao, Singapore (SG); Queenie Shu Woon Tan, Singapore (SG); Walter Hunziker, Singapore (SG); Zengping Liu, Singapore (SG); Xian Jun Loh, Singapore (SG); Kun Xue, Singapore (SG); Veluchamy Amutha Barathi, Singapore (SG)

(72) Inventors: Xinyi Su, Singapore (SG); Xinxin Zhao, Singapore (SG); Queenie Shu Woon Tan, Singapore (SG); Walter Hunziker, Singapore (SG); Zengping Liu, Singapore (SG); Xian Jun Loh, Singapore (SG); Kun Xue, Singapore (SG); Veluchamy Amutha Barathi, Singapore (SG)

(21) Appl. No.: 18/580,830

(22) PCT Filed: Jul. 7, 2022
(86) PCT No.: PCT/SG2022/050473

§ 371 Date: Jan. 19, 2024

(30) Foreign Application Priority Data
Jul. 26, 2021 (SG) 10202108149Q
Oct. 29, 2021 (SG) 10202112107V

(51) Int. Cl. A61K 9/107; A61K 38/17; A61K 39/00; A61K 47/34; A61P 27/02; C07K 16/22

(52) U.S. Cl. CPC A61K 9/1075; A61K 38/179; A61K 47/34; A61P 27/02; C07K 16/22; A61K 2039/505; C07K 2317/24

(57) ABSTRACT

There is provided an anti-angiogenic agent comprising: a multi-block copolymer in the form of one or more micelles, wherein the copolymer comprises a first poly (alkylene glycol) block, a second poly (alkylene glycol) block and a polyester block. There is also provided a method of preparing said anti-angiogenic agent and medical uses of said anti-angiogenic agent.

### TECHNICAL FIELD

The present disclosure relates broadly to an anti-angiogenic agent. The present disclosure also relates to a method of preparing the anti-angiogenic agent and related uses.

### BACKGROUND

Retinal diseases, such as neovascular age-related macular degeneration (nAMD) and diabetic macular oedema (DMO) contribute to a significant proportion of visual impairment globally. The current first-line treatment for these sight-threatening retinal diseases is the intravitreal injection (IVT) of anti-vascular endothelial growth factor (anti-VEGF) compounds to prevent the growth of abnormal blood vessels and leakage of fluid from these vessels. Food and Drug Administration (FDA) approved anti-VEGF compounds include ranibizumab (Lucentis®, Novartis) and aflibercept (Eylea®, Bayer). Bevacizumab (Avastin®, Novartis) is also used off-label for the treatment of such diseases, with similar efficacy. These anti-VEGF compounds are protein-based biologics.

As patients require multiple IVTs for disease control, it poses a tremendous burden on the individual and the healthcare system. Furthermore, the invasive nature of IVTs can result in sight-threatening complications such as endophthalmitis. As long-term treatment regimens have poor compliance rates, there has been significant interest in creating a more tolerable therapeutic strategy. In recent years, to reduce the number of IVTs a patient has to undergo, pharmaceutical companies have developed compounds with intrinsically longer lasting anti-angiogenic effects or sustained drug delivery systems. In 2019, the FDA approved brolucizumab (Beovu®, Novartis) which is capable of sustaining a longer effect as compared to previous stated compounds. However, such strategies, still do not completely eliminate IVTs but serve only to reduce the number of IVTs a patient has to undergo.

Topical delivery of anti-VEGFs to the posterior segment will avoid the above stated complications. However, multiple static and dynamic ocular barriers between the cornea and the retina, prevent drugs from attaining a therapeutic concentration at the retina sufficient for disease control. While topical drug delivery systems for small molecule drugs have been met with initial success, developing such systems for the FDA-approved, protein-based, anti-VEGF compounds has been challenging. Hence, different groups have attempted topical delivery of small molecules with anti-angiogenic effects instead with limited efficacy against the disease. Majority of these successful topical drug delivery systems have been for the delivery of small molecules to the anterior segment of the eye, not the retina.

To date, the only anti-angiogenic eyedrop in clinical trials is PAN-90806 (PanOptica), a small molecule tyrosine-kinase inhibitor (TKI) against VEGF-receptor 2 for nAMD treatment. PAN-90806 is a product under development. Other pre-clinical studies have demonstrated the use of a hydrogel-based drug delivery system to prolong cornea residence time for delivery of hydrophobic small molecules to the retina but have not demonstrated their use to enhance penetration of hydrophilic protein-based drugs across ocular barriers for drug delivery.

Very few studies have reported the use of drug delivery platforms for the topical delivery of hydrophilic macromolecular biologics for retinal therapy. For example, liposomes with additional anionic phospholipid binding protein Annexin A5 (Anx5) have been used to enhance the delivery of bevacizumab to the rabbit vitreous. Addition of AnxA5 significantly increased bevacizumab concentration in both the vitreous of the rat eye and rabbit eye, but therapeutic efficacy was not demonstrated in this study. In addition, cell-penetrating peptides composed of oligo arginine have also been used to successfully deliver bevacizumab to the porcine vitreous, showing therapeutic effect in a model of choroidal neovascularisation (CNV). While cell-penetrating peptides have been widely used for intracellular delivery, it has not been approved by the FDA due to lingering concerns of stability and immunogenicity.

In view of the above, there is a need to address or at least ameliorate the above-mentioned problems. In particular, there is a need for an anti-angiogenic agent, a method of preparing the anti-angiogenic agent and related uses of the anti-angiogenic agent that address or at least ameliorate the above-mentioned problems.

### SUMMARY

In one aspect, there is provided an anti-angiogenic agent comprising:
a multi-block copolymer in the form of one or more micelles, wherein the copolymer comprises a first poly(alkylene glycol) block, a second poly(alkylene glycol) block and a polyester block.

In one embodiment, the copolymer comprises at least urethane/carbamate linkage(s) and/or allophanate linkage(s).

In one embodiment, the molar ratio of the first poly(alkylene glycol) block to the second poly(alkylene glycol) block to the polyester block in the copolymer is about 1 to 10:1:0.01 to 1.5.

In one embodiment, the first and second poly(alkylene glycol) are selected from the group consisting of poly(ethylene glycol) (PEG), poly(propylene glycol) (PPG), poly(butylene glycol) and combinations thereof; and the polyester is selected from the group consisting of polycaprolactone (PCL), poly(lactic acid) (PLA), poly(lactic-co-glycolic acid) (PLGA), polyhydroxyalkanoate (PHA) and combinations thereof.

In one embodiment, the total polymer concentration of the copolymer is in the range of from 0.01 wt % to 6 wt %.

In one embodiment, the anti-angiogenic agent comprises a water content of at least 90 wt %.

In one embodiment, the one or more micelles have a hydrodynamic size of from 1 nm to 100 nm.

In one embodiment, the anti-angiogenic agent further comprises one or more bioactive(s) complexed with or encapsulated by the copolymer micelles.

In one embodiment, the one or more bioactive(s) comprises an anti-vascular endothelial growth factor (anti-VEGF).

In one embodiment, the anti-VEGF is selected from the group consisting of bevacizumab, aflibercept, ranibizumab and brolucizumab.

In one embodiment, the one or more bioactive(s) is encapsulated by the copolymer micelles at an encapsulation efficiency of more than 25%.

In one embodiment, the anti-angiogenic agent is formulated as a topical ophthalmic formulation.

In one aspect, there is provided a method of preparing anti-angiogenic agent as disclosed herein, the method comprising:
adding a copolymer to an aqueous medium at a concentration that is no less than the critical micelle concentration of the copolymer but no more than the sol-gel transition concentration of the copolymer, to form micelles,
wherein the copolymer comprises a first poly(alkylene glycol) block, a second poly(alkylene glycol) block and a polyester block.

In one embodiment, the concentration of the copolymer in the aqueous medium is in the range of from 0.01 wt % to 6 wt %.

In one embodiment, the method further comprises complexing or encapsulating one or more bioactive(s) with the micelle.

In one embodiment, the method further comprises coupling the first poly(alkylene glycol) block, the second poly(alkylene glycol) block and the polyester block together by at least urethane/carbamate linkage(s) and/or allophanate linkage(s).

In one embodiment, the first and second poly(alkylene glycol) are selected from the group consisting of poly(ethylene glycol) (PEG), poly(propylene glycol) (PPG), poly(butylene glycol) and combinations thereof; and the polyester is selected from the group consisting of polycaprolactone (PCL), poly(lactic acid) (PLA), poly(lactic-co-glycolic acid) (PLGA), polyhydroxyalkanoate (PHA) and combinations thereof.

In one embodiment, the coupling step is carried out in the presence of a coupling agent comprising an isocyanate monomer that contains two isocyanate functional groups.

In one embodiment, the coupling step is carried out in the presence of a catalyst selected from the group consisting of alkyltin compounds, aryltin compounds and dialkyltin diesters such as dibutyltin dilaurate, dibutyltin diacetate, dibutyltin dioctanoate and dibutyltin distearate.

In one embodiment, the coupling step is carried out in the presence of a solvent selected from the group consisting of toluene, benzene, xylene, halogenated organic solvents, halogenated alkane solvents, chlorinated solvents, dichloromethane, dichloroethane, tetrachloromethane and chloroform (or trichloromethane).

In one aspect, there is provided an anti-angiogenic agent as disclosed herein for use in medicine.

In one aspect, there is provided an anti-angiogenic agent as disclosed herein for use in the prophylaxis or treatment of an eye disorder.

In one aspect, there is provided an anti-angiogenic agent as disclosed herein for use in the prophylaxis or treatment of cancer.

In one aspect, there is provided use of an anti-angiogenic agent as disclosed herein in the manufacture of a medicament for the prophylaxis or treatment of an eye disorder.

In one aspect, there is provided use of an anti-angiogenic agent as disclosed herein in the manufacture of a medicament for the prophylaxis or treatment of cancer.

In one aspect, there is provided a method of preventing or treating an eye disorder, the method comprising administering the anti-angiogenic agent as disclosed herein to a subject in need thereof.

In one aspect, there is provided a method of preventing or treating cancer, the method comprising administering the anti-angiogenic agent as disclosed herein to a subject in need thereof.

In one embodiment, the eye disorder is selected from the group consisting of angiogenic eye disorders, ocular diseases in the anterior segment, ocular diseases in the posterior segment, neovascular related ophthalmic posterior segment diseases, retinal diseases, neovascular age-related macular degeneration (AMD) such as neovascular AMD, diabetic retinopathies, diabetic macular oedema (DMO), choroidal neovascularisation (CNV), central retinal vein occlusion (CRVO), corneal neovascularization, and retinal neovascularization.

In one embodiment, the anti-angiogenic agent is to be topically administered to a subject in need thereof.

In one embodiment, the anti-angiogenic agent is formulated as an eye drop.

### Definitions

The term “polymer” as used herein refers to a chemical compound comprising repeating units and is created through a process of polymerization. The units composing the polymer are typically derived from monomers and/or macromonomers. A polymer typically comprises repetition of a number of constitutional units.

The terms “monomer” or “macromonomer” as used herein refer to a chemical entity that may be covalently linked to one or more of such entities to form a polymer.

The term “bond” refers to a linkage between atoms in a compound or molecule. The bond may be a single bond, a double bond, or a triple bond.

In the definitions of a number of substituents below, it is stated that “the group may be a terminal group or a bridging group”. This is intended to signify that the use of the term is intended to encompass the situation where the group is a terminal group/moiety as well as the situation where the group is a linker between two other portions of the molecule. Using the term “alkyl” having 1 carbon atom as an example, it will be appreciated that when existing as a terminal group, the term “alkyl” having 1 carbon atom may mean —CH₃ and when existing as a bridging group, the term “alkyl” having 1 carbon atom may mean —CH₂— or the like.

The term “alkyl” as a group or part of a group refers to a straight or branched aliphatic hydrocarbon group having 1 to 20 carbon atoms, 1 to 10 carbon atoms, 1 to 6 carbon atoms, or 1, 2, 3, 4, 5, 6, 7, 8, 9, 10, 11, 12, 13, 14, 15, 16, 17, 18, 19 or 20 carbon atoms. Examples of suitable straight and branched alkyl substituents include methyl, ethyl, n-propyl, 2-propyl, isopropyl, n-butyl, isobutyl, sec-butyl, t-butyl, hexyl, amyl, 1,2-dimethylpropyl, 1,1-dimethylpropyl, pentyl, isopentyl, hexyl, 4-methylpentyl, 1-methylpentyl, 2-methylpentyl, 3-methylpentyl, 2,2-dimethylbutyl, 3,3-dimethylbutyl, 1,2-dimethylbutyl, 1,3-dimethylbutyl, 1,2,2-trimethylpropyl, 1,1,2-trimethylpropyl, 2-ethylpentyl, 3-ethylpentyl, heptyl, 1-methylhexyl, 2,2-dimethylpentyl, 3,3-dimethylpentyl, 4,4-dimethylpentyl, 1,2-dimethylpentyl, 1,3-dimethylpentyl, 1,4-dimethylpentyl, 1,2,3-trimethylbutyl, 1,1,2-trimethylbutyl, 1,1,3-trimethylbutyl, 5-methylheptyl, 1-methylheptyl, octyl, nonyl, decyl and the like. The group may be a terminal group or a bridging group.

The term “alkenyl” as a group or part of a group denotes an aliphatic hydrocarbon group containing at least one carbon-carbon double bond and which may be straight or branched having 2 to 20 carbon atoms, 2 to 10 carbon atoms, 2 to 6 carbon atoms, or 2, 3, 4, 5, 6, 7, 8, 9, 10, 11, 12, 13, 14, 15, 16, 17, 18, 19 or 20 carbon atoms in the chain. The group may contain a plurality of double bonds and the orientation about each double bond is independently E or Z.

Exemplary alkenyl groups include, but are not limited to, ethenyl, vinyl, allyl, 1-methylvinyl, 1-propenyl, 2-propenyl, 2-methyl-1-propenyl, 2-methyl-1-propenyl, 1-butenyl, 2-butenyl, 3-butentyl, 1,3-butadienyl, 1-pentenyl, 2-pententyl, 3-pentenyl, 4-pentenyl, 1,3-pentadienyl, 2,4-pentadienyl, 1,4-pentadienyl, 3-methyl-2-butenyl, 1-hexenyl, 2-hexenyl, 3-hexenyl, 1,3-hexadienyl, 1,4-hexadienyl, 2-methylpentenyl, 1-heptenyl, 2-heptentyl, 3-heptenyl, 1-octenyl, 2-octenyl, 3-octenyl, 1-nonenyl, 2-nonenyl, 3-nonenyl, 1-decenyl, 2-decenyl, 3-decenyl and the like. The group may be a terminal group or a bridging group.

The term “alkynyl” as a group or part of a group denotes an aliphatic hydrocarbon group containing at least one carbon-carbon triple bond and which may be straight or branched having 2 to 20 carbon atoms, 2 to 10 carbon atoms, 2 to 6 carbon atoms, or 2, 3, 4, 5, 6, 7, 8, 9, 10, 11, 12, 13, 14, 15, 16, 17, 18, 19 or 20 carbon atoms in the chain. The group may contain a plurality of triple bonds. Exemplary alkynyl groups include, but are not limited to, acetylenyl, propynyl, 1-butynyl, 2-butynyl, 3-butynyl, 1-pentynyl, 2-pentynyl, 3-methyl-1-butynyl, 4-pentynyl, 1-hexynyl, 2-hexynyl, 5-hexynyl, 1-heptynyl, 2-heptynyl, 6-heptynyl, 1-octynyl, 2-octynyl, 7-octynyl, 1-nonynyl, 2-nonynyl, 8-nonynyl, 1-decynyl, 2-decynyl, 9-decynyl and the like. The group may be a terminal group or a bridging group.

The term “alkylene” as used herein is intended to broadly refer to an aliphatic hydrocarbon group (e.g., alkyl, alkenyl or alkynyl as defined herein) that is divalent. The alkylene groups may be linear, branched, saturated, unsaturated, cyclic, acyclic, substituted and/or unsubstituted. Examples of alkylene include methylene (i.e. —CH₂— or “alkylene” having 1 carbon atom), ethylene (i.e. —CH₂CH₂— or “alkylene” having 2 carbon atoms), propylene (i.e. “alkylene” having 3 carbon atoms) and the like.

The term “poly(alkylene glycol)” as used herein is intended to broadly refer to a polymer containing an ether group (i.e. —O—R—, where R is alkylene as defined herein) in a repeating unit. In various embodiments, the terms poly(alkylene glycol) may be used interchangeably with the terms “polyglycol”, “polyether” or “poly(alkylene oxide)”. Examples of poly(alkylene glycol) include poly(ethylene glycol) (PEG) (or polyethylene oxide), poly(propylene glycol) (PPG) (or polypropylene oxide), poly(butylene glycol) (or polybutylene oxide) and the like.

The term “polyester” as used herein is intended to broadly refer to a polymer containing an ester group (i.e. —O—C(═O)—) in a repeating unit. Examples of polyester include polycaprolactone (PCL), poly(lactic acid) or polylactide (PLA), polyglycolic acid (PGA), polyethylene adipate diol (PEA), poly(ethylene terephthalate) (PET), polyhydroxyalkanoate (PHA) (e.g., polyhydroxybutyrate (PHB)), poly(lactic-co-glycolic acid) (PLGA) and the like.

The terms “urethane linkage” and “carbamate linkage” as used herein are intended to broadly refer to a group containing —O—C(═O)—N(R)—, where R is a hydrogen or an organic group (e.g., hydrocarbon group). For example, the “urethane linkage” or “carbamate linkage” may be —O—(C═O)—N(H)—. In various embodiments, the “urethane linkage” or “carbamate linkage” may contain 1, 2, 3, 4, 5, 6, 7, 8, 9, 10 or more than 10 urethane/carbamate groups. For example, the “urethane linkage” or “carbamate linkage” may also be —O—(C═O)—N(R)—R′—N(R)—(C═O)—O— or —O—(C═O)—N(H)—R′—N(H)—(C═O)—O—, where R and R′ are each independently a hydrogen or an organic group (e.g., hydrocarbon group).

The term “allophanate linkage” as used herein is intended to broadly refer to a group containing [—N(R)—C(═O)]—N(R′)—C(═O)—O—, where R and R′ are each independently a hydrogen or an organic group (e.g., hydrocarbon group). In various embodiments, the “allophanate linkage” refers to a group that is formed by the reaction between an isocyanate group and an urethane group, where R and R′ are derived from the isocyanate and urethane respectively. It will be appreciated that in various embodiments, the formation of allophanate is reversible. In various embodiments, the “allophanate linkage” may contain 1, 2, 3, 4, 5, 6, 7, 8, 9, 10 or more than 10 allophanate groups.

The term “carbonate linkage” as used herein is intended to broadly refer to a group containing —O—C(═O)—O—. In various embodiments, the “carbonate linkage” may contain 1, 2, 3, 4, 5, 6, 7, 8, 9, 10 or more than 10 carbonate groups.

The term “ester linkage” as used herein is intended to broadly refer to a group containing —O—C(═O)—. In various embodiments, the “ester linkage” may contain 1, 2, 3, 4, 5, 6, 7, 8, 9, 10 or more than 10 ester groups.

The term “urea linkage” as used herein is intended to broadly refer to a group containing —N(R)—C(═O)—N(R′)—, where R and R′ are each independently a hydrogen or an organic group (e.g., hydrocarbon group). In various embodiments, the “urea linkage” may contain 1, 2, 3, 4, 5, 6, 7, 8, 9, 10 or more than 10 urea groups.

The term “substituted,” when used to describe a chemical structure or moiety, refers to the chemical structure or moiety wherein one or more of its hydrogen atoms is substituted with a chemical moiety or functional group such as alcohol, alkoxy, alkanoyloxy, alkoxycarbonyl, alkenyl, alkyl (e.g., methyl, ethyl, propyl, t-butyl), alkynyl, alkylcarbonyloxy (—OC(O)alkyl), amide (—C(O)NH-alkyl- or -alkylNHC(O)alkyl), amine (such as alkylamino, arylamino, arylalkylamino), aryl, aryloxy, azo, carbamoyl (—NHC(O)O-alkyl- or —OC(O)NH-alkyl), carbamyl (e.g., CONH₂, as well as CONH-alkyl, CONH-aryl, and CONH-arylalkyl), carboxyl, carboxylic acid, cyano, ester, ether (e.g., methoxy, ethoxy), halo, haloalkyl (e.g., —CCl₃, —CF₃, —C(CF₃)₃), heteroalkyl, isocyanate, isothiocyanate, nitrile, nitro, phosphodiester, sulfide, sulfonamido (e.g., SO₂NH₂), sulfone, sulfonyl (including alkylsulfonyl, arylsulfonyl and arylalkylsulfonyl), sulfoxide, thiol (e.g., sulfhydryl, thioether) or urea (—NHCONH-alkyl-).

The term “micro” as used herein is to be interpreted broadly to include dimensions from about 1 micron to about 1000 microns.

The term “nano” as used herein is to be interpreted broadly to include dimensions less than about 1000 nm, less than about 500 nm, less than about 100 nm or less than about 50 nm.

The term “treatment”, “treat” and “therapy”, and synonyms thereof as used herein refer to both therapeutic treatment and prophylactic or preventative measures, wherein the object is to prevent or slow down (lessen) a medical condition, which includes but is not limited to diseases, symptoms and disorders. A medical condition also includes a body's response to a disease or disorder, e.g., inflammation. Those in need of such treatment include those already with a medical condition as well as those prone to getting the medical condition or those in whom a medical condition is to be prevented.

As used herein, the term “therapeutically effective amount” of a compound will be an amount of an active agent that is capable of preventing or at least slowing down (lessening) a medical condition, such as cancer, angiogenic eye disorders, ocular diseases in the anterior segment, ocular diseases in the posterior segment, neovascular related ophthalmic posterior segment diseases, retinal diseases, neovascular age-related macular degeneration (AMD) such as neovascular AMD, diabetic retinopathies, diabetic macular oedema (DMO), choroidal neovascularisation (CNV), central retinal vein occlusion (CRVO), corneal neovascularization, and retinal neovascularization. Dosages and administration of compounds, compositions and formulations of the present disclosure may be determined by one of ordinary skill in the art of clinical pharmacology or pharmacokinetics. See, for example, Mordenti and Rescigno, (1992) Pharmaceutical Research. 9:17-25; Morenti et al., (1991) Pharmaceutical Research. 8:1351-1359; and Mordenti and Chappell, “The use of interspecies scaling in toxicokinetics” in Toxicokinetics and New Drug Development, Yacobi et al. (eds) (Pergamon Press: NY, 1989), pp. 42-96. An effective amount of the active agent of the present disclosure to be employed therapeutically will depend, for example, upon the therapeutic objectives, the route of administration, and the condition of the patient. Accordingly, it may be necessary for the therapist to titer the dosage and modify the route of administration as required to obtain the optimal therapeutic effect.

The term “subject” as used herein includes patients and non-patients. The term “patient” refers to individuals suffering or are likely to suffer from a medical condition such as cancer, while “non-patients” refer to individuals not suffering and are likely to not suffer from the medical condition. “Non-patients” include healthy individuals, non-diseased individuals and/or an individual free from the medical condition. The term “subject” includes humans and animals. Animals include murine and the like. “Murine” refers to any mammal from the family Muridae, such as mouse, rat, and the like.

The terms “coupled” or “connected” as used in this description are intended to cover both directly connected or connected through one or more intermediate means, unless otherwise stated.

The term “associated with”, used herein when referring to two elements refers to a broad relationship between the two elements. The relationship includes, but is not limited to a physical, a chemical or a biological relationship. For example, when element A is associated with element B, elements A and B may be directly or indirectly attached to each other or element A may contain element B or vice versa.

The term “adjacent” used herein when referring to two elements refers to one element being in close proximity to another element and may be but is not limited to the elements contacting each other or may further include the elements being separated by one or more further elements disposed therebetween.

The term “and/or”, e.g., “X and/or Y” is understood to mean either “X and Y” or “X or Y” and should be taken to provide explicit support for both meanings or for either meaning.

Further, in the description herein, the word “substantially” whenever used is understood to include, but not restricted to, “entirely” or “completely” and the like. In addition, terms such as “comprising”, “comprise”, and the like whenever used, are intended to be non-restricting descriptive language in that they broadly include elements/components recited after such terms, in addition to other components not explicitly recited. For example, when “comprising” is used, reference to a “one” feature is also intended to be a reference to “at least one” of that feature. Terms such as “consisting”, “consist”, and the like, may in the appropriate context, be considered as a subset of terms such as “comprising”, “comprise”, and the like. Therefore, in embodiments disclosed herein using the terms such as “comprising”, “comprise”, and the like, it will be appreciated that these embodiments provide teaching for corresponding embodiments using terms such as “consisting”, “consist”, and the like. Further, terms such as “about”, “approximately” and the like whenever used, typically means a reasonable variation, for example a variation of +/−5% of the disclosed value, or a variance of 4% of the disclosed value, or a variance of 3% of the disclosed value, a variance of 2% of the disclosed value or a variance of 1% of the disclosed value.

Furthermore, in the description herein, certain values may be disclosed in a range. The values showing the end points of a range are intended to illustrate a preferred range. Whenever a range has been described, it is intended that the range covers and teaches all possible sub-ranges as well as individual numerical values within that range. That is, the end points of a range should not be interpreted as inflexible limitations. For example, a description of a range of 1% to 5% is intended to have specifically disclosed sub-ranges 1% to 2%, 1% to 3%, 1% to 4%, 2% to 3% etc., as well as individually, values within that range such as 1%, 2%, 3%, 4% and 5%. The intention of the above specific disclosure is applicable to any depth/breadth of a range.

Additionally, when describing some embodiments, the disclosure may have disclosed a method and/or process as a particular sequence of steps. However, unless otherwise required, it will be appreciated that the method or process should not be limited to the particular sequence of steps disclosed. Other sequences of steps may be possible. The particular order of the steps disclosed herein should not be construed as undue limitations. Unless otherwise required, a method and/or process disclosed herein should not be limited to the steps being carried out in the order written. The sequence of steps may be varied and still remain within the scope of the disclosure.

Furthermore, it will be appreciated that while the present disclosure provides embodiments having one or more of the features/characteristics discussed herein, one or more of these features/characteristics may also be disclaimed in other alternative embodiments and the present disclosure provides support for such disclaimers and these associated alternative embodiments.

### DESCRIPTION OF EMBODIMENTS

Exemplary, non-limiting embodiments of an anti-angiogenic agent, a method of preparing the anti-angiogenic agent, and related methods/uses are disclosed hereinafter.

In various embodiments, there is provided polymeric particles, more particularly, multi-block copolymer in the form of one or more micelles, wherein the copolymer comprises/consists essentially of/consists of a first poly(alkylene glycol) block, a second poly(alkylene glycol) block and a polyester block. Advantageously, in various embodiments, the multi-block copolymer micelles possess inherent/intrinsic anti-angiogenic properties. Accordingly, in various embodiments, as the micelles are anti-angiogenic on their own, the micelles may also be classified as an anti-angiogenic agent. Advantageously, embodiments of the micelles may directly reduce angiogenic cell proliferation, migration and tubing formation in the absence of other anti-angiogenic factors. Even more advantageously, embodiments of the polymeric micelles (e.g., nanomicelles) are capable of imparting surprisingly high anti-angiogenesis effects as compared to the corresponding hydrogel or free polymer forms. In fact, through the experiments conducted by the inventors, it has been shown that the anti-angiogenic effect is not achievable from the hydrogel form or the free polymer form.

Accordingly, in various embodiments, there is also provided an anti-angiogenic agent comprising the multi-block copolymer micelles.

In various embodiments, the multi-block copolymer is a tri-component multi-block polymer. For example, the multi-block polymer is made up of three different polymer blocks. Accordingly, in various embodiments, the first poly(alkylene glycol) block and the second poly(alkylene glycol) block are different from each other. In some embodiments, the multi-block copolymer comprises more than three polymeric blocks.

In various embodiments, the multi-block copolymer is a polymer that is not chemically cross-linked or is a non-cross-linking/non-cross-linked/non-crosslinkable polymer. The multi-block copolymer may form micelles via/due to physical interactions. Advantageously, in various embodiments, preparation/formation of the copolymer micelles disclosed herein does not require use of any additional chemical crosslinkers or crosslinking agents.

The multi-block copolymer may have at least one unit of the following structural sequence A-B-C, where A comprises a first poly(alkylene glycol), B comprises a second poly(alkylene glycol) and C comprises a polyester. In various embodiments, A is different from B and the positions of A, B and C may be interchanged among themselves. In various embodiments, the multi-block polymer may comprise a plurality of polymer blocks of the first poly(alkylene glycol), a plurality of polymer blocks of the second poly(alkylene glycol) and a plurality of polyester polymer blocks. In various embodiments, the multi-block copolymer comprises more than 3 polymeric blocks. The blocks may be randomly distributed/arranged within the copolymer.

In various embodiments, copolymer comprises at least one of urethane/carbamate, allophanate, carbonate, ester, urea linkages and/or combinations thereof. The first poly(alkylene glycol) polymer block, the second poly(alkylene glycol) polymer block and the polyester polymer block may be chemically coupled together by at least one of urethane/carbamate, allophanate, carbonate, ester, urea linkages or combinations thereof. In some embodiments, the first poly(alkylene glycol) polymer block, the second poly(alkylene glycol) polymer block and the polyester polymer block are chemically coupled together by at least a urethane/carbamate or allophanate linkage and optionally further coupled by one of carbonate, ester, urea linkages or combinations thereof. For example, each of the first poly(alkylene glycol) polymer block, the second poly(alkylene glycol) polymer block and the polyester polymer block may be linked to their respective adjacent block by at least one of urethane/carbamate, allophanate, carbonate, ester, urea linkages or combinations thereof. In various embodiments, the copolymer is a poly(ether ester) urethane polymer.

In various embodiments, the first and second poly(alkylene glycol) are independently selected from the group consisting of poly(ethylene glycol) (PEG), poly(propylene glycol) (PPG), poly(butylene glycol) and combinations thereof; and the polyester is selected from the group consisting of polycaprolactone (PCL) (e.g. poly(s-caprolactone), poly(lactic acid) (PLA), poly(lactic-co-glycolic acid) (PLGA), polyhydroxyalkanoate (PHA) (e.g., polyhydroxybutyrate (PHB), poly-3-hydroxybutyrate (P3HB), poly-4-hydroxybutyrate (P4HB), polyhydroxyvalerate (PHV), polyhydroxyhexanoate (PHH), polyhydroxyoctanoate (PHO)) and combinations thereof. In various embodiments, the first and second poly(alkylene glycol) are different. In one embodiment, the first poly(alkylene glycol) comprises PEG. In one embodiment, the second poly(alkylene glycol) comprises PPG. In one embodiment, the first poly(alkylene glycol) comprises PEG and the second poly(alkylene glycol) comprises PPG.

In various embodiments, the polyester is selected from the group consisting of polycaprolactone (PCL) (e.g. poly(s-caprolactone), poly(lactic acid) (PLA) and combinations thereof. In one embodiment, the polyester comprises PCL. In one embodiment, the first poly(alkylene glycol) comprises PEG, the second poly(alkylene glycol) comprises PPG and the polyester comprises PCL. In various embodiments, the multi-block copolymer is completely different from a poly(ethylene glycol)-poly(propylene glycol) (PEG-PPG), poly(propylene glycol)-poly(s-caprolactone) (PPG-PCL) or poly(ethylene glycol)-poly(s-caprolactone) (PEG-PCL) polymer.

In various embodiments, the molar ratio of the first poly(alkylene glycol) to the second poly(alkylene glycol) is in the range of from about 1:1 to about 10:1. The molar ratio of the first poly(alkylene glycol) to the second poly(alkylene glycol) may be about 1:1, about 2:1, about 3:1, about 4:1, about 5:1, about 6:1, about 7:1, about 8:1, about 9:1 or about 10:1. In various embodiments, the polyester is in an amount/concentration of from about 1 wt % to about 10 wt % of the multi-block copolymer. The polyester may be in an amount/concentration of about 1 wt %, about 2 wt %, about 3 wt %, about 4 wt %, about 5 wt %, about 6 wt %, about 7 wt %, about 8 wt %, about 9 wt %, or about 10 wt % of the multi-block copolymer. In some embodiments, the multi-block polymer contains about 1 wt % of poly(caprolactone). In various embodiments, the molar ratio of the first poly(alkylene glycol) block to the second poly(alkylene glycol) block to the polyester block in the copolymer is about 1 to 10:1:0.01 to 1.5.

In various embodiments, the copolymer is amphiphilic/amphipathic and comprises hydrophilic and hydrophobic parts.

In various embodiments, the anti-angiogenic agent comprises/consists essentially of/consists of the multi-block copolymer disclosed herein optionally with one or more bioactive(s) and water/aqueous medium/aqueous buffer (e.g. aqueous solution). Accordingly, the anti-angiogenic agent may exist as a composition or a formulation.

In various embodiments, as the copolymer exists as one or more micelles, it is not in the form of a hydrogel or free polymers. Advantageously, in various embodiments, the micelles are suitable for non-invasive intraocular penetration. It will be appreciated that when embodiments of the copolymer disclosed herein are dissolved in aqueous solvents, they are capable of undergoing three different forms of changes, depending on, for instance, the polymer concentration and temperature. For example, when the concentration is low (<critical micelle concentration (CMC)), the copolymer typically exists in the form of a free polymer solution and on the other hand when the concentration is increased to a concentration that is higher than CMC, micelles such as nanomicelles are typically formed. Finally, nanomicelles may be crosslinked together to form a hydrogel if the concentration is increased further. It will also be appreciated that although the polymer type may be the same, the three respective forms and their properties are significantly distinctive and different from each other. Without being bound by theory, it is believed that hydrogel forms are not able to spontaneously penetrate the cornea and migrate into the intraocular space. On the other hand, embodiments of the micelles are capable of spontaneously migrating (e.g. by diffusion) across one or more barriers (e.g. the one or more barriers separating the intraocular space and the external environment such as the sclera and/or cornea) into intraocular space (e.g, posterior segment of the eye, retina, vitreous cavity, vitreous humor etc.).

In various embodiments, the copolymer micelle is self-assembled/formed/generated in the presence of water, buffer or other aqueous medium (e.g., aqueous solution). In various embodiments, the copolymer micelle is self-assembled/formed/generated at a concentration that is higher than the critical micelle concentration (CMC) but lower than the sol-gel transition concentration of the copolymer. In an example, the polymeric/copolymer micelle comprises poly(ethylene glycol), poly(propylene glycol), poly(s-caprolactone) (herein termed “EPC”) is self-assembled into micelles (e.g. nanomicelles) at the applicable concentration in the presence of an anti-VEGF in an aqueous solution. Advantageously, the inventors have surprising shown that EPC polymeric micelles complexed with anti-VEGF drugs are able to show ocular barrier penetration activity by penetrating the cornea and reaching the retina. For example, the inventors have found that both corneal- and scleral-penetration was enhanced by complexing an anti-VEGF such as aflibercept with EPCs (e.g. nano-EPCs or nEPCs), which result in 4 times increased aflibercept detection within mice vitreous after a single topical drop as compared to aflibercept alone. Without being bound by theory, it is believed that the enhanced penetration enhancement relates to the enhanced intracellular uptake when anti-VEGFs are complexed with EPC micelles.

In various embodiments, the copolymer has a critical micelle concentration (CMC) of from about 0.01 wt % to about 2.00 wt %, from about 0.05 wt % to about 1.95 wt %, from about 0.10 wt % to about 1.90 wt %, from about 0.15 wt % to about 1.85 wt %, from about 0.20 wt % to about 1.80 wt %, from about 0.25 wt % to about 1.75 wt %, from about 0.30 wt % to about 1.70 wt %, from about 0.35 wt % to about 1.65 wt %, from about 0.40 wt % to about 1.60 wt %, from about 0.45 wt % to about 1.55 wt %, from about 0.50 wt % to about 1.50 wt %, from about 0.55 wt % to about 1.45 wt %, from about 0.60 wt % to about 1.40 wt %, from about 0.65 wt % to about 1.35 wt %, from about 0.70 wt % to about 1.30 wt %, from about 0.75 wt % to about 1.25 wt %, from about 0.80 wt % to about 1.20 wt %, from about 0.85 wt % to about 1.15 wt %, from about 0.90 wt % to about 1.10 wt %, from about 0.95 wt % to about 1.05 wt %, or about 1.00 wt % at a temperature of from about 20° C. to about 40° C. In various embodiments, the CMC is about 0.105 wt % when measured at 25° C. and about 0.046 wt % when measured at 37° C. The CMC may be higher than about 0.105 wt % when the temperature is reduced to 20° C.

In various embodiments, the copolymer is present in the composition or the formulation at a total polymer concentration in the range of from about 0.01 wt % to about 6.00 wt %, from about 0.02 wt % to about 5.50 wt %, from about 0.03 wt % to about 5.00 wt %, from about 0.04 wt % to about 4.50 wt %, from about 0.05 wt % to about 4.00 wt %, from about 0.06 wt % to about 3.50 wt %, from about 0.07 wt % to about 3.00 wt %, from about 0.08 wt % to about 2.50 wt %, from about 0.09 wt % to about 2.00 wt %, from about 0.10 wt % to about 1.90 wt %, from about 0.15 wt % to about 1.85 wt %, from about 0.20 wt % to about 1.80 wt %, from about 0.25 wt % to about 1.75 wt %, from about 0.30 wt % to about 1.70 wt %, from about 0.35 wt % to about 1.65 wt %, from about 0.40 wt % to about 1.60 wt %, from about 0.45 wt % to about 1.55 wt %, from about 0.50 wt % to about 1.50 wt %, from about 0.55 wt % to about 1.45 wt %, from about 0.60 wt % to about 1.40 wt %, from about 0.65 wt % to about 1.35 wt %, from about 0.70 wt % to about 1.30 wt %, from about 0.75 wt % to about 1.25 wt %, from about 0.80 wt % to about 1.20 wt %, from about 0.85 wt % to about 1.15 wt %, from about 0.90 wt % to about 1.10 wt %, from about 0.95 wt % to about 1.05 wt %, or about 1.00 wt %. In various embodiments, the total polymer concentration is lower than the sol-gel transition concentration or the concentration at which the polymer forms/converts into a gel form. For example, the sol-gel transition concentration may be from about 2.0 wt % to about 10.0 wt %, and therefore the total polymer concentration may be no more than about 10.0 wt %, no more than about 9.0 wt %, no more than about 8.0 wt %, no more than about 7.0 wt %, no more than about 6.0 wt %, no more than about 5.0 wt %, no more than about 4.0 wt %, no more than about 3.0 wt % or no more than about 2.0 wt %.

In various embodiments, the composition or formulation comprises an aqueous medium or aqueous buffer. The aqueous medium may be a balanced salt solution. In various embodiments, the balanced salt solution is a solution having a physiological pH and isotonic salt concentration. In various embodiments, the balanced salt solution comprises at least one of sodium, potassium, calcium and magnesium salts such as calcium chloride, potassium chloride, magnesium chloride, sodium acetate, sodium citrate and sodium chloride.

In various embodiments, the anti-angiogenic agent has a high water content of more than about 60%, about 65%, about 70%, about 75%, about 80%, about 85%, about 90%, about 95%, about 96%, about 97%, about 98%, about 99% %, about 99.5% or about 99.9% by weight.

In various embodiments, the micelle has a hydrodynamic size of from about 1.0 nm to about 100.0 nm, from about 2.0 nm to about 99.0 nm, from about 5.0 nm to about 95.0 nm, from about 10.0 nm to about 90.0 nm, from about 15.0 nm to about 85.0 nm, from about 20.0 nm to about 80.0 nm, from about 25.0 nm to about 75.0 nm, from about 30.0 nm to about 70.0 nm, from about 35.0 nm to about 65.0 nm, from about 40.0 nm to about 60.0 nm, from about 45.0 nm to about 55.0 nm, or about 50.0. Accordingly, in various embodiments, the micelle is a nanomicelle, i.e. micelle in the nano-sized. In various embodiments where the micelles are nano-sized, the material property may be significantly different from the bulk material in the way it interacts with cells. Thus, the advantageous properties of the nanomicelles (e.g. anti-angiogenesis effect and ability to penetrate the structural anatomy of the eye such as the cornea to reach the intraocular space) are unexpected and cannot be simply gleaned from the bulk material properties of the copolymer (e.g. in hydrogel form or free form).

In various embodiments, the copolymer micelle comprises a hydrophobic core. Advantageously, hydrophobic drugs or bioactives may be loaded within the hydrophobic core. Examples of hydrophobic drugs include but are not limited to paclitaxel, doxorubicin, teniposide, etoposide, daunomycin, methotrexate, mitomycin C, indomethacin, ibuprofen, cyclosporine, and biphenyl dimethyl dicarboxylate (DDB).

In various embodiments, the copolymer micelle further comprises one or more bioactive(s) that is/are complexed with, encapsulated by or incorporated into the copolymer/micelle. Accordingly, in various embodiments, the copolymer micelle may exist as a complex. Advantageously, various embodiments of the copolymer micelle may serve as a drug delivery system or a drug carrier/nanocarrier. For example, a drug delivery system comprising an EPC polymeric micelle that forms a complex with an anti-VEGF drug may be used for topical application to the eye and still reach the retina.

In various embodiments, the copolymer micelle has an encapsulation efficiency/loading capacity of at least about 1%, at least about 5%, at least about 10%, at least about 15%, at least about 20%, at least about 25%, at least about 30%, at least about 35%, at least about 40%, at least about 45%, at least about 50%, at least about 55% or at least about 60%. In various embodiments, the loading capacity of the bioactive increases in a micellar concentration-dependent manner.

In various embodiments, the bioactive comprises small molecules, large macromolecules (e.g., molecular weight of more than 50 kDa), biological macromolecules (e.g., carbohydrates, lipids, proteins, and nucleic acids), therapeutics and/or drug molecules (e.g., anti-tumour drugs) that are capable of providing a biological effect, therapeutic effect, prophylactic effect or combinations thereof. In various embodiments, the bioactive comprises macromolecular anti-VEGF drugs/agents.

In various embodiments, the copolymer micelle having an intrinsic anti-angiogenic property is capable of working synergistically with the bioactive(s) to provide a combined and enhanced therapeutic effect. For example, the bioactive may be one that have an anti-angiogenesis effect. Advantageously, this enhances or synergises with the anti-angiogenesis effect that is already intrinsically or inherently present in embodiments of the copolymer micelles disclosed herein. For example, in embodiments where the bioactive(s) comprise anti-VEGFs, the intrinsic anti-angiogenic property of the copolymer micelle together with the anti-angiogenic property of anti-VEGFs work synergistically to provide an overall enhanced anti-angiogenic effect. Advantageously, the copolymer micelle may be in the form of a topical micelle or nanomicelle having an enhanced anti-vascular endothelial growth factor (anti-VEGF) penetration and intrinsic antiangiogenic effects for synergistic treatment of diseases responsive to an anti-angiogenesis effect, e.g. such as neovascular retinal diseases. In various embodiments, the polymeric/copolymer micelle comprises poly(ethylene glycol), poly(propylene glycol), poly(s-caprolactone) (i.e. EPC) forms an EPC-drug complex with an anti-VEGF drug to treat retinal neovascular diseases, for example, as a topical eye drop (e.g. in an aqueous solution).

Thus, in various embodiments, the bioactive comprises an anti-vascular endothelial growth factor (anti-VEGF). Examples of anti-VEGF include but are not limited to bevacizumab, aflibercept, ranibizumab, brolucizumab or the like.

In various embodiments, the bioactive comprises a tyrosine kinase inhibitors (TKI). Examples of TKI include but are not limited to brivanib, cediranib, dovitinib, sunitinib, sorafenib, vatalanib or the like.

In various embodiments, the bioactive comprises an anti-cancer drug. Examples of anti-cancer drug include but are not limited to docetaxel, mitoxantrone, gemcitabine, capecitabine, oxaliplatin, interferon, sunitinib, sorafenib, carboplatinum, doxorubicin, methotrexate, vincristine, vinorelbine, pemetrexed, gefitinib, etoposide, irinotecan, cyclophosphamide, topotecan, cyclophosphamide, paclitaxel, mitomycin, bevacizumab, trastuzumab, cetuximab, temozolomide, procarbazine, or the like.

While in some embodiments, the copolymer micelles may be able to act as a carrier for genes, small molecular weight drugs such as TKIs (e.g. sunitinib), anticancer drugs (e.g. avastin) etc, it will be appreciated that in various embodiments, the bioactive used excludes genes, small molecular weight drugs such as TKIs (e.g. sunitinib), anticancer drugs (e.g. avastin) etc. For example, in various embodiments, the anti-VEGFs used as the bioactives are completely different from the above in terms of structure and function.

Furthermore, it will be appreciated that although the mode of delivery may be topical for various embodiments disclosed herein, the mechanism of delivery may still be different from those topical applications used in the art. For instance, embodiments of the present disclosure are different from those of the art that topically delivers avastin to the posterior segment of eye using cell penetration peptides (CPP)-facilitated penetration or annexin A5-associated liposomes. It will be appreciated that in such a method, the penetration enhancement comes from the peptide such as Annexin A5, which is a protein and not the copolymer micelle or nanomicelle disclosed herein. Embodiments of the copolymer micelles disclosed herein are not peptides nor are they protein-based carriers. It will be appreciated that cell penetration peptides are protein based, and there are limitations in real human applications. Furthermore, embodiments of the copolymer micelles disclosed herein are also not liposomes.

In various embodiments, the copolymer micelle enhances/increases the intraocular penetration of bioactive(s) across barrier layers such as the sclera and/or cornea layers and enhances/increases intracellular uptake of the bioactive(s).

The bioactive may be present at a concentration of from about 0.1 mg/mL to about 100.0 mg/mL. In various embodiments, the bioactive is present at a concentration of about 0.1 mg/mL, about 0.2 mg/mL, about 0.5 mg/mL, about 1.0 mg/mL, about 2.0 mg/mL, about 5.0 mg/mL, about 10.0 mg/mL, about 15.0 mg/mL, about 20.0 mg/mL, about 25.0 mg/mL, about 30.0 mg/mL, about 35.0 mg/mL, about 40.0 mg/mL, about 45.0 mg/mL, about 50.0 mg/mL, about 55.0 mg/mL, about 60.0 mg/mL, about 65.0 mg/mL, about 70.0 mg/mL, about 75.0 mg/mL, about 80.0 mg/mL, about 85.0 mg/mL, about 90.0 mg/mL, about 95.0 mg/mL, about 98.0 mg/ml, about 99.0 mg/mL or about 100.0 mg/mL.

In various embodiments, the copolymer micelle is biocompatible and/or non-toxic and/or does not elicit an inflammatory or adverse immune response in the body of an animal or human, particularly in the eye of an animal or human (e.g., corneal epithelial cells or the corneal barrier).

In various embodiments, the copolymer is substantially devoid of heavy metals and/or contaminants. For example, the copolymer may be substantially devoid of antimony and/or arsenic and/or cadmium and/or cobalt and/or copper and/or lead and/or lithium and/or mercury and/or nickel and/or vanadium.

In various embodiments, the copolymer is substantially devoid of solvent contaminants. For example, the copolymer may be substantially devoid of benzene and/or carbon tetrachloride and/or 1,2-dichloroethane and/or 1,1-dichloroethene and/or 1,1,1-trichloroethane and/or acetonitrile and/or chlorobenzene and/or chloroform and/or cyclohexane and/or 1,2-dichloroethene and/or dichloromethane and/or 1,2-dimethoxyethane and/or N,N-dimethylacetamide and/or N,N-dimethylformamide and/or 1,4-dioxane and/or 2-ethoxyethanol and/or ethyleneglycol and/or formamide and/or hexane and/or methanol and/or 2-methoxyethanol and/or methylbutylketone and/or methylcyclohexane and/or N-methylpyrrolidone and/or nitromethane and/or pyridine and/or sulfolane and/or tetrahydrofuran and/or tetralin and/or toluene and/or 1,1,2-trichloroethene and/or xylene (m-, p-, o-isomers) and/or acetic acid and/or acetone and/or anisole and/or 1-butanol and/or 2-butanol and/or butyl acetate and/or tert-butylmethyl ether and/or cumene and/or dimethyl sulfoxide and/or ethanol and/or ethyl acetate and/or ethyl ether and/or ethyl formate and/or formic acid and/or heptane and/or isobutyl acetate and/or isopropyl acetate and/or methyl acetate and/or 3-methyl-1-butanol and/or methylethylketone and/or methylisobutylketone and/or 2-methyl-1-propanol and/or pentane and/or 1-pentanol and/or 1-propanol and/or 2-propanol and/or propyl acetate.

In various embodiments, the copolymer has a short residence time and/or is capable of being degraded naturally in the animal body within about 6 months, within about 5 months, within about 4 months, within about 3 months, or within about 2 months. In some embodiments, the copolymer has a short residence time and/or is capable of being degraded naturally in the animal body in the time period of between about 2 months and about 6 months.

In various embodiments, the copolymer is biocompatible and/or non-toxic and/or does not elicit an inflammatory or adverse immune response in the body of an animal or human, particularly in the eye of an animal or human.

In various embodiments, the copolymer or at least one or more of the blocks within the copolymer is/are biodegradable and/or can be broken down naturally. In some examples, all the polymeric blocks are biodegradable.

Advantageously, in various embodiments, the copolymer micelle is capable of being administered/delivered to an animal or human repeatedly/consecutively/consistently over a period of at least about 1 day, about 2 days, about 3 days, about 4 days, about 5 days, about 6 days, about 7 days, about 14 days or about 21 days without causing change to the morphology, organisation, structure and/or function of the eye (e.g., refractive components such as cornea, lens, corneal epithelial and endothelial cells). For example, repeated administration/delivery does not result in accelerated cataract formation. The route of administration or delivery may be topical and/or non-invasive. Accordingly, in various embodiments, anti-angiogenic agent is formulated as a topical ophthalmic formulation such as in the form of an eye drop.

In various embodiments, there is also provided a method of preparing the anti-angiogenic agent disclosed herein, the method comprising adding a copolymer to an aqueous medium at a concentration that is no less than the critical micelle concentration of the copolymer but no more than the sol-gel transition concentration of the copolymer, to form micelles, wherein the copolymer comprises a first poly(alkylene glycol) block, a second poly(alkylene glycol) block and a polyester block. The copolymer may be in a powder form or dry form prior to adding to the aqueous medium. It will be appreciated that the anti-angiogenic agent, the copolymer, the aqueous medium, the micelle, the first poly(alkylene glycol) block, the second poly(alkylene glycol) block and the polyester block etc may possess one or more properties or characteristics as discussed earlier.

In various embodiments, the copolymer is added to the aqueous medium such that a total polymer concentration in the range of from about 0.01 wt % to about 6.00 wt %, from about 0.02 wt % to about 5.50 wt %, from about 0.03 wt % to about 5.00 wt %, from about 0.04 wt % to about 4.50 wt %, from about 0.05 wt % to about 4.00 wt %, from about 0.06 wt % to about 3.50 wt %, from about 0.07 wt % to about 3.00 wt %, from about 0.08 wt % to about 2.50 wt %, from about 0.09 wt % to about 2.00 wt %, from about 0.10 wt % to about 1.90 wt %, from about 0.15 wt % to about 1.85 wt %, from about 0.20 wt % to about 1.80 wt %, from about 0.25 wt % to about 1.75 wt %, from about 0.30 wt % to about 1.70 wt %, from about 0.35 wt % to about 1.65 wt %, from about 0.40 wt % to about 1.60 wt %, from about 0.45 wt % to about 1.55 wt %, from about 0.50 wt % to about 1.50 wt %, from about 0.55 wt % to about 1.45 wt %, from about 0.60 wt % to about 1.40 wt %, from about 0.65 wt % to about 1.35 wt %, from about 0.70 wt % to about 1.30 wt %, from about 0.75 wt % to about 1.25 wt %, from about 0.80 wt % to about 1.20 wt %, from about 0.85 wt % to about 1.15 wt %, from about 0.90 wt % to about 1.10 wt %, from about 0.95 wt % to about 1.05 wt %, or about 1.00 wt %. In various embodiments, the total polymer concentration is lower than the sol-gel transition concentration or the concentration at which the polymer forms/converts into a gel form. For example, the sol-gel transition concentration may be from about 2.0 wt % to about 10.0 wt %, and therefore the total polymer concentration may be no more than about 10.0 wt %, no more than about 9.0 wt %, no more than about 8.0 wt %, no more than about 7.0 wt %, no more than about 6.0 wt %, no more than about 5.0 wt %, no more than about 4.0 wt %, no more than about 3.0 wt % or no more than about 2.0 wt %.

In various embodiments, the method further comprising coupling the first poly(alkylene glycol) block, the second poly(alkylene glycol) block and the polyester block together by at least one of urethane/carbamate, allophanate, carbonate, ester, urea linkages and/or combinations thereof. For example, each of the first poly(alkylene glycol) polymer block, the second poly(alkylene glycol) polymer block and the polyester polymer block may be linked to their respective adjacent block by at least one of urethane/carbamate, allophanate, carbonate, ester, urea linkages or combinations thereof.

In some embodiments, the first poly(alkylene glycol) polymer block, the second poly(alkylene glycol) polymer block and the polyester polymer block are chemically coupled together by at least a urethane/carbamate or allophanate linkage and optionally further coupled by one of carbonate, ester, urea linkages or combinations thereof. In various embodiments, the copolymer is a poly(ether ester) urethane polymer.

In various embodiments of the method, the first and second poly(alkylene glycol) are independently selected from the group consisting of poly(ethylene glycol) (PEG), poly(propylene glycol) (PPG), poly(butylene glycol) and combinations thereof; and the polyester is selected from the group consisting of polycaprolactone (PCL) (e.g. poly(s-caprolactone), poly(lactic acid) (PLA), poly(lactic-co-glycolic acid) (PLGA), polyhydroxyalkanoate (PHA) (e.g., polyhydroxybutyrate (PHB), poly-3-hydroxybutyrate (P3HB), poly-4-hydroxybutyrate (P4HB), polyhydroxyvalerate (PHV), polyhydroxyhexanoate (PHH), polyhydroxyoctanoate (PHO)) and combinations thereof. In various embodiments, the first and second poly(alkylene glycol) are different. In one embodiment, the first poly(alkylene glycol) comprises PEG. In one embodiment, the second poly(alkylene glycol) comprises PPG. In one embodiment, the first poly(alkylene glycol) comprises PEG and the second poly(alkylene glycol) comprises PPG.

In various embodiments of the method, the polyester is selected from the group consisting of polycaprolactone (PCL) (e.g. poly(s-caprolactone), poly(lactic acid) (PLA) and combinations thereof. In one embodiment, the polyester comprises PCL.

In various embodiments, the method comprises coupling or mixing PEG, PPG and PCL together.

In various embodiments of the method, the first poly(alkylene glycol) and the second poly(alkylene glycol) are coupled or mixed in a molar ratio falling in the range of from about 1:1 to about 10:1. The molar ratio of the first poly(alkylene glycol) to the second poly(alkylene glycol) coupled or mixed together may be about 1:1, about 2:1, about 3:1, about 4:1, about 5:1, about 6:1, about 7:1, about 8:1, about 9:1 or about 10:1. In various embodiments, the polyester is coupled or mixed in an amount/concentration of from about 1 wt % to about 10 wt % of the multi-block copolymer. The polyester may be coupled or mixed in an amount/concentration of about 1 wt %, about 2 wt %, about 3 wt %, about 4 wt %, about 5 wt %, about 6 wt %, about 7 wt %, about 8 wt %, about 9 wt %, or about 10 wt % of the multi-block copolymer. In some embodiments, the multi-block polymer contains about 1 wt % of poly(caprolactone) after the coupling step. In various embodiments, the first poly(alkylene glycol) block, the second poly(alkylene glycol) block, and the polyester block in the copolymer are coupled in a molar ratio of from about 1 to 10:1:0.01 to 1.5.

In various embodiments, the coupling and/or mixing step is performed at an elevated temperature of from about 70° C. to about 150° C., from about 72° C. to about 148° C., from about 74° C. to about 146° C., from about 76° C. to about 144° C., from about 78° C. to about 142° C., from about 80° C. to about 140° C., from about 82° C. to about 138° C., from about 84° C. to about 136° C., from about 86° C. to about 134° C., from about 88° C. to about 132° C., from about 90° C. to about 130° C., from about 92° C. to about 128° C., from about 94° C. to about 126° C., or from about 96° C. to about 124° C., from about 98° C. to about 122° C., from about 100° C. to about 120° C., from about 102° C. to about 118° C., from about 104° C. to about 116° C., from about 106° C. to about 114° C., from about 108° C. to about 112° C., or about 110° C.

In various embodiments, the coupling and/or mixing step is carried out for at least about 1 hour, at least about 2 hours, at least about 3 hours, at least about 4 hours, at least about 5 hours, at least about 6 hours, at least about 7 hours, at least about 8 hours, at least about 9 hours, at least about 10 hours, at least about 11 hours, at least about 12 hours, at least about 14 hours, at least about 16 hours, at least about 18 hours, at least about 20 hours, at least about 22 hours, at least about 24 hours, at least about 26 hours, at least about 28 hours, at least about 30 hours, at least about 32 hours, at least about 34 hours, at least about 36 hours, at least about 38 hours, at least about 40 hours, at least about 42 hours, at least about 44 hours, at least about 46 hours, or at least about 48 hours.

In various embodiments, the coupling and/or mixing step is performed in the absence of air and/or water/moisture and/or in the presence of an inert gas such as nitrogen.

In various embodiments, the coupling step is carried out in the presence of a coupling agent. In various embodiments, the coupling agent comprises an isocyanate monomer that contains at least two (two or more) isocyanate functional groups. The coupling agent may be a diisocyanate selected from the group consisting of hexamethylene diisocyanate, tetramethylene diisocyanate, cyclohexane diisocyanate, tetramethylxylene diisocyanate, dodecylene diisocyanate, tolylene 2,4-diisocyanate and tolylene 2,6-diisocyanate. In various embodiments, a linear polymer is formed by using an isocyanate monomer that contains two isocyanate functional groups as the coupling agent. In various embodiments, the coupling agent contains no more than 2 isocyanates, which may otherwise form a branched polymer that may not be capable of imparting the same cell penetration effect as a linear polymer.

In various embodiments, the coupling step is carried out in the presence of a solvent. The solvent comprises an anhydrous solvent selected from the group consisting of toluene, benzene, xylene, halogenated organic solvents, halogenated alkane solvents, chlorinated solvents, dichloromethane, dichloroethane, tetrachloromethane and chloroform (or trichloromethane).

In various embodiments, the coupling step is carried out in the presence of a catalyst. The catalyst may be a metal catalyst. In various embodiments, the metal catalyst comprises a tin catalyst selected from the group consisting of alkyltin compounds, aryltin compounds and dialkyltin diesters such as dibutyltin dilaurate, dibutyltin diacetate, dibutyltin dioctanoate and dibutyltin distearate.

In various embodiments, the method further comprising complexing or encapsulating one or more bioactive(s) with the micelle. In various embodiments, the bioactive may be already present in the aqueous medium (e.g., aqueous solution, water, buffer etc) prior to the step of adding the copolymer to the aqueous medium. Thus, in various embodiments, the bioactive(s) may be added to the aqueous solutions prior to the formation of the copolymer micelles. In various embodiments, the encapsulation of the bioactive(s) in the micelle may take place during the formation of the micelle itself (e.g. self-formation of micelle during addition of copolymer to the aqueous medium). The one or more bioactive(s) may possess one or more properties or characteristics as discussed earlier. For example, the bioactive may be an anti-vascular endothelial growth factor (anti-VEGF) and/or may be selected the group consisting of bevacizumab, aflibercept, ranibizumab and brolucizumab.

In various embodiments, by directly mixing the copolymer micelles (e.g. nEPCs) with anti-VEGFs, copolymer micelles and anti-VEGFs complexes are formed. Increasing the copolymer (e.g. EPC) concentration increases entrapment efficiency of anti-VEGFs, therefore, enhancing the barrier penetration efficiency of anti-VEGFs. In one example, when EPC micelles & anti-VEGF (e.g., Aflibercept) complexes (herein termed “nEPCs+A”) are formed, it was found that one or more of the following properties may be enhanced: (i) corneal permeability of anti-VEGF (in mice), (ii) amount of anti-VEGF in vitreous humour after a single topical administration, (iii) ex vivo porcine sclera and corneal permeability, (iv) in vitro intracellular uptake, (iv) anti-angiogenic effect due to the nEPCs working synergistically. In various embodiments therefore, there is also provided a method of preparing an EPC anti-VEGF drug complex, the method comprising mixing the EPC polymeric micelle with an anti-VEGF drug.

In various embodiments, the bioactive is added to, complexed with or encapsulated by the copolymer micelle(s) at a concentration of from about 0.1 mg/mL to about 100.0 mg/mL. In various embodiments, the bioactive is present at a concentration of about 0.1 mg/mL, about 0.2 mg/mL, about 0.5 mg/mL, about 1.0 mg/mL, about 2.0 mg/mL, about 5.0 mg/mL, about 10.0 mg/mL, about 15.0 mg/mL, about 20.0 mg/mL, about 25.0 mg/mL, about 30.0 mg/mL, about 35.0 mg/mL, about 40.0 mg/mL, about 45.0 mg/mL, about 50.0 mg/mL, about 55.0 mg/mL, about 60.0 mg/mL, about 65.0 mg/mL, about 70.0 mg/mL, about 75.0 mg/mL, about 80.0 mg/mL, about 85.0 mg/mL, about 90.0 mg/mL, about 95.0 mg/mL, about 98.0 mg/ml, about 99.0 mg/mL or about 100.0 mg/mL.

In various embodiments, the method further comprises removing the multi-block copolymer of/from contaminants; and solubilizing the multi-block copolymer in aqueous medium to form a multi-block micelle. The step of removing the multi-block polymer of/from contaminants may comprise purifying and/or washing the multi-block copolymer. The step of solubilizing the multi-block copolymer in aqueous medium may comprise redissolving the polymer (e.g. final polymer powder) in a balanced salt solution (BSS). In various embodiments, BSS is water-based.

In various embodiments, the step of removing the multi-block copolymer of/from contaminants comprises dialysis to remove unreacted reactants, solvents and catalyst (e.g. extensive dialysis to remove unreacted PEG, solvents and metallic catalyst etc).

It will be appreciated that although it may be carried out, purifying and/or washing the copolymer micelles is optional in various embodiments. In various embodiments, the method of preparing the copolymer micelles disclosed herein is essentially a simple mixing process and can be applied without further purification e.g. no post-encapsulation purification or washing may be required. For example, EPC forms nanomicelles (nEPCs) in aqueous solution and complexes with aflibercept by a simple mixing process and can be applied without further purification or washing. Advantageously, the direct mixing complexation method minimizes the denaturation of protein-drug induced by mechanical stirring, heating or organic solvents. Moreover, embodiments of this straightforward complex-preparation process simplify the manufacturing process and make on-site drug encapsulation possible. Even more advantageously by removing the need for purification and/or washing, a significant amount of drug loss can be prevented, thus allowing the drug loading amount to be maximized.

The method may also further comprise sterilizing the materials used in the preparation of the anti-angiogenic agent, for example by autoclaving methods or techniques.

In various embodiments, there is also provided a method of preparing the copolymer micelle comprising (i) coupling one or more polymer blocks selected from the group consisting of poly(alkylene glycol), polyester and combinations thereof to form a copolymer, optionally the polymer blocks are chemically coupled/linked by at least urethane/carbamate linkage(s); and (ii) solubilizing the copolymer in water, buffer or other aqueous medium (e.g., aqueous solution) to form the copolymer micelle. The coupling step may comprise mixing one or more polymers selected from the group consisting of poly(alkylene glycol), polyester and combinations thereof with a coupling agent in the presence of a catalyst and a suitable solvent to form the copolymer. The method may further comprise (iii) mixing one or more bioactive(s) with the copolymer micelle. In various embodiments, the bioactive may be already present in the water, buffer or other aqueous medium (e.g., aqueous solution) of step (ii) prior to solubilizing the copolymer.

In various embodiments, there is also provided an anti-angiogenic agent as disclosed herein for the prophylaxis or treatment of an eye disorder, the use of the anti-angiogenic agent disclosed herein in the manufacture of a medicament for the prophylaxis or treatment of an eye disorder and/or a method of preventing or treating an eye disorder, comprising administering (e.g. in a therapeutically effective amount) the anti-angiogenic agent disclosed herein to a subject in need thereof. The eye disorder may be selected from the group consisting of angiogenic eye disorders, ocular diseases in the anterior segment, ocular diseases in the posterior segment, neovascular related ophthalmic posterior segment diseases, retinal diseases, neovascular age-related macular degeneration (AMD) such as neovascular AMD, diabetic retinopathies, diabetic macular oedema (DMO), choroidal neovascularisation (CNV), central retinal vein occlusion (CRVO), corneal neovascularization, and retinal neovascularization.

In various embodiments, the anti-angiogenic agent is applied or administered topically to the subject (e.g. the eye of the subject) in need thereof. Advantageously, this is different from those of art which involve the use of intravitreal injection (IVT). For instance, it will be appreciated that the mainline for treating age-related macular degeneration (AMD) is intravitreal injections. On the contrary, embodiments of the present disclosure comprise a less invasive topical application, e.g., in the form of an eye drop as embodiments of the copolymer micelles can cross through ocular barriers after topical eye drop application to deliver to the retinal region of the eye. Indeed, this represents a significant contribution to the art.

In various embodiments, there is also provided an ocular delivery system for delivery of an anti-vascular endothelial growth factor (anti-VEGF) in the eye, the system comprising the copolymer micelle disclosed herein; and the anti-VEGF encapsulated by the copolymer micelle. In various embodiments, the ocular delivery system is a non-invasive ocular delivery system.

In various embodiments, there is also provided an anti-angiogenesis system for delivery of an anti-vascular endothelial growth factor (anti-VEGF), the system comprising the copolymer micelle disclosed herein; and the anti-VEGF encapsulated by the copolymer micelle.

In various embodiments, there is also provided an anti-angiogenic agent as disclosed herein for the prophylaxis or treatment of a proliferative disease such as cancer, the use of the anti-angiogenic agent disclosed herein in the manufacture of a medicament for the prophylaxis or treatment of a proliferative disease such as cancer or a method of preventing and/or treating a proliferative disease such as cancer, comprising administering (e.g. in a therapeutically effective amount) the anti-angiogenic agent disclosed herein to a subject in need thereof. Advantageously, as embodiments of the anti-angiogenic agent or copolymer micelles exhibit anti-angiogenic effects, they may be used as agents or carriers to block or reduce angiogenesis in undesirable proliferative cells, for example tumour or cancer cells that have uncontrolled/undesired proliferation, thereby blocking nutrients and oxygen supply to these cells and effectively “starving” them.

In various embodiments, there is also provided an anti-angiogenic agent, a copolymeric particle/micelle, a micelle drug delivery system, a drug carrier, an ocular delivery system, an anti-angiogenesis system disclosed herein for use in medicine (e.g. for the treatment in one or more of the diseases or conditions mentioned herein) and/or for use in drug delivery (e.g., in the eye).

### BRIEF DESCRIPTION OF FIGURES

FIG. 1 is a schematic diagram of a multi-block copolymer (e.g., EPC polymer 100) in accordance with various embodiments disclosed herein. As shown in FIG. 1, the EPC polymer 100 can be self-assembled into micelles (e.g., polymeric nanomicelles (nEPCs) 102) in a buffer 104. nEPCs 102 alone are able to inhibit angiogenesis in-vitro and ex-vivo. As shown in FIG. 1, aflibercept 108 can be encapsulated by nEPCs through direct mixing to form nEPC+aflibercept (nEPCs+A) complexes 106. When administered topically on the murine cornea, nEPCs functioned as a drug carrier to deliver aflibercept across the cornea to achieve therapeutic concentrations in the retina of laser-induced disease models of choroidal neovascularisation (CNV).

FIG. 2 to FIG. 4 shows characterization of EPC nanomicelle (nEPCs) and its interaction with aflibercept in accordance with various embodiments disclosed herein.

FIG. 2 shows critical micelle concentration (CMC) values of EPC determined using a dye solubilisation method where changes in absorbance of hydrophobic dye 1,6-diphenyl-1,3,5-hexatriene (DPH) was monitored in accordance with various embodiments disclosed herein. The CMC for nanomicelle formation was found to be 0.046 wt % at 37° C.

FIG. 3 shows interactions of nEPC with Aflibercept studied using fluorescence emitted by Rhodamine-labelled Aflibercept (Rho-A) and observing the fluorescence intensity changes when the same amount of Rho-A was added to different concentrations of EPC in accordance with various embodiments disclosed herein. Bottom figure shows graph presented in terms of EPC concentration (wt %) while top figure shows graph presented in terms of log EPC concentration. At 0.05 wt % of EPC, free Rho-A was abundant, suggested by the high fluorescence intensity measured. However, there was subsequently a sharp reduction of fluorescence intensity up to around 0.5 wt % (labelled as “2” in the bottom figure), suggesting the incorporation of Rho-A into the micelles when nEPC was formed. In the bottom figure, “1” indicates 0.05 wt % and “2” indicates 0.2 wt %.

FIG. 4 shows the interaction between the micelle polymeric components and Aflibercept analysed using ¹H NMR, showing differences in the resonances of EPC peaks for PEG at 3.57 ppm (labelled a) and PPG at 1.03 ppm (labelled b), with and without Aflibercept (spectra are referenced to residual solvent peak of water at 4.66 ppm) in accordance with various embodiments disclosed herein.

FIG. 5 shows the absorbance spectra of 1,6-diphenyl-1,3,5-hexatriene (DPH) when added to different concentrations of EPC copolymer at 25° C. DPH absorbance peaks are observed at 344, 358 and 376 nm and absorbance increase with higher concentrations in accordance with various embodiments disclosed herein.

FIG. 6 shows the absorbance spectra of 1,6-diphenyl-1,3,5-hexatriene (DPH) when added to different concentrations of EPC copolymer at 37° C. in accordance with various embodiments disclosed herein. DPH absorbance peaks are observed at 344, 358 and 376 nm and absorbance increase with higher concentrations.

FIG. 7 shows critical micelle concentration of nEPCs in accordance with various embodiments disclosed herein. Critical micelle concentration (CMC) values of EPC were determined using a dye solubilisation method where changes in absorbance of hydrophobic dye 1,6-diphenyl-1,3,5-hexatriene (DPH) was monitored. The CMC for nanomicelle formation was found to be 0.070 wt % at 25° C.

FIG. 8 to FIG. 9 show characterization of EPC nanomicelle (nEPC) size and morphology in accordance with various embodiments disclosed herein.

FIG. 8 shows hydrodynamic sizes of EPC nanomicelle (nEPCs), Aflibercept and Aflibercept-loaded nEPC (nEPCs+A) determined using dynamic light scattering (DLS) in accordance with various embodiments disclosed herein. Individually, nEPC and Aflibercept had a maximum hydrodynamic size of 57.9 nm and 13.1 nm respectively. When EPC was mixed with Aflibercept, only one band of 64.5 nm was formed, suggesting the formation of nEPCs+A.

FIG. 9 shows TEM images of the ultrastructure of nEPCs (top row) and nEPCs+A (bottom row) in accordance with various embodiments disclosed herein. Micelle morphology is determined using Electron Transmission Microscopy (TEM), with the scale bar representing 50 nm.

FIG. 10 to FIG. 17 show nEPCs (2% wt) demonstrate intrinsic anti-angiogenic properties in in-vitro studies which may be mediated through vascular endothelial growth factor (VEGF) and platelet-derived growth factor (PDGF) pathways in accordance with various embodiments disclosed herein.

FIG. 10 shows nEPCs inhibit VEGF-dependent human umbilical vein endothelial cells (HUVEC) migration in a scratch assay: HUVECs require basal VEGF to proliferate, thus 2 controls of with and without VEGF were included. HUVECs treated with nEPCs+VEGF required the longest time to heal, suggesting a maximal inhibition of HUVEC migration in accordance with various embodiments disclosed herein.

FIG. 11 shows quantification of scratch assay (% wound recovery at various timepoints): Cells treated with both aflibercept+VEGF and nEPCs+VEGF exhibited significant slowing down of wound recovery compared to control and +VEGF alone in accordance with various embodiments disclosed herein.

FIG. 12 shows HUVEC tube formation assay: Phase contrast photomicrographs (taken at 5 hours after exposure to medium): nEPCs+VEGF was able to inhibit capillary tube formation, more than aflibercept+VEGF and VEGF alone in accordance with various embodiments disclosed herein.

FIG. 13 shows quantitative analysis of total branch length in HUVEC tube formation assay performed: nEPCs+VEGF demonstrated the lowest branching length and intervals in accordance with various embodiments disclosed herein.

FIG. 14 shows quantitative analysis of branch intervals in HUVEC tube formation assay performed: nEPCs+VEGF demonstrated the lowest branching length and intervals in accordance with various embodiments disclosed herein.

FIG. 15 shows RNA expression of key genes involved in angiogenesis in HUVECs after 24 hours of treatment were measured by qPCR for VEGF A-C in accordance with various embodiments disclosed herein. Expression of VEGF-C, VEGFR1 and PDGFR-ß were significantly different in HUVECs treated with nEPCs+VEGF compared to those treated with aflibercept+VEGF. Expression of VEGF-C, VEGFR3, PDGFB and PDGFR-α were significantly different in HUVECs treated with nEPC+VEGF compared to those treated with VEGF alone. Values are expressed as mean±SD, n≥3. ****p<0.0001; ***p<0.0002; **p<0.002, *p<0.0332 versus +VEGF control.

FIG. 16 shows RNA expression of key genes involved in angiogenesis in HUVECs after 24 hours of treatment were measured by qPCR for VEGFR 1-3 in accordance with various embodiments disclosed herein. Expression of VEGF-C, VEGFR1 and PDGFR-ß were significantly different in HUVECs treated with nEPCs+VEGF compared to those treated with aflibercept+VEGF. Expression of VEGF-C, VEGFR3, PDGFB and PDGFR-α were significantly different in HUVECs treated with nEPC+VEGF compared to those treated with VEGF alone. Values are expressed as mean±SD, n≥3. ****p<0.0001; ***p<0.0002; **p<0.002, *p<0.0332 versus +VEGF control.

FIG. 17 shows RNA expression of key genes involved in angiogenesis in HUVECs after 24 hours of treatment were measured by qPCR for PDGF signalling molecules in accordance with various embodiments disclosed herein. Expression of VEGF-C, VEGFR1 and PDGFR-ß were significantly different in HUVECs treated with nEPCs+VEGF compared to those treated with aflibercept+VEGF. Expression of VEGF-C, VEGFR3, PDGFB and PDGFR-α were significantly different in HUVECs treated with nEPC+VEGF compared to those treated with VEGF alone. Values are expressed as mean±SD, n≥3. ****p<0.0001; ***p<0.0002; **p<0.002, *p<0.0332 versus +VEGF control.

FIG. 18 shows HUVEC proliferation assay: nEPCs+VEGF demonstrated a greater inhibitory effect on HUVEC proliferation compared to aflibercept+VEGF and VEGF alone in accordance with various embodiments disclosed herein. Values are expressed as mean±SD, n≥3. ****p<0.0001; ***p<0.0002; **p<0.002, *p<0.0332 versus +VEGF control.

FIG. 19 to FIG. 21 show nEPCs (2 wt %) demonstrate anti-angiogenic effect on an 3D AIM Chip in accordance with various embodiments disclosed herein.

FIG. 19 shows a schematic diagram 1900 of AIM Chip for allowing the HUVECs sprouting in a 3D environment in accordance with various embodiments disclosed herein. The device comprises left microchannel 1902, right microchannel 1904 and a middle channel 1906. The left and right microchannels are coated with fibronectin. The left fibronectin-coated lateral fluidic channel 1902 is then seeded with HUVECs 1908. The right fibronectin-coated lateral fluidic channel 1904 is empty. The in-vitro anti-angiogenic assay was performed using an AIM 3D chip with collagen type I gel 1910 in the middle channel 1906 of the device. Middle channel 1906 is filled with collagen type I gel 1910.

FIG. 20 shows confocal microscopy images of HUVEC AIM CHIP: aflibercept+VEGF treated HUVEC demonstrated greater inhibition of branching compared to nEPCs+VEGF and VEGF alone in accordance with various embodiments disclosed herein. Scale bar represents 100 μm.

FIG. 21 shows quantification of the total branch length formed by HUVECs after 5 days culture (left axis), and total cell number (lined (---) graph) counted within the area of one triangle in the AIM CHIP (right axis) in accordance with various embodiments disclosed herein.

FIG. 22A, FIG. 22B, FIG. 23 to FIG. 26 show nEPCs (2 wt %) demonstrate anti-angiogenic effect on an ex-vivo murine choroidal assay in accordance with various embodiments disclosed herein.

FIG. 22A shows experimental design of choroidal explant sprouting and regression assay in accordance with various embodiments disclosed herein. In the figure, 2D represents 2 days, 3D represents 3 days and 4D represents 4 days.

FIG. 22B shows data analysis of choroidal explant sprouting and regression assay in accordance with various embodiments disclosed herein. The quantification of choroidal sprouting area used a previously published SWIFT-Choroid method according to Shao, Z. et al., PLoS One 2013, 8, e69552, the contents of which are fully incorporated herein by reference, showing (1) original brightfield image; (2) computer-generated image after removal of central explant; and (3) final SWIFT-Choroid image. Scale bar represents 100 μm.

FIG. 23 shows sprouting regression assay (with vessel sprouting established prior to treatment): nEPCs+VEGF did not result in regression of pre-sprouted vessels but was able to inhibit further vessel sprouting in accordance with various embodiments disclosed herein.

FIG. 24 shows sprouting inhibition assay with VEGF alone, aflibercept+VEGF and nEPCs+VEGF (after 48 hours of culture): Explant exposed to nEPCs+VEGF generated fewer sprouts compared to aflibercept+VEGF and nEPC+VEGF in accordance with various embodiments disclosed herein.

FIG. 25 shows quantification of sprouting area for sprouting assay (% of total choroidal area) demonstrated reduced sprouting area in explants treated with nEPCs compared to aflibercept and VEGF alone (*p<0.05; **p<0.01; ***p<0.001) in accordance with various embodiments disclosed herein.

FIG. 26 shows quantification of sprouting area [(total area−initial area before treatment)/(total area)] demonstrated comparable reduction in sprouting area between nEPCs+VEGF and aflibercept+VEGF after 72 hours (*p<0.05; **p<0.01; ***p<0.001) in accordance with various embodiments disclosed herein.

FIG. 27 to FIG. 29 show nEPCs+Rho-A are taken up intracellularly by hCECs in-vitro in accordance with various embodiments disclosed herein.

FIG. 27 shows nEPCs+Rho-A promote in-vitro cellular uptake of aflibercept in hCECs in a concentration dependent manner (from 0-2 wt %) in accordance with various embodiments disclosed herein. Confocal images were taken after 24 hours incubation at different concentrations of nEPCs (0.05 wt %, 0.2 wt %, 1 wt % and 2 wt %). Scale bar=10 μm.

FIG. 28 shows quantitative cellular uptake results analysed by flow cytometry in hCECs after 24 hours incubation with nEPCs+A at different nEPCs concentrations in accordance with various embodiments disclosed herein.

FIG. 29 shows the cellular distribution and co-localization of fluorescein-containing EPC polymer (FEPCs) and Rho-A viewed by a confocal laser microscope (100×), z-stack imaging was taken to confirm the intracellular distribution of the complexes in accordance with various embodiments disclosed herein. Ocular penetration of aflibercept across ex-vivo porcine scleral and cornea in the presence and absence of nEPCs (2 wt %).

FIG. 30 to FIG. 31 show nEPCs+Rho-A enhances ocular penetration of Rho-A in an ex-vivo porcine scleral model in accordance with various embodiments disclosed herein.

FIG. 30 shows measurement of aflibercept penetration across excised porcine sclera using an Ussing chamber (n≥3, mean±SD) in accordance with various embodiments disclosed herein.

FIG. 31 shows measurement of aflibercept concentration within the porcine vitreous 45 min after a single eye-drop application (20 μL) directly on an porcine corneal-scleral eyecup (n=3, mean±SD) in accordance with various embodiments disclosed herein.

FIG. 32 to FIG. 33 show nEPCs+Rho-A enhance ocular penetration of Rho-A in an in-vivo murine eye model in accordance with various embodiments disclosed herein.

FIG. 32 shows in-vivo ocular distribution of aflibercept and nEPCs+Rho-A, 45 minutes after a single eye-drop application in mice in accordance with various embodiments disclosed herein. Rho-A was observed only on the corneal epithelial layer when applied directly; but nEPCs+Rho-A was able to penetrate the cornea. (White arrows refer to Rho-A).

FIG. 33 shows the amount of Rho-A which successfully penetrated the cornea to reach the mouse vitreous that was evaluated 45 minutes after the single eye-drop and compared with the nEPCs+Rho-A eye-drop in accordance with various embodiments disclosed herein. (n=10, mean±SD). (*p<0.05; **p<0.01; ***p<0.001).

FIG. 34A, FIG. 34B, FIG. 34C, FIG. 35 to FIG. 36 show comparison of Corneal Retention Time between nEPCs+RhoA and RhoA in accordance with various embodiments disclosed herein.

FIG. 34A shows anterior segment optic coherence tomography (ASOCT) imaging after 1 drop of each solution (nEPCs+Rho-A, Rho-A) was applied on eyes of mice in accordance with various embodiments disclosed herein. Photos were taken at timepoints 30 s, 60 s with manual blinking performed at regular intervals.

FIG. 34B shows anterior segment optic coherence tomography (ASOCT) imaging after 1 drop of each solution (nEPCs+Rho-A, Rho-A) was applied on eyes of mice in accordance with various embodiments disclosed herein. Photos were taken at timepoints 120 s, 210 s with manual blinking performed at regular intervals.

FIG. 34C shows anterior segment optic coherence tomography (ASOCT) imaging after 1 drop of each solution (nEPCs+Rho-A, Rho-A) was applied on eyes of mice in accordance with various embodiments disclosed herein. Photos were taken at timepoints 285 s, 300 s with manual blinking performed at regular intervals.

FIG. 35 shows the area above the cornea occupied by eyedrops quantified, using ASOCT images in accordance with various embodiments disclosed herein. nEPCs+Rho-A demonstrated a significantly greater area which persisted from 8 blinks onwards. (*p<0.05; **p<0.01; ***p<0.001).

FIG. 36 shows anterior segment photos of the mouse eye after 20 blinks from 1 drop of each eyedrop (nEPCs+Rho-A, Rho-A) delivered in accordance with various embodiments disclosed herein. The photos showed retention of nEPCs+Rho-A eyedrops on the cornea surface unlike Rho-A alone.

FIG. 37A and FIG. 37B show biocompatibility of nEPCs in-vitro in accordance with various embodiments disclosed herein.

FIG. 37A shows cell viability measured by Lactate Dehydrogenase Release (LDH) assay on hCECs and ARPE-19 cell lines in accordance with various embodiments disclosed herein.

FIG. 37B shows cell death measured by Lactate Dehydrogenase Release (LDH) assay on hCECs and ARPE-19 cell lines in accordance with various embodiments disclosed herein.

FIG. 38 shows biocompatibility of nEPCs ex-vivo. To assess the effect of nEPCs on the integrity of porcine corneal tissue, the transepithelial electrical resistance (TEER) was measured after prolonged exposure to aflibercept or nEPCs+A in accordance with various embodiments disclosed herein. Quantitative analysis showed no significant decrease in TEER for either arms after 24 hr exposure.

FIG. 39A, FIG. 39B, FIG. 39C, FIG. 39D, FIG. 39E show biocompatibility of nEPCs in-vivo in accordance with various embodiments disclosed herein. The in-vivo biocompatibility of nEPCs (2 wt %) & nEPCs+A were monitored using a mice model after 14 days of daily topical eye-drop (5 μL each time, thrice a day). Among all treatment arms, slit lamp imaging (undilated) did not reveal any cornea opacities, (dilated) did not reveal cataract formation. Histology shows preservation of cornea architecture, ZO-1 immunofluorescent staining highlighted the maintenance of corneal epithelium tight-junction integrity and TUNEL stain did not show any increase in apoptosis. Scale bar=50 μm. (Epi: Corneal epithelial layer; Endo: Corneal endothelial layer).

FIG. 40 to FIG. 42 show topical application of nEPCs+A causes CNV regression in laser-induced mice model in accordance with various embodiments disclosed herein. Topically instilled nEPCs+A significantly reduced the leakage area of laser induced CNV in mice (n=8).

FIG. 40 shows fundus fluorescein angiography (FFA) images taken from a representative eye on 3^{rd}, 7^{th} and 14^{th} days after model establishment in accordance with various embodiments disclosed herein.

FIG. 41 shows the fluorescence leakage degree in choroidal lesion area (n=8) quantified by ImageJ, based on the FFA images shown in FIG. 40. The daily recovery rate was calculated using following formula: (leakage area on 3^{rd} day−leakage area on 14^{th} day)/((14−3) days. *p<0.05, **p<0.01, ***p<0.001 versus nEPCs+A.

FIG. 42 shows isolectin B4 (red) staining of endothelial cells on the choroidal flat mounts indicating overall reduction in size of CNV lesions after treatment with EPCs+A (white arrow points lesion created by laser) in accordance with various embodiments disclosed herein.

FIG. 43 shows the retention time of EPC copolymer in gel permeation chromatography (GPC) using tetrahydrofuran (THF) as solvent, in accordance with various embodiments disclosed herein.

FIG. 44A shows ¹H NMR spectrum of EPC copolymer in CDCl₃ in accordance with various embodiments disclosed herein.

FIG. 44B shows the identity of the corresponding protons (a, b, c, d, e, f, g, and h) in the chemical structure shown in the ¹H NMR spectrum of EPC copolymer in FIG. 44A. The corresponding protons in the chemical structure are identified, and the integration ratios of the characteristic PEG, PPG, PCL peaks are shown in Table 2.

FIG. 45 to FIG. 47 show characterization of fluorescein-diol in accordance with various embodiments disclosed herein.

FIG. 45 shows ¹H NMR spectra of fluorescein-diol in CDCl₃ in accordance with various embodiments disclosed herein.

FIG. 46 shows ¹³C NMR spectra of fluorescein-diol in CDCl₃ in accordance with various embodiments disclosed herein.

FIG. 47 shows 2D ¹H-¹H COSY spectra of fluorescein-diol in CDCl₃ in accordance with various embodiments disclosed herein.

FIG. 48 to FIG. 50 show characterization of FEPC in accordance with various embodiments disclosed herein.

FIG. 48 shows ¹H NMR of FEPC polyurethane in CDCl₃ in accordance with various embodiments disclosed herein. Inset shows expanded aromatic region with peaks corresponding to fluorescein aromatic groups.

FIG. 49 shows GPC trace (THF) of the FEPC thermogelling polymer in accordance with various embodiments disclosed herein.

FIG. 50 shows critical micelle concentration (CMC) values of FEPC determined using a dye solubilisation method at 37° C. where changes in absorbance of hydrophobic dye 1,6-diphenyl-1,3,5-hexatriene (DPH) was monitored in accordance with various embodiments disclosed herein.

FIG. 51 to FIG. 53 show characterization and evaluation of commercial F127.

FIG. 51 shows CMC values of F127 measured at 25° C. and 37° C., as compared with those of EPC. In the figure, squares (▪) represent F127 and circles (●) represent EPC.

FIG. 52 shows quantitative cellular uptake results analysed by flow cytometry in hCECs after 24 hours incubation, FITC-A compared with nEPCs+FITC-A and F127+FITC-A in accordance with various embodiments disclosed herein.

FIG. 53 shows topically instilled Rho-A complexes F127 (F127-Rho-A) in-vivo demonstrated its poor ability for corneal penetration in the murine eye.

### EXAMPLES

Example embodiments of the disclosure will be better understood and readily apparent to one of ordinary skill in the art from the following examples, tables and if applicable, in conjunction with the figures. It should be appreciated that other modifications related to structural, biological and/or chemical changes may be made without deviating from the scope of the invention. Example embodiments are not necessarily mutually exclusive as some may be combined with one or more embodiments to form new example embodiments. The example embodiments should not be construed as limiting the scope of the disclosure.

The following examples describe a nanomicelle drug delivery system made of a co-polymer which is capable of delivering bioactive/drug to the posterior segment topically through corneal-scleral routes. In the following examples, EPC is used as the co-polymer which is comprised of polyethylene glycol (PEG), polypropylglycol (PPG) and polycaprolactone (PCL) segments; and the bioactive/drug used is aflibercept.

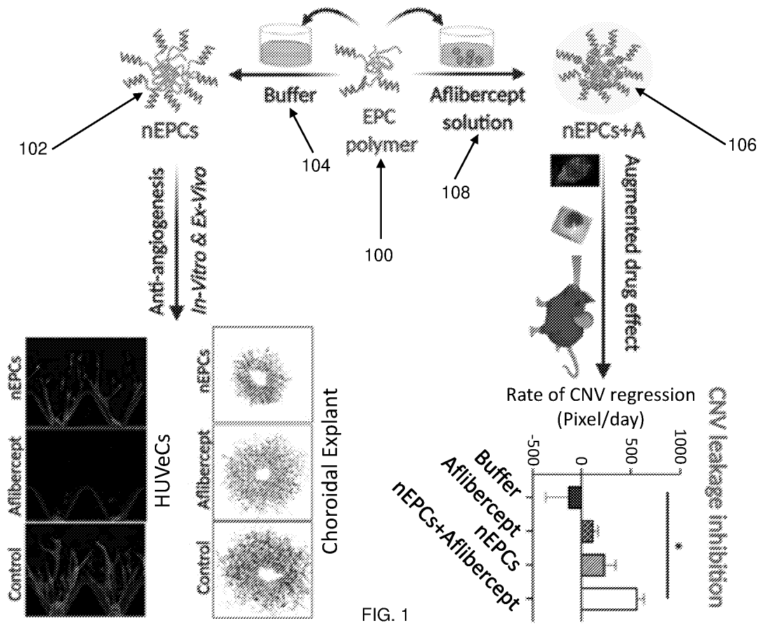

FIG. 1 shows a schematic diagram of a multi-block copolymer (e.g., EPC polymer 100) in accordance with various embodiments disclosed herein. As shown in FIG. 1, the EPC polymer 100 is self-assembled into micelles (e.g., polymeric nanomicelles (nEPCs) 102). nEPCs 102 are produced by concentrating EPC polymer 100 above the critical micelle concentration (CMC) but lower than the concentration required for sol-gel transition.

Advantageously, nEPCs 102 alone are able to inhibit angiogenesis in-vitro and ex-vivo. nEPCs possess intrinsic anti-angiogenic activity which synergizes with its drug delivery capability for the treatment of neovascular retinal diseases.

As shown in FIG. 1, aflibercept-loaded nanomicelles (i.e. nEPCs+A 106) can be formed by encapsulating aflibercept in EPC co-polymer solution. Aflibercept 104 is encapsulated by nEPCs through direct mixing to form nEPC+aflibercept (nEPCs+A) complexes 106. Aflibercept is chosen because it has a relative longer duration of effect (i.e., it is FDA-approved for a longer dosing of up to a 3-month interval), compared to monthly ranibizumab or bevacizumab. When administered topically on the murine cornea, nEPCs functioned as a drug carrier to deliver aflibercept across the cornea to achieve therapeutic concentrations in the retina of laser-induced disease models of CNV. nEPCs+A are capable of delivering clinically significant amounts of aflibercept to the retina for control of choroidal neovascularization in mice.

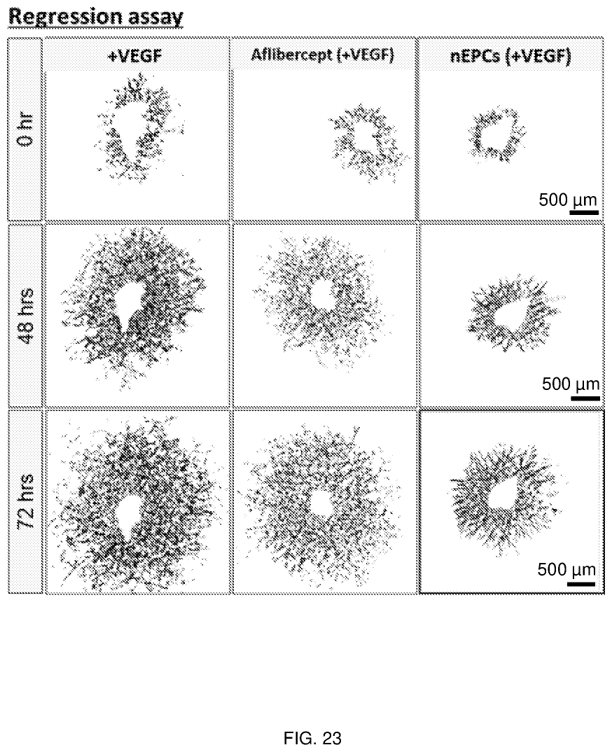

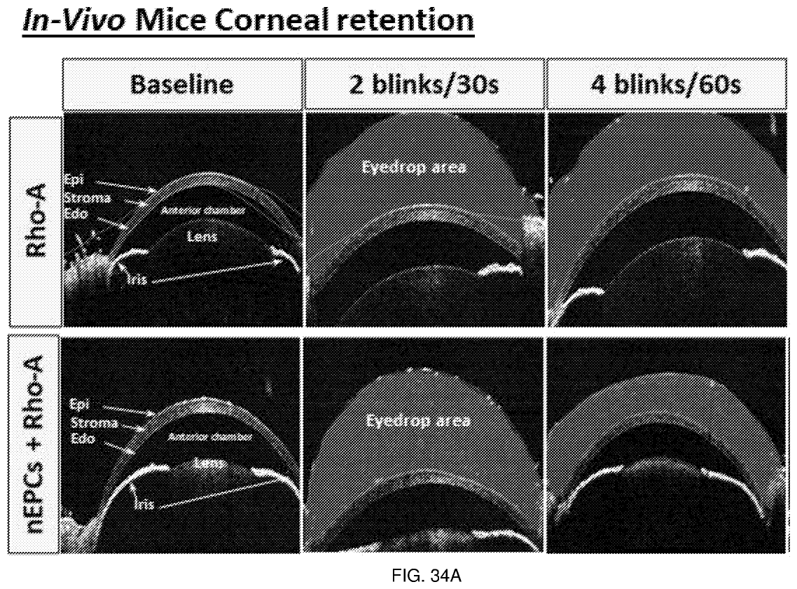

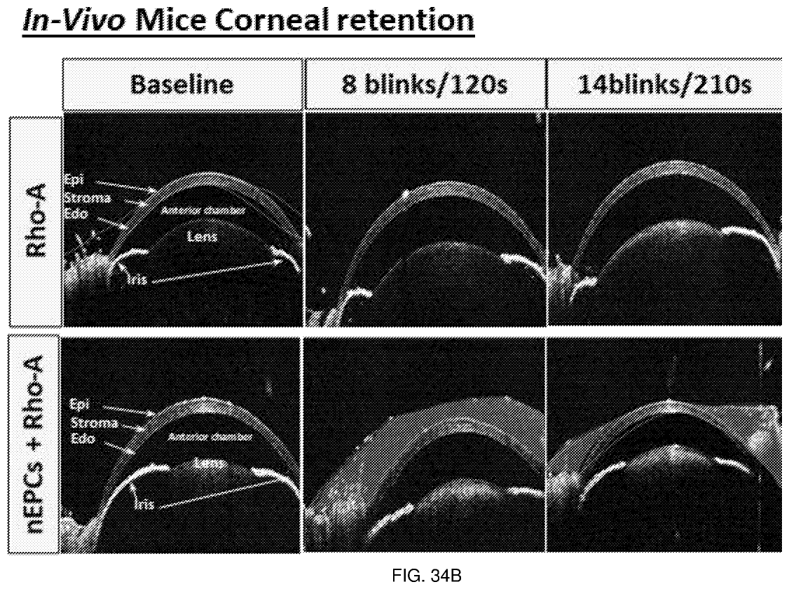

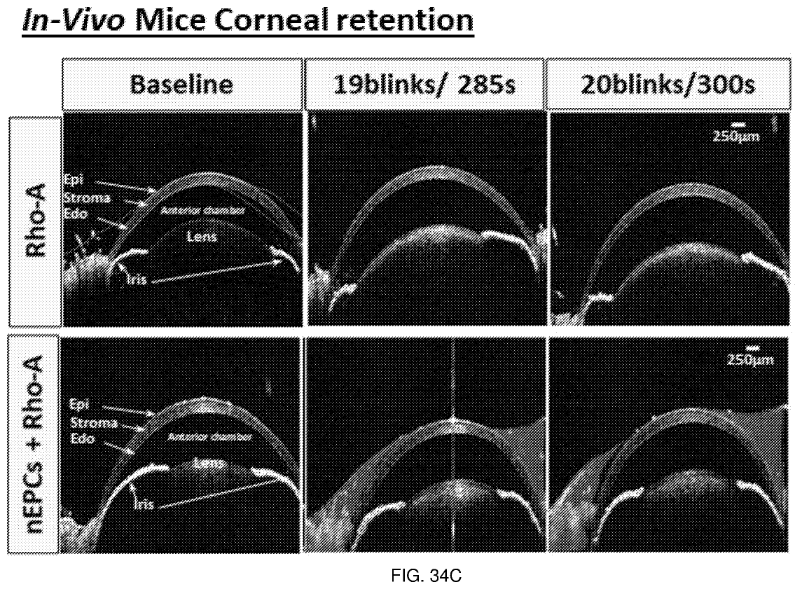

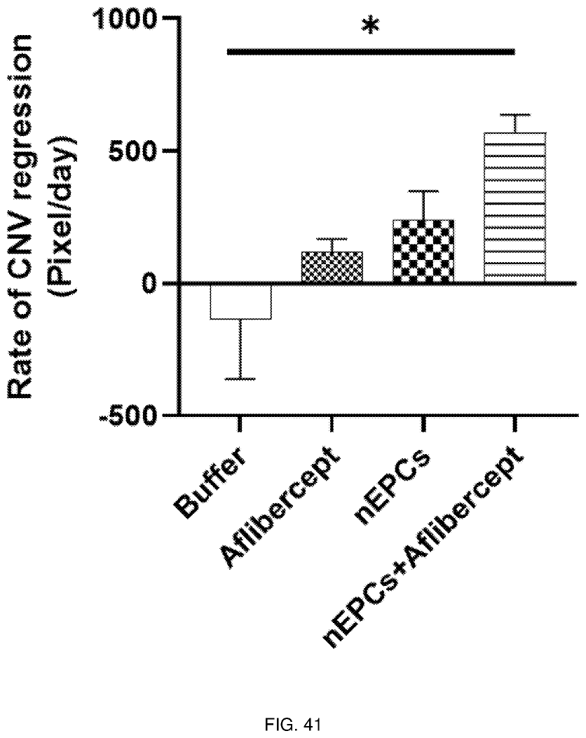

As will be shown in the following examples, aflibercept-loaded nEPCs (nEPCs+A) are capable of penetrating the cornea in ex-vivo porcine eye models and deliver a clinically significant amount of aflibercept to the retina of laser-induced choroidal neovascularisation (CNV) murine models, causing CNV regression (see e.g., FIG. 34A, 34B, 34C and FIG. 41). nEPCs+A also demonstrates biocompatibility in-vitro and in-vivo (see e.g., FIG. 20 and FIG. 23). The ability to deliver anti-VEGF drugs and the intrinsic anti-angiogenic properties of nEPCs can work synergistically and can be harnessed for effective therapeutics. nEPCs have shown to be a promising topical anti-VEGF delivery platform for the treatment of retinal diseases.

### Example 1: Development and Characterization of nEPCs+A

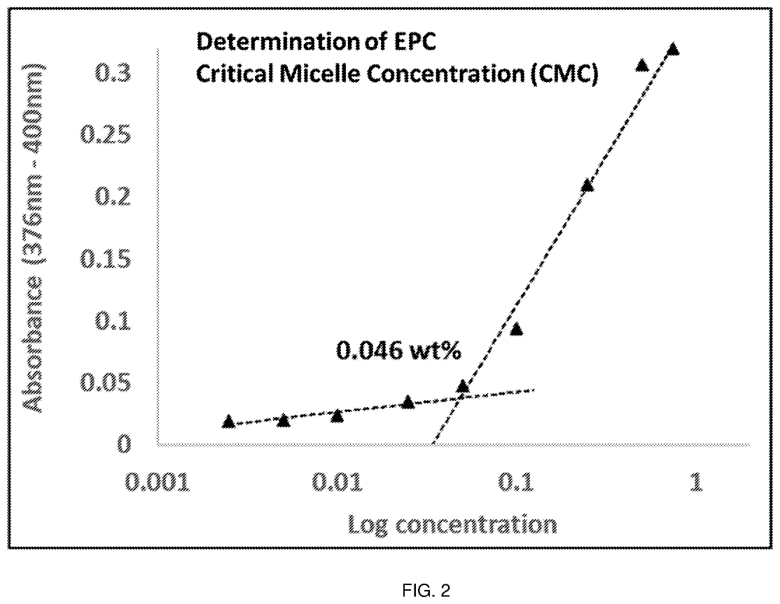

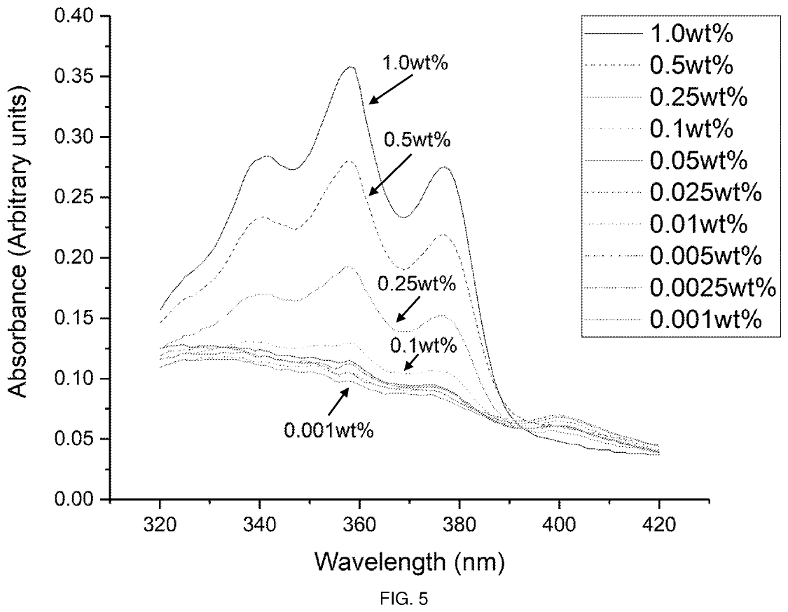

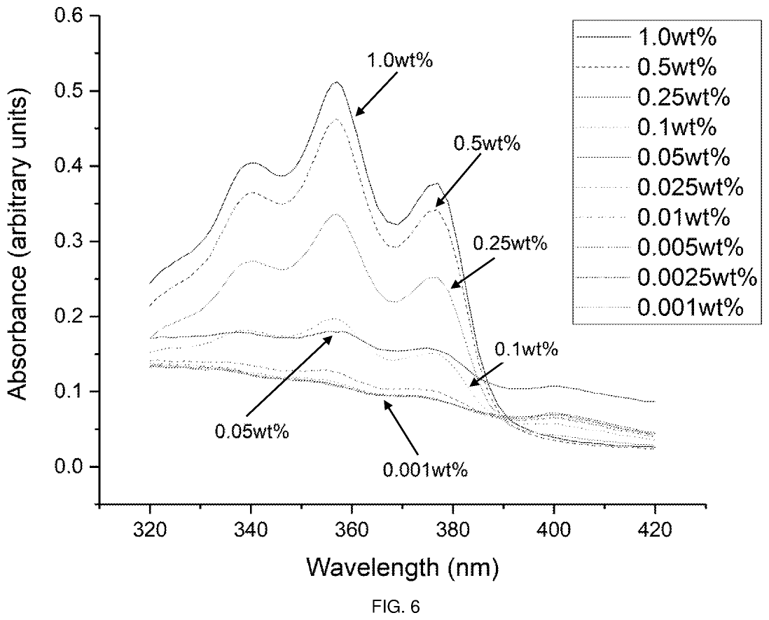

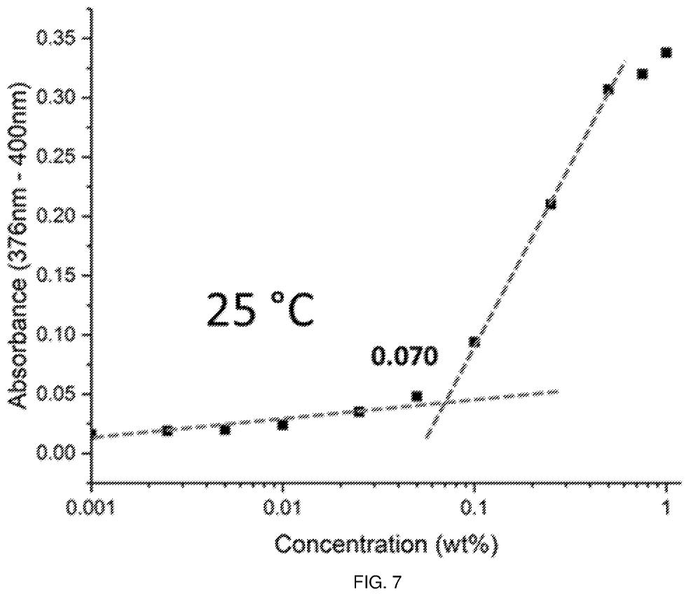

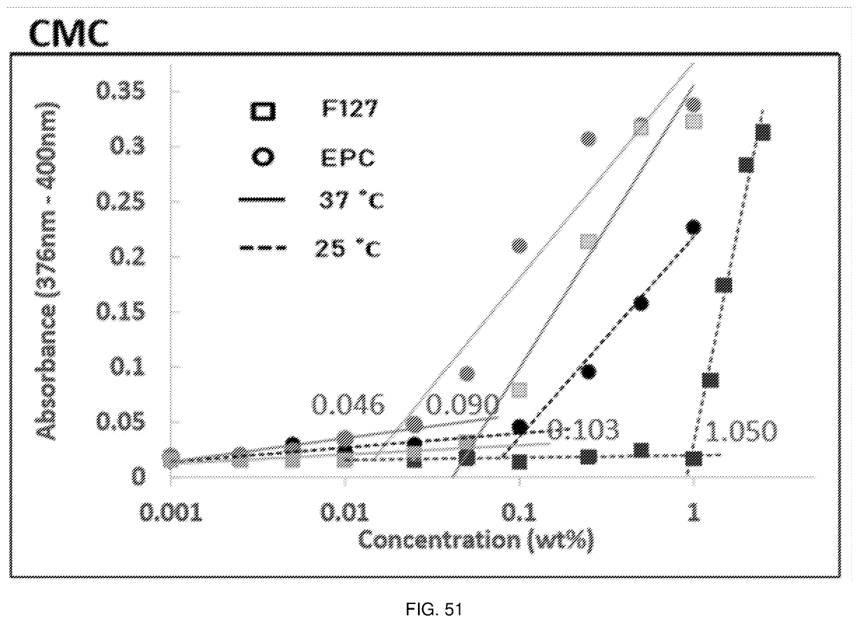

The thermodynamic self-assembly process of EPC copolymers into micelles can be described by the CMC, or minimum concentration of polymer required for micelles to form. The CMC was measured by monitoring the sharp increase in absorbance of hydrophobic dye 1,6-diphenyl-1,3,5-hexatriene (DPH) upon micelle formation (FIG. 2 & FIG. 5 to 7). CMC values for nEPCs formation was found to be 0.046 wt % at 37° C. (FIG. 2). A comparison of CMC values with Pluronic F127 (0.09 wt %) was also made. Pluronic F127 is a FDA-approved polymer which has been widely used in drug delivery and controlled release of drugs. In comparison, the CMC value for nEPCs formation was lower as compared to Pluronic F127 (0.09 wt %) (FIG. 2, FIG. 51).

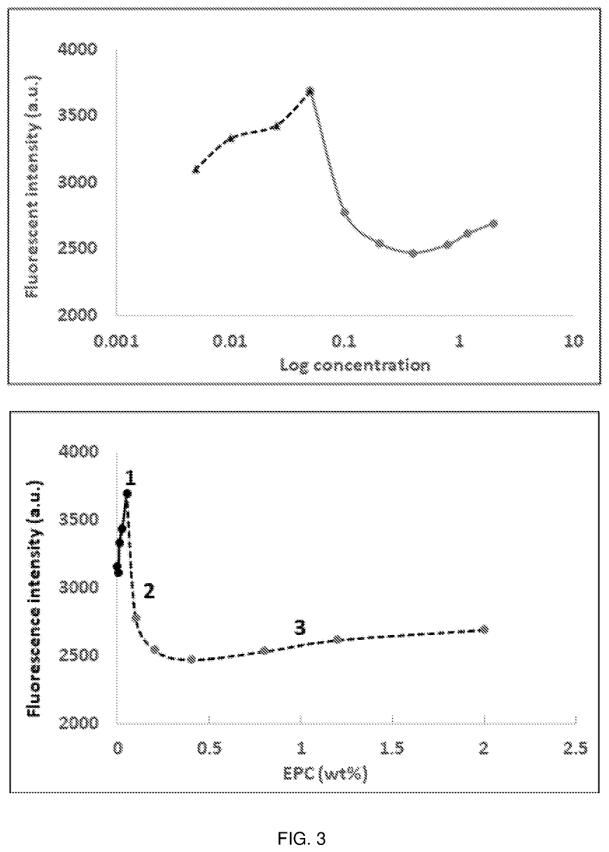

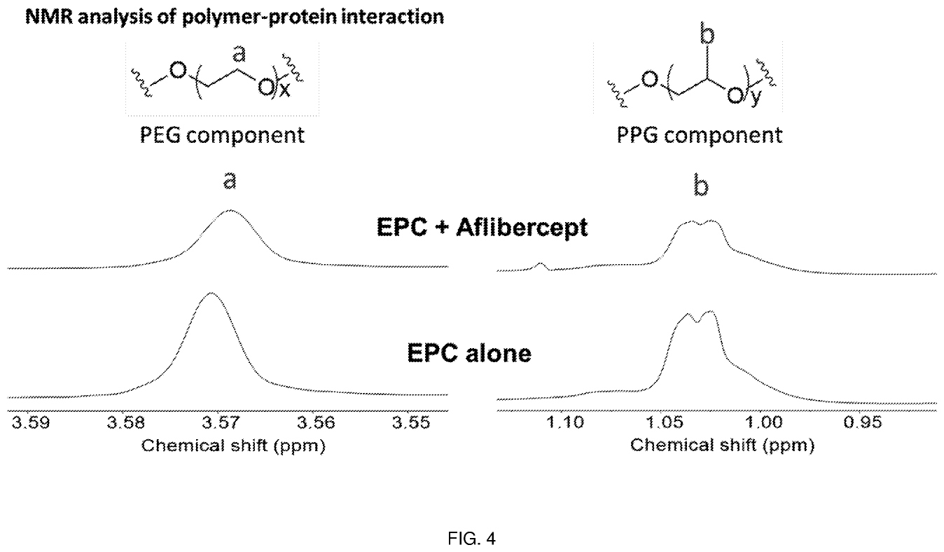

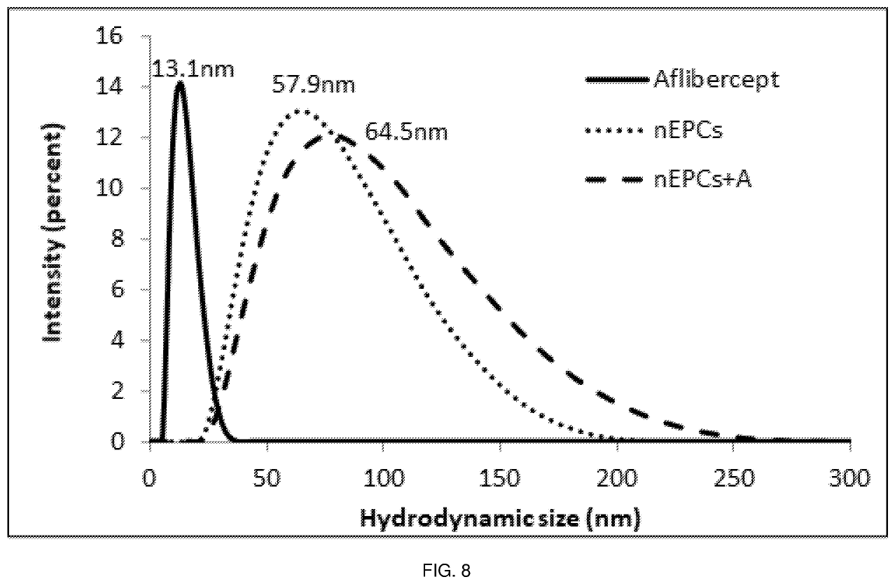

To determine the ability of nEPCs to act as a drug delivery system for aflibercept, nEPCs+A was first generated by dissolving EPC in a stock solution of aflibercept at a concentration higher than the CMC but lower than the sol-gel transition concentration. Under this condition, the EPC co-polymer self-assembled into nEPCs+A. The formation of nEPCs+A was observed by monitoring the hydrodynamic size of nEPCs at 0.2 wt % and aflibercept. Individually, nEPCs and aflibercept had a maximum hydrodynamic size of 57.9 and 13.1 nm respectively. When EPC was mixed with aflibercept solution to achieve the CMC, the 2 size distribution bands merged into 1 band and shifted to 64.5 nm, suggesting that nEPCs+A was formed (FIG. 8). The formation of nEPCs+A was studied using a fluorescent assay, in which a fixed amount of fluorescent Rhodamine-conjugated Aflibercept (Rho-A) was added into EPC solutions of various concentrations (FIG. 3). The fluorescence intensity emitted by free Rho-A was high initially and there was a sharp reduction in fluorescence intensity at 0.05 wt % EPC. This reduction in fluorescence intensity occurred at a similar polymer concentration as the CMC of EPC (CMC_{EPC}=0.046 wt %), suggesting that when nEPCs were formed, the free Rho-A was incorporated into the nEPC structure, forming nEPCs+A. Rhodamine shows a higher fluorescence in aqueous environments, so it is plausible that Rho-A was encapsulated into the micelle. To further study the micelle-drug interaction, the ¹H NMR spectra of EPC with and without aflibercept were compared (FIG. 4). The presence of aflibercept elicited a noticeable upfield shift of the PEG protons, which suggested modulation of its hydration environment due to non-covalent interactions with the drug. This is likely due to the ion-dipole interactions from the Lewis basic oxygen atoms on PEG and the aflibercept, which is cationic at physiological pH. Furthermore, aflibercept resulted in broadening of the ¹H NMR resonances arising from both the PEG and PPG segments of EPC, which is consistent with reduced chain motion resulting from aflibercept association with the polymeric micelles.

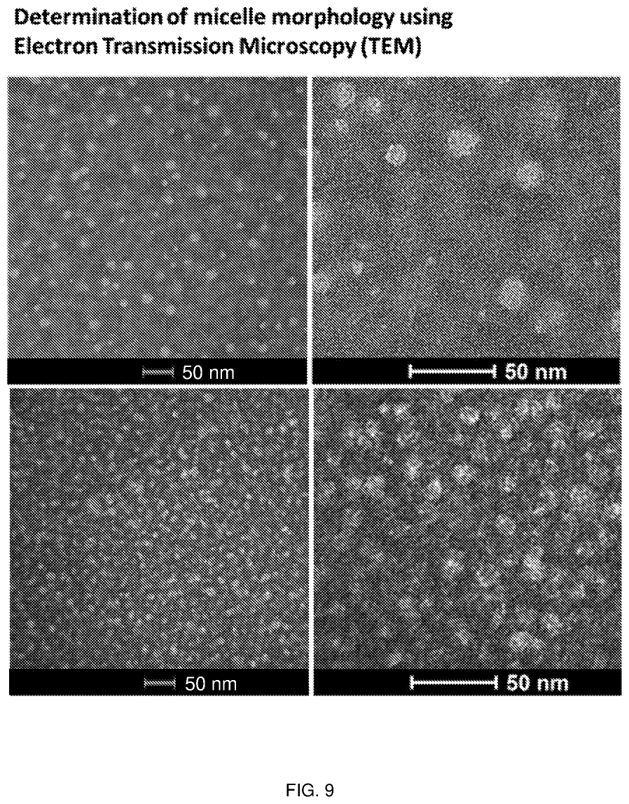

The size of nEPCs and nEPCs+A was observed by monitoring the hydrodynamic size of nEPCs at 0.2 wt % and aflibercept. Individually, nEPCs and aflibercept had a maximum hydrodynamic size of 57.9 and 13.1 nm respectively. When EPC was mixed with aflibercept solution to achieve the CMC, the 2 size distribution bands merged into 1 band and shifted to 64.5 nm, suggesting that nEPCs+A was formed (FIG. 8). nEPCs and nEPCs+A were also studied by transmission electron microscopy (TEM), and shown to form into mostly spherically shaped particles of approximately similar size. The particles in TEM were smaller in size than DLS, possibly due to air drying and collapse of the PEG shell of the micelles (FIG. 9).

To determine the maximal encapsulation efficiency (EE) of nEPCs, various concentrations of nEPCs were studied (0.5, 1.0 and 2.0 wt %). As nEPCs concentration increased, the respective EE of aflibercept were 1.3±0.4 for 0.5 wt %, 17.4±0.3 for 1.0 wt %, 47.3±0.8% for 2.0 wt % respectively. Subsequent experiments were performed using nEPC+A with 2 wt % EPC for maximal EE. For subsequent experiments utilising nEPCs, 2 wt % concentration was utilised.

### Example 2: nEPCs Intrinsically Inhibit VEGF-Induced Endothelial Cell Migration, Proliferation, Tube Formation In-Vitro and Ex-Vivo

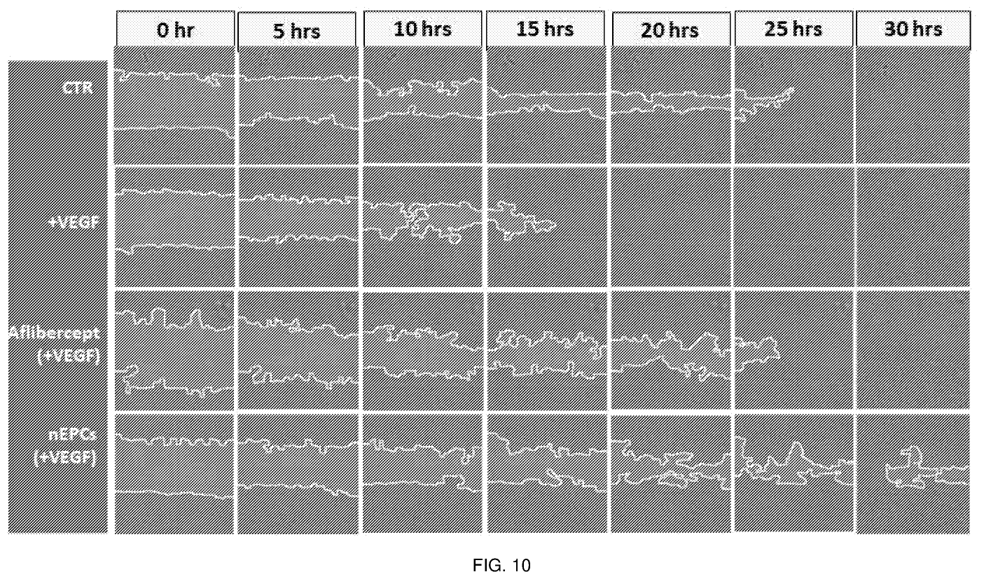

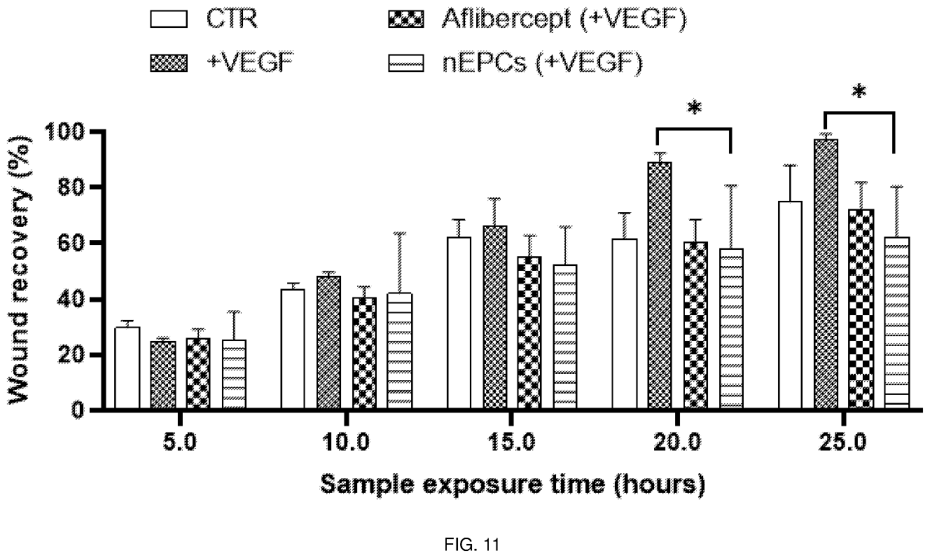

To study the anti-angiogenic properties of nEPCs, both in-vitro and ex-vivo assessments were performed. In-vitro methods include the HUVEC migration, proliferation and tube formation assays. In the HUVEC migration assay, 30 hours were required for complete wound healing in the control experiment. When VEGF was added, wound healing was accelerated to 20 hours. The addition of aflibercept to a VEGF-treated experiment demonstrated inhibition of VEGF effects. By 25 hours, the wound closure was incomplete with only 76.0±11.3% closure achieved. Interestingly, the addition of nEPCs to a VEGF-treated experiment also demonstrated a similar slower wound closure process. By 25 hours, only 67.7±18.4% wound closure was achieved. This result suggests that nEPC alone was able to inhibit VEGF-induced HUVEC migration (FIG. 10 and FIG. 11).

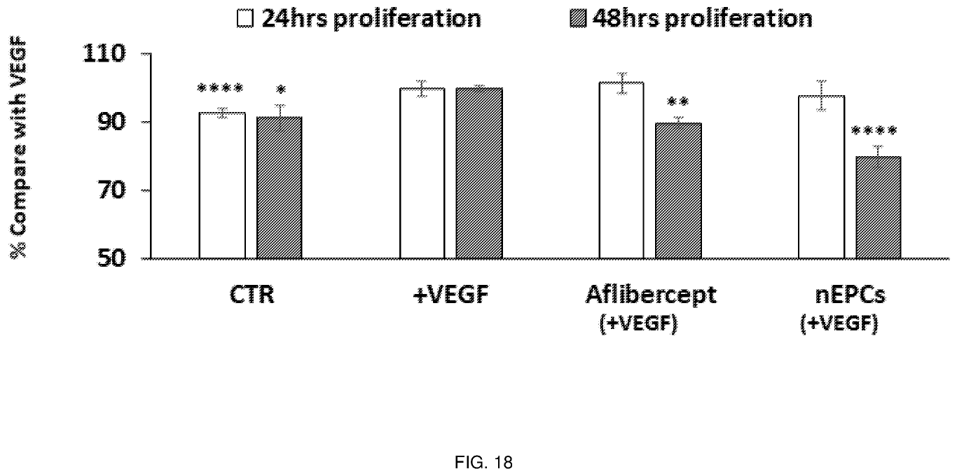

In addition, in a HUVEC proliferation assay, the addition of nEPCs was also able to reduce HUVEC proliferation. After 48 hours of incubation, the addition of aflibercept alone resulted in 88.2±4.4% cells, whilst addition of nEPC alone was also able to reduce HUVEC proliferation, with 79.8±3.1% cells after 48 hours (FIG. 18).

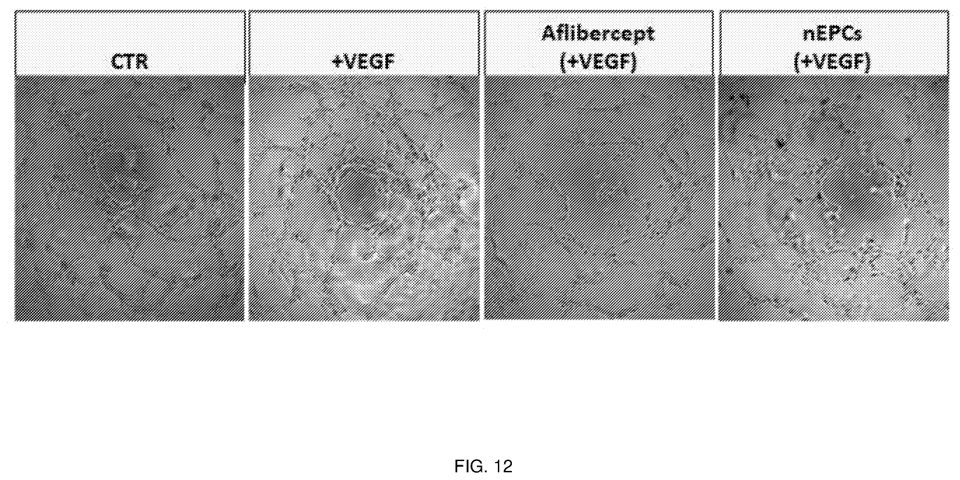

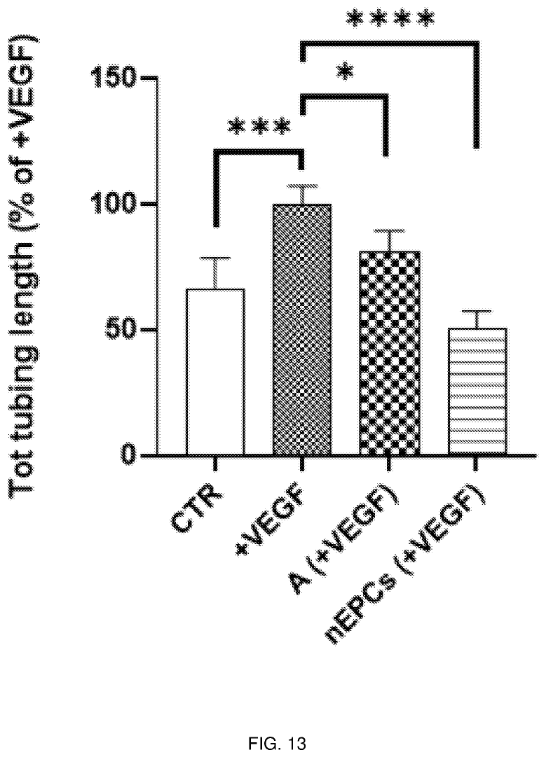

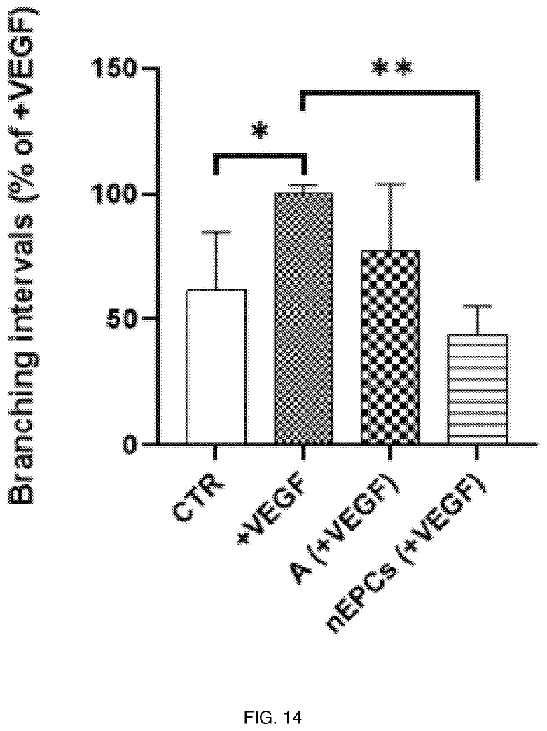

Furthermore, nEPCs were also able to significantly inhibit tube formation in terms of both branching length and branching intervals in the HUVEC tube formation assay (FIG. 12). nEPCs alone was able to reduce tube length formation (62.8%±9.0) and branching intervals (43.9%±11.4), more so than aflibercept alone (i.e. tube length formation of 81.0%±10.4 and branching intervals of 77.4%±26.4) (FIG. 13 and FIG. 14).

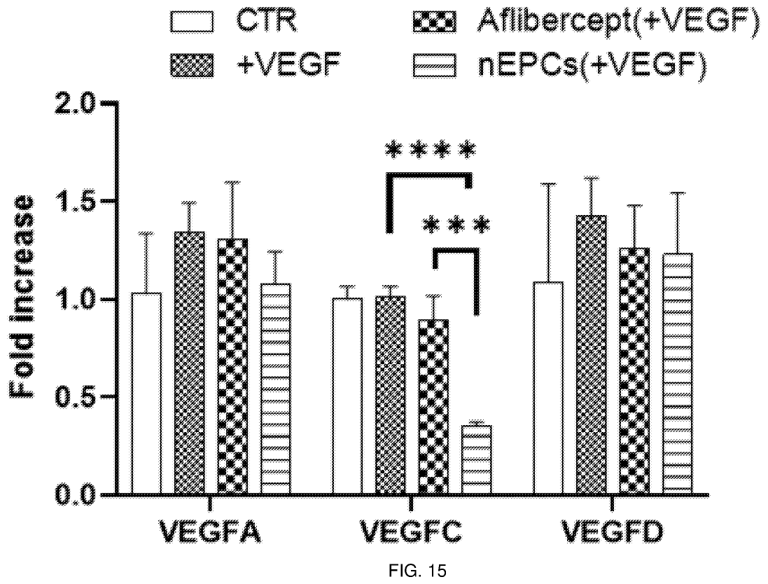

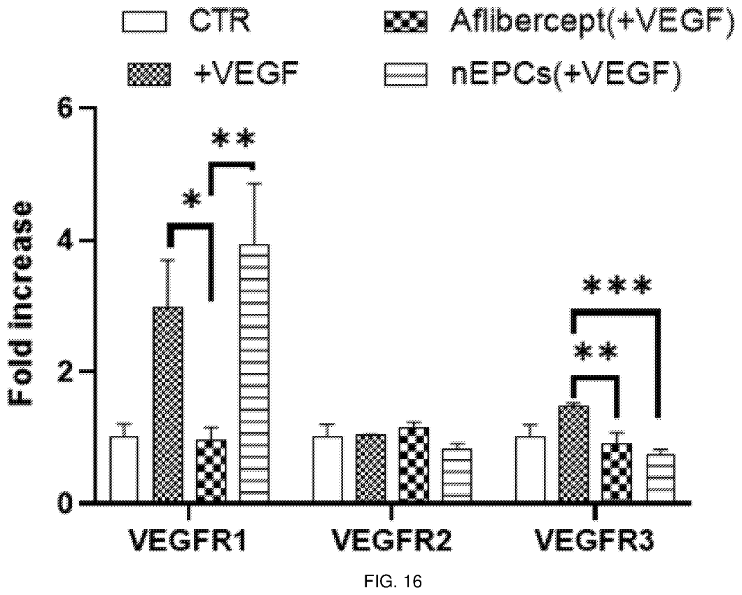

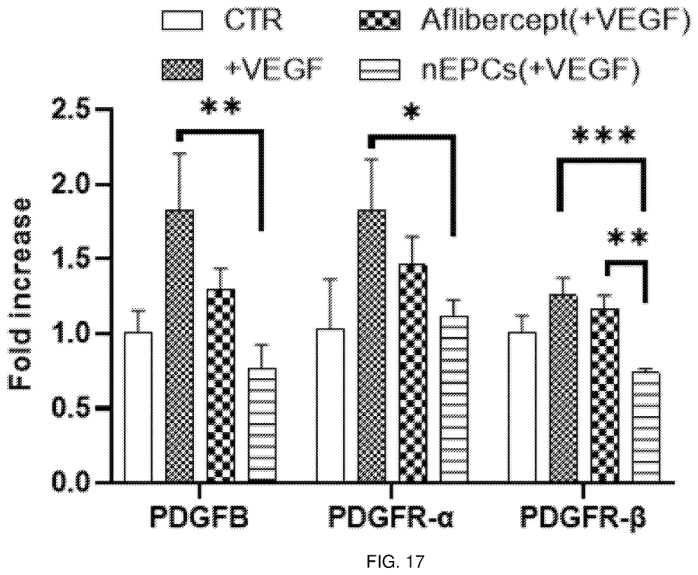

To elucidate the anti-angiogenic mechanisms of nEPCs, RNA expression of angiogenesis genes in HUVECs were assessed using qPCR (FIG. 15, FIG. 16 and FIG. 17). The RNA expression of various isoforms of VEGF (FIG. 15) and their receptors (VEGFR) (FIG. 16), which are known to play important roles in retinal neovascularisation, were assessed. nEPCs alone was able to significantly reduce the expression of VEGF-C and VEGFR3 in contrast to aflibercept, which downregulates primarily VEGFR1 expression. Upregulation of platelet-derived growth factor (PDGF) is known to confer anti-VEGF resistance. Interestingly, nEPCs alone was capable of significantly reducing expression of PDGFB, PDGFR-α, PDGFR-ß compared to aflibercept. These results suggest that the anti-angiogenic effects of nEPCs occur through both VEGF and non-VEGF mediated pathways. Importantly, they seem to be distinct from, but yet synergistic with aflibercept dependent pathways.

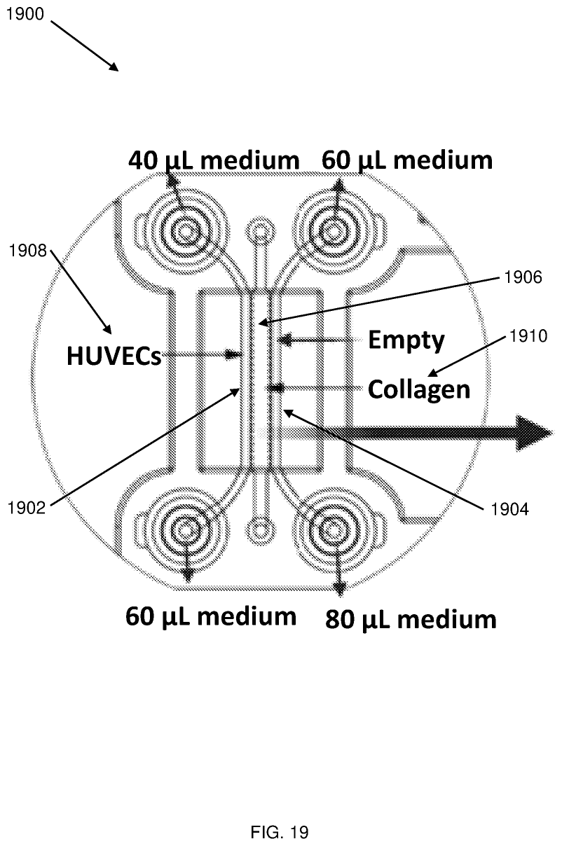

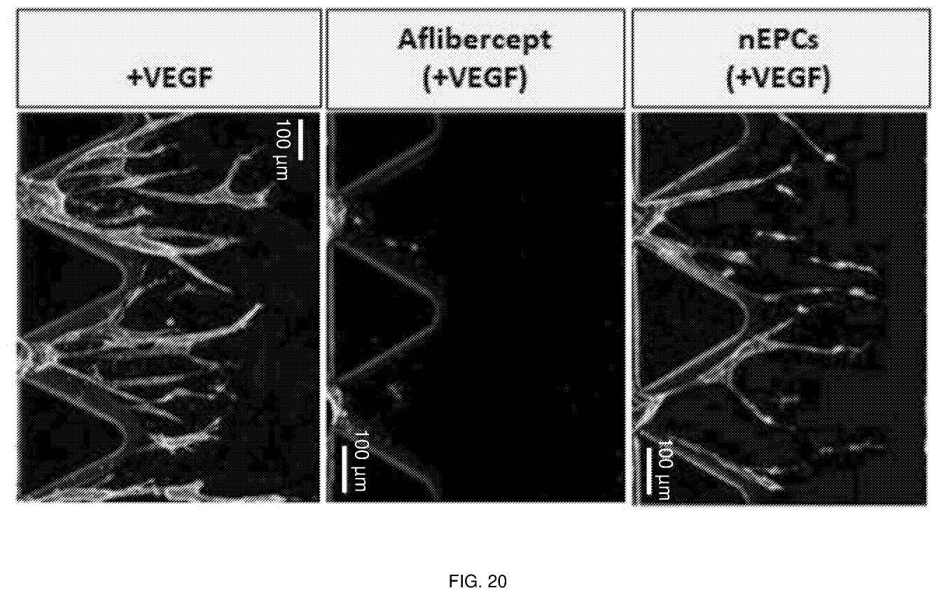

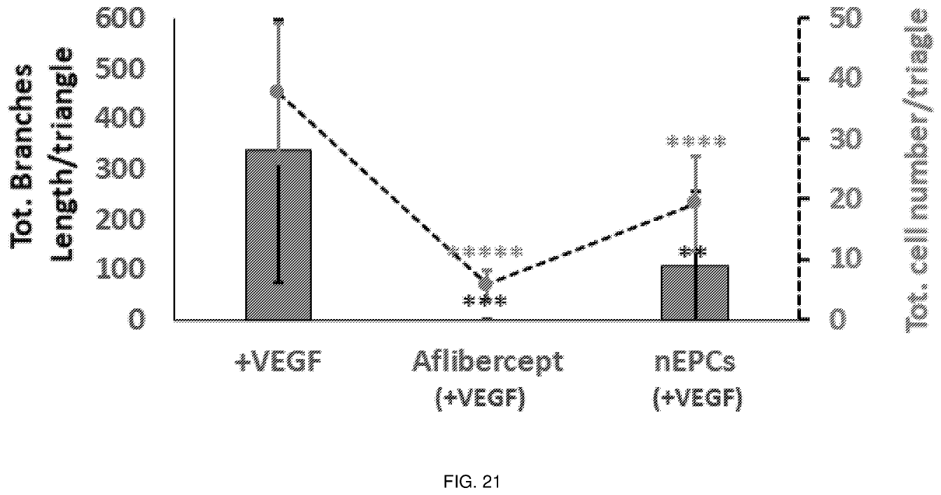

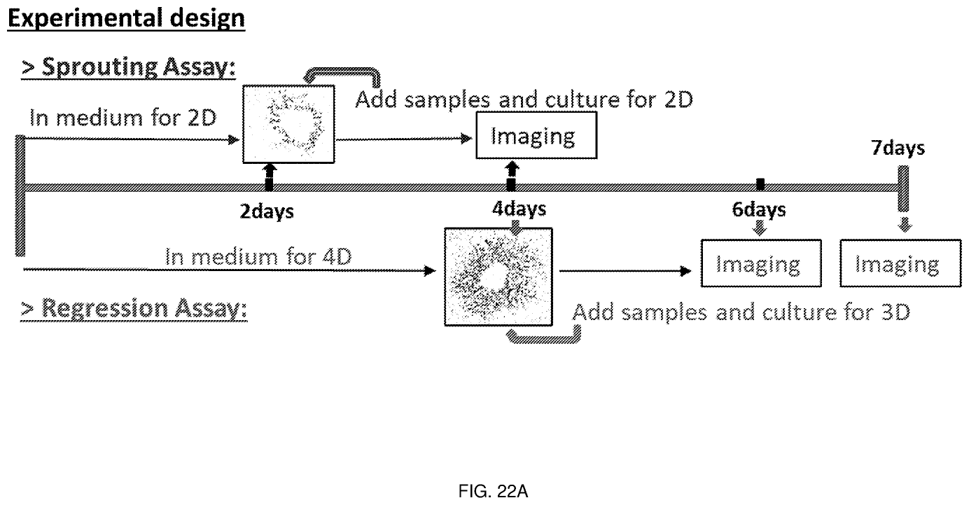

To further characterise the ability of nEPCs to inhibit angiogenesis, a 3D cell model was utilised to assess HUVEC migration and tube formation in parallel. In this single assay, HUVECs sprouted and migrated from a pre-existing monolayer into the connected 3D collagen matrix providing a concentration gradient of angiogenic stimuli (FIG. 19). In the VEGF control, HUVECs migrated into the central collagen channel and formed tubular structures after 5 days in culture. Both migration and tube formation were significantly inhibited in the presence of aflibercept with minimal cell migration into the collagen channel. Although nEPCs did not inhibit HUVEC migration into the central collagen channel (FIG. 20), the total branch length was reduced from 336.0 pixel (in +VEGF controls) to 106.7 pixel (p=0.0087) (FIG. 21).

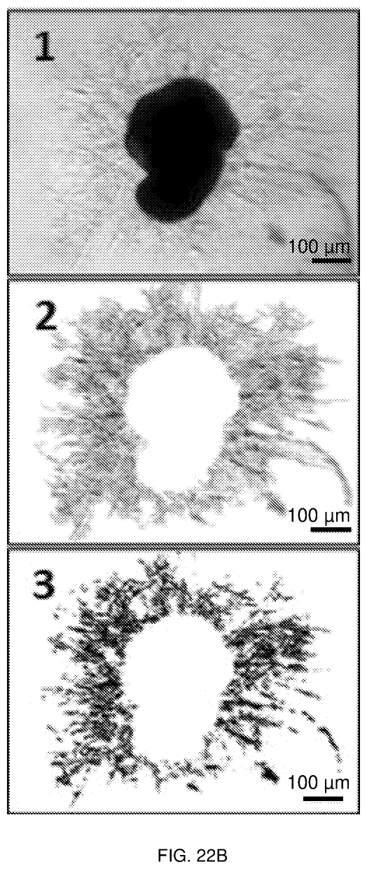

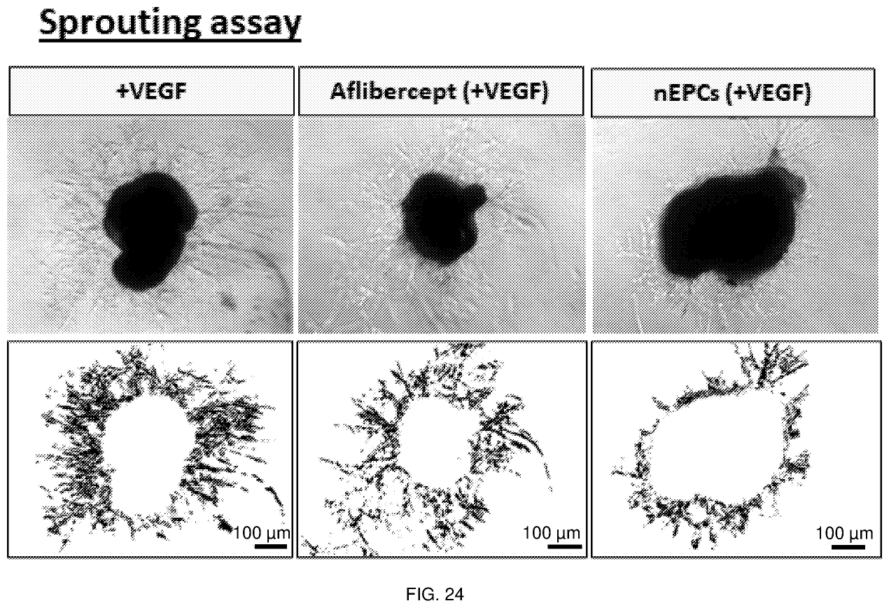

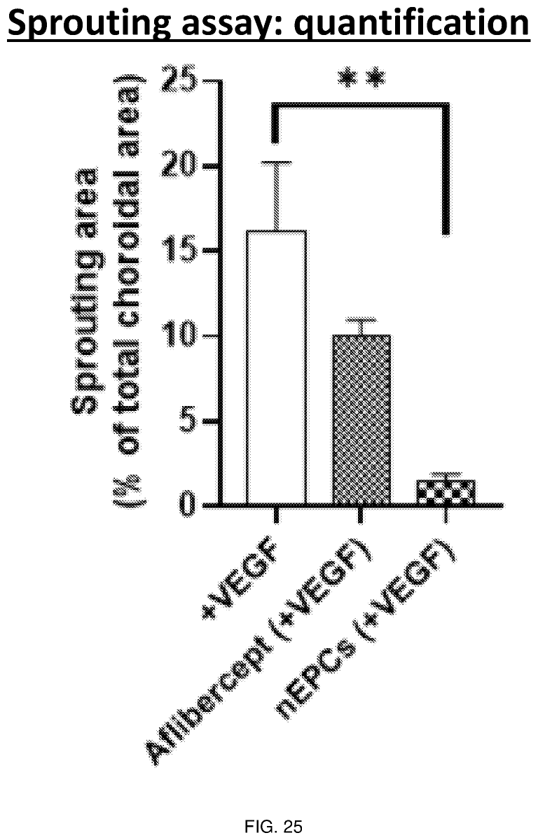

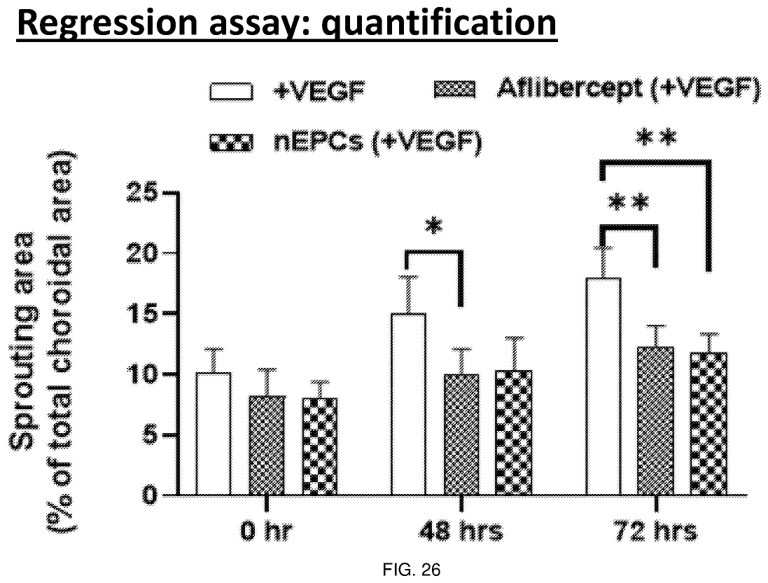

To further evaluate the ability of nEPCs to inhibit angiogenesis, nEPCs were tested on a robust and quantifiable ex-vivo assay using mouse choroidal explants. These explants allow the study of the sprouting and regression of murine vascular endothelial cells under the influence of exogenous angiogenic or anti-angiogenic factors (FIG. 22A and FIG. 22B). This vessel sprouting inhibition assay was performed by exposing mouse choroidal explants to aflibercept and nEPCs respectively at Day 2 of the experiment after initial vessel sprouting (FIG. 24 and FIG. 25). Upon exposure to aflibercept and nEPCs, the sprouting area was reduced from 16.2%±4.0 to 10.0%±0.9 and 1.5%±0.38 respectively. A vessel regression test was performed to exclude the possibility that the results were due to nEPC-induced toxicity and cell death rather than its anti-angiogenic properties. In this test, aflibercept and nEPCs were added only after vessels had been allowed to sprout for 4 days. The area of vessel sprouting was then measured after 48 and 72 hours (FIG. 23). nEPCs-treated explants did not induce a regression of the pre-sprouted vessels (FIG. 26), arguing against toxicity, but reduced further vessel sprouting (FIG. 25) to a comparable extent as observed in aflibercept controls (nEPC: 10.3±2.7% at 48 hr and 11.7±1.5% at 72 hr, aflibercept: 9.9±2.1% at 48 hours and 12.2±1.8% at 72 hours), suggesting an anti-angiogenic effect as opposed to cytotoxicity. These results provide pathological evidence of nEPCs anti-angiogenic effects.

### Example 3: nEPCs Function as Nano-Carriers to Promote Intracellular Uptake of Aflibercept In-Vitro in Human Corneal Epithelium Cells (hCEC)

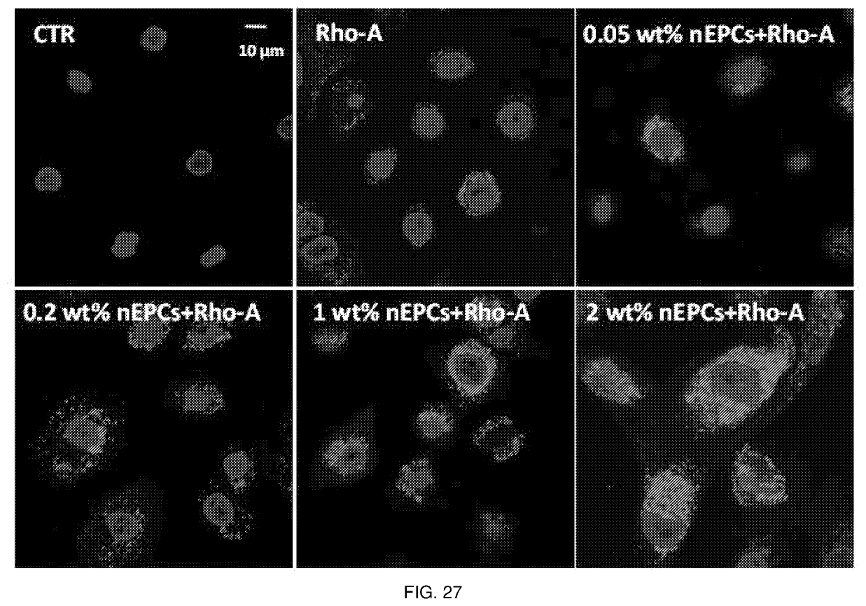

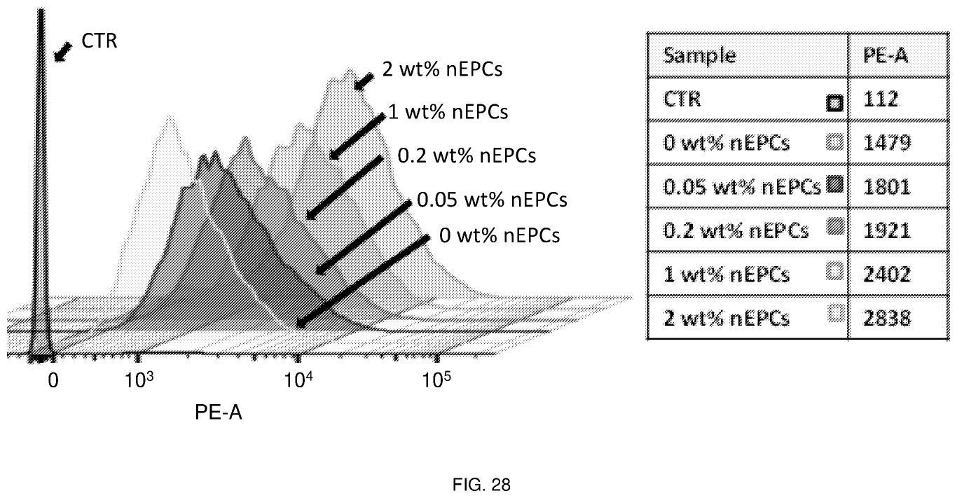

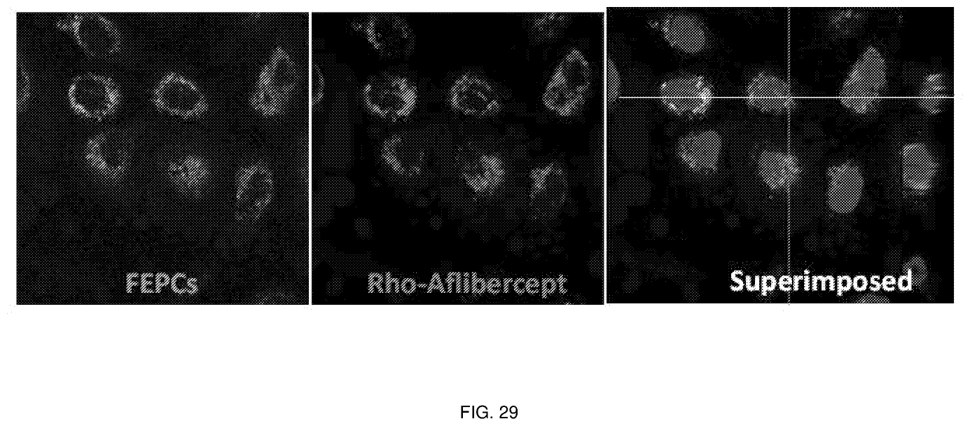

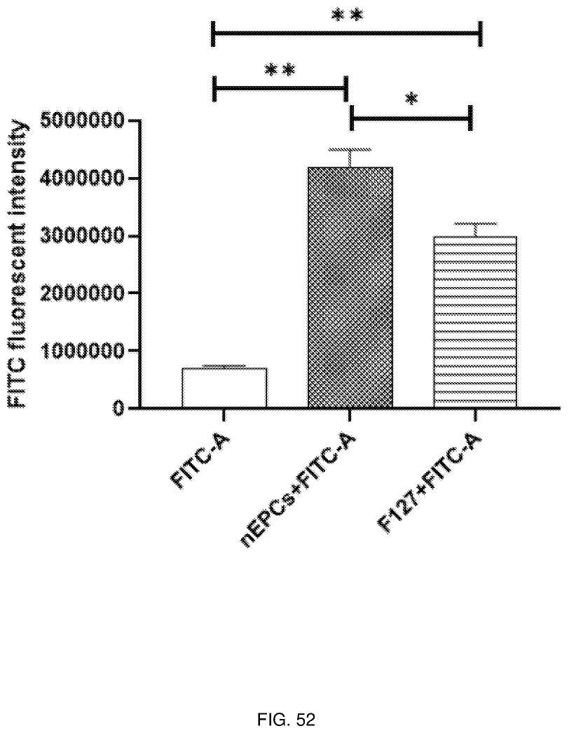

To ascertain if nEPCs are able to function as drug carriers for aflibercept in-vitro, Rho-A was incubated with hCEC for 24 hours. Maximal intracellular uptake of Rho-A was observed at 2 wt % of nEPCs (FIG. 27), suggesting that internalisation of aflibercept increases with increasing nEPC wt %. Flow cytometry was performed to quantify the concentration dependent uptake of Rho-A. Fluorescence intensity emitted by Rho-A was about 2 times higher when 2 wt % nEPCs+Rho-A was administered as compared to just Rho-A alone (FIG. 28). Fluorescence intensity of internalised nEPCs+A was higher compared to F127+Aflibercept (FIG. 52). Confocal Z-stack imaging of hCEC, FEPC and Rho-A also showed co-localisation of FEPC and Rho-A within the cytoplasm of hCEC rather than attachment to the cellular surface, suggesting the intracellular uptake of nEPCs+A (FIG. 29).

### Example 4: nEPCs Enhances Ocular Penetration of Aflibercept in an Ex-Vivo Porcine Model and In-Vivo Murine Model

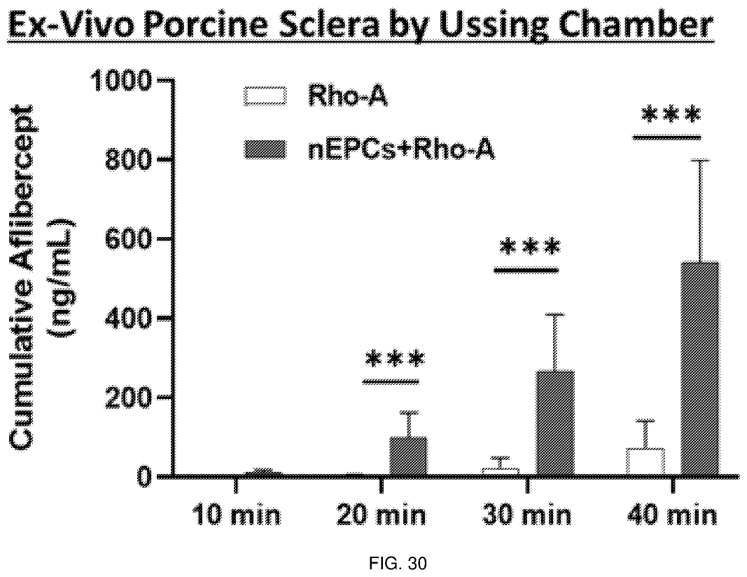

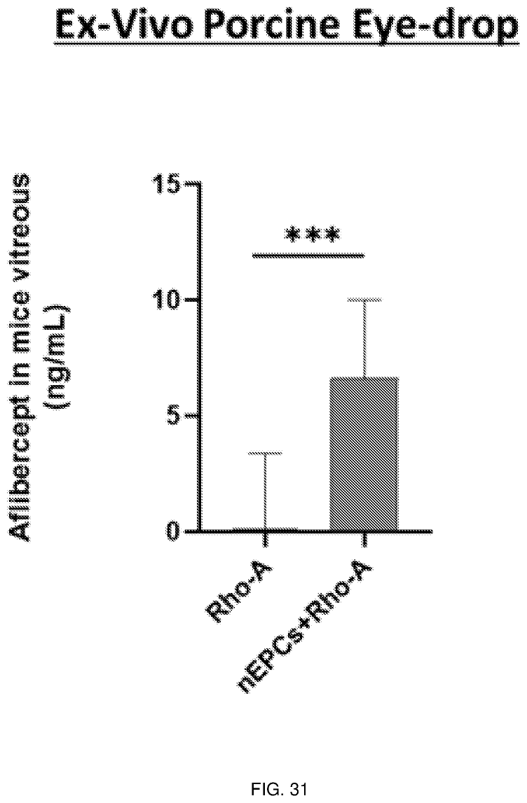

To assess the ability of nEPCs to enhance aflibercept penetration ex-vivo, porcine sclera was excised and clamped on a vertical Ussing chamber, which allowed measurement of drug transport across the tissue. The porcine sclera was exposed to Rho-A or nEPCs+Rho-A continuously for 40 minutes. Throughout the period, the PBS solution at the opposite site was harvested at multiple time points. A higher concentration of Rho-A was detected in the presence of nEPCs+Rho-A (541 ng/mL) compared to just Rho-A alone (70 ng/mL) at 40 min (FIG. 30). Similarly, when tested using a porcine corneal-scleral eyecup, the concentration of Rho-A in the vitreous was 6-fold higher (6 ng/mL) when nEPCs+Rho-A was applied as compared to Rho-A alone (0.09 ng/mL) (FIG. 31).

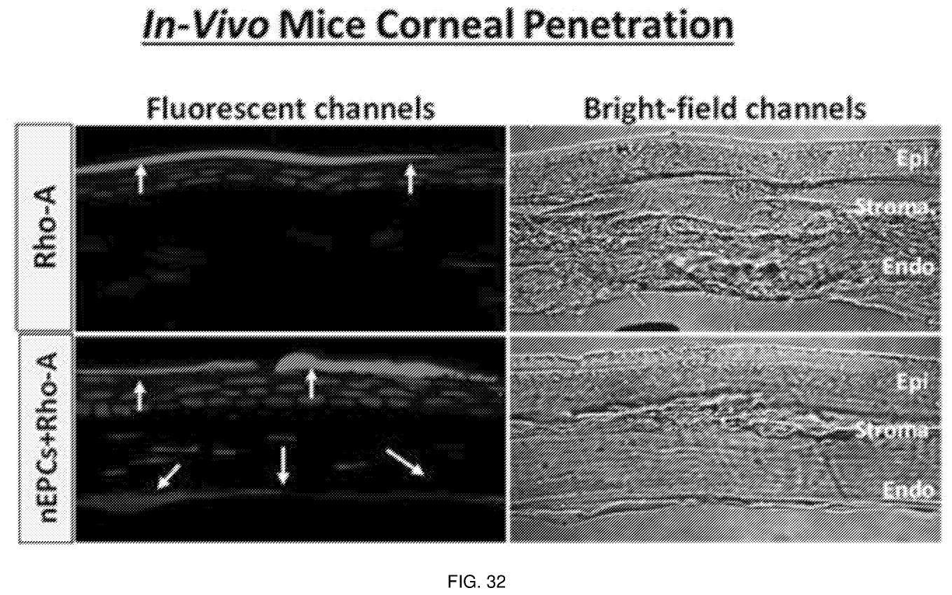

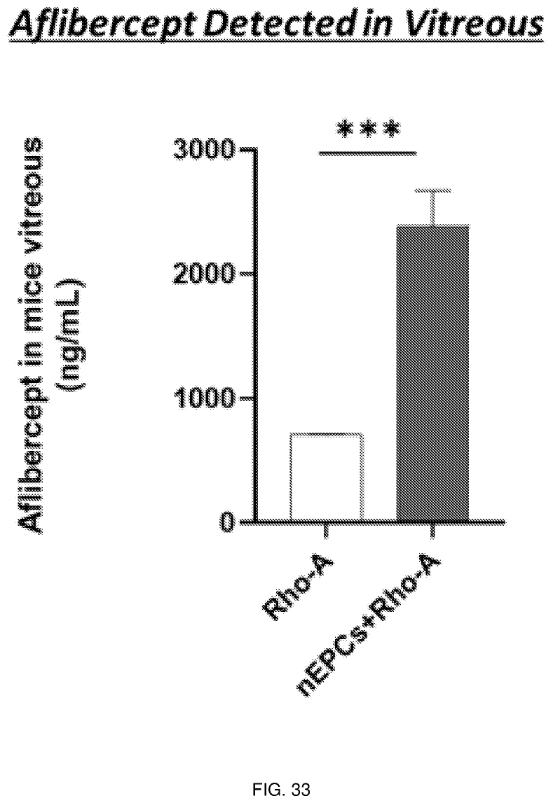

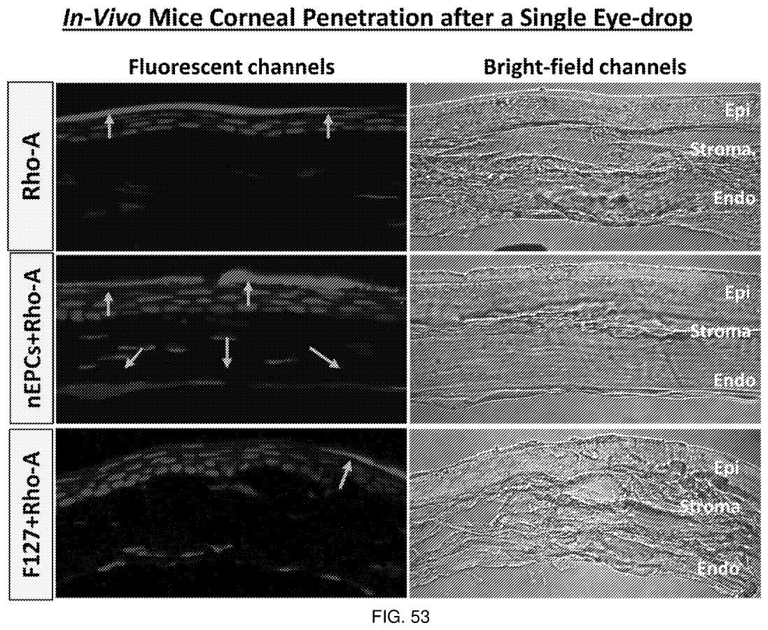

To further validate if nEPCs can facilitate aflibercept corneal penetration in-vivo, wildtype mice (n=3) were treated with a single topical dose of Rho-A (40 mg/mL), nEPCs+Rho-A or F127-Rho-A. It was observed that nEPCs+Rho-A was able to penetrate the cornea, and accumulate beneath the cornea endothelium, while Rho-A alone and F127-Rho-A accumulated above the cornea epithelium (FIG. 32, FIG. 53). Consistent with this, a four-fold higher amount of aflibercept was detected in the vitreous of mice (n=10) treated with nEPCs+Rho-A (2362±354.7 ng/mL) compared to Rho-A alone (633±133.9 ng/mL) (FIG. 33).

### Example 5: nEPCs+Rho-A Prolongs the Corneal Surface Retention Time as Compared to Rho-A Alone

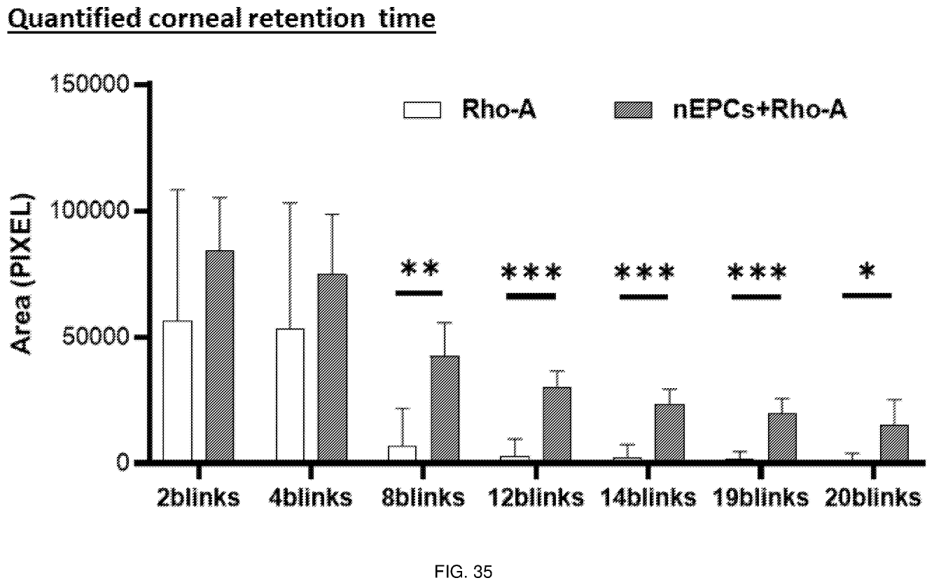

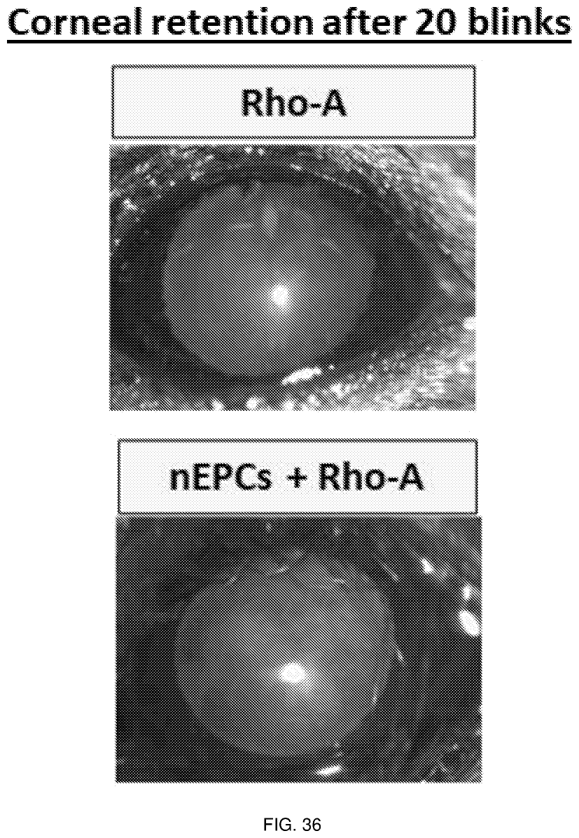

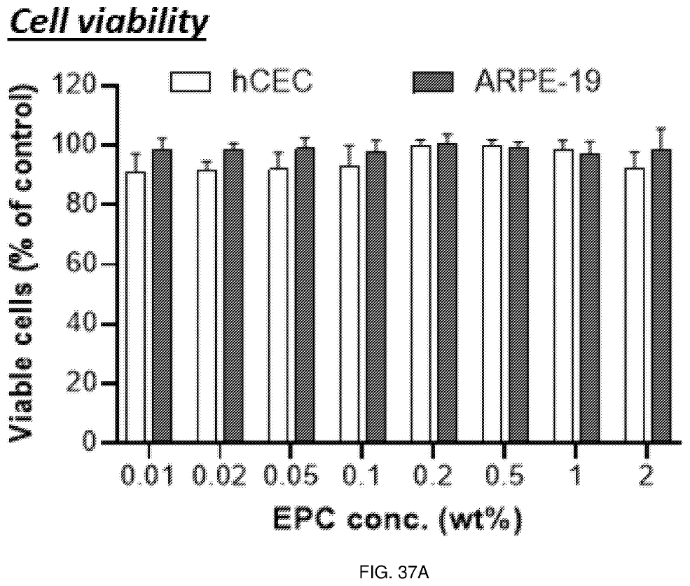

A comparison of corneal retention time of a single drop of nEPCs+Rho-A against Rho-A alone was conducted on mouse eyes (FIG. 34A, FIG. 34B, FIG. 34C, FIG. 35 and FIG. 36). Anterior segment optical coherence tomography (ASOCT) imaging demonstrated the reduction of eyedrop volume on the corneal surface over time and repeated blinks. After 8 blinks at around approximately 120 seconds, there was a significant greater area of nEPCs+Rho-A remaining on the cornea surface (FIG. 35). Anterior segment photos also showed the retention of nEPCs+Rho-A eyedrops on the cornea surface after 20 blinks compared to Rho-A alone (FIG. 36).

### Example 6: nEPCs are Biocompatible with Human Cell Lines In-Vitro and the Murine Eye In-Vivo

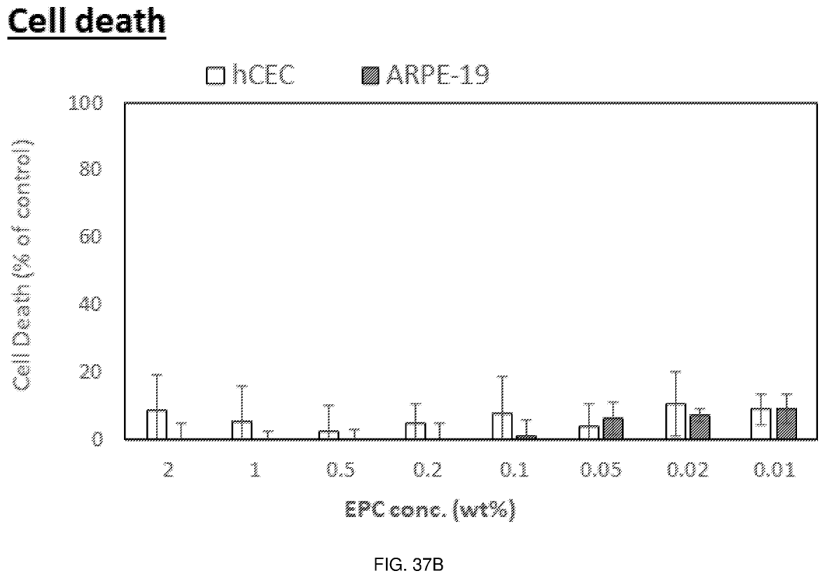

The cytotoxicity of nEPC was evaluated using LDH assay on hCECs and ARPE-19 cells (a human retinal pigment epithelial cell line) (FIG. 37A). Minimal cell death was observed after 24 hours of co-culture with EPC polymer of concentrations ranging from 0.01-2 wt % (FIG. 37B).

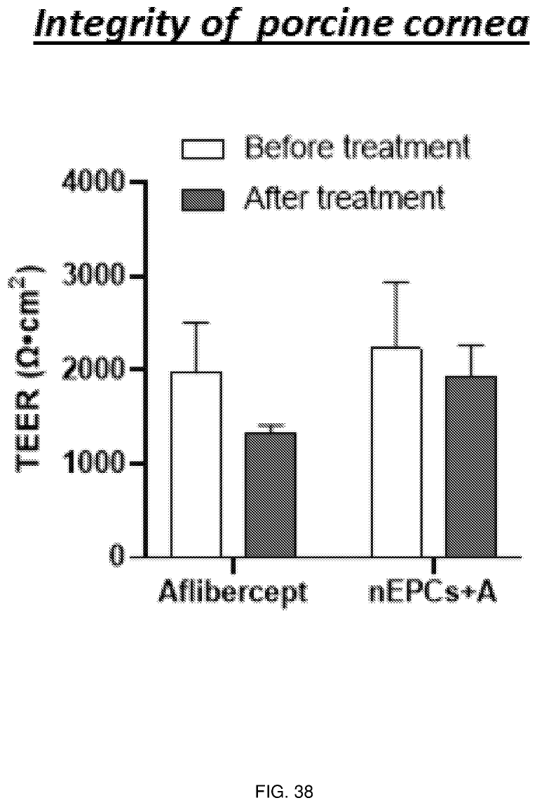

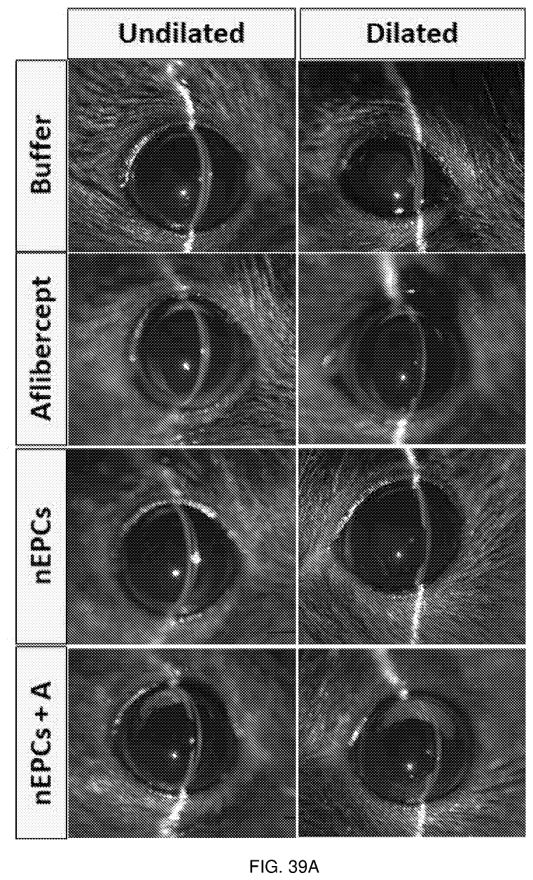

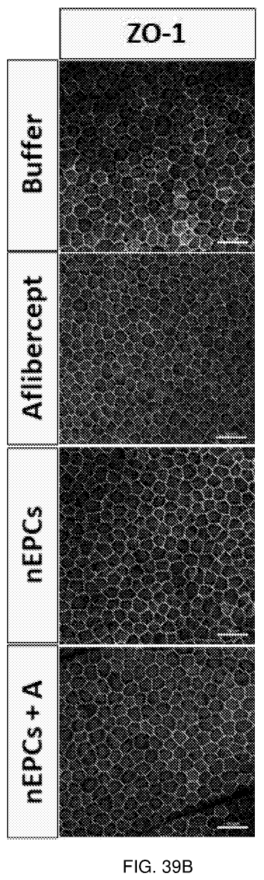

To assess nEPCs effect on the corneal barrier integrity, the transepithelial electrical resistance (TEER) was measured for porcine cornea after prolonged exposure to aflibercept and nEPCs+A respectively. TEER are strong indicators of cellular barrier integrity. When comparing before and after exposure, no significant change in TEER was measured for both aflibercept and nEPCs+A exposure, suggesting no disruption to the corneal barrier after exposure to nEPCs+A (FIG. 38).

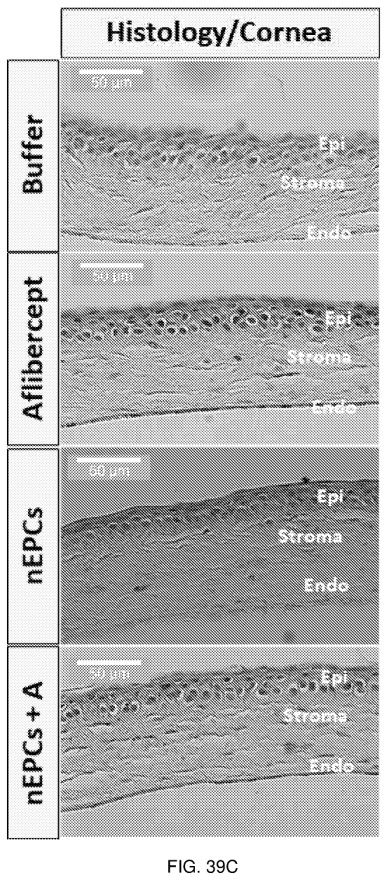

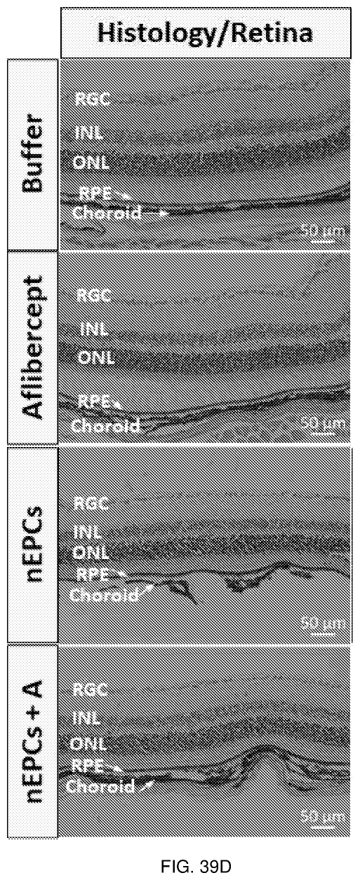

To evaluate the biocompatibility of topically applied nEPCs, wildtype mice eyes (n=8) were treated with either buffer, aflibercept, nEPCs or nEPCs+A solutions. For each eye, the solutions were administered 3 times a day for a total duration of 14 days. Anterior segment images using the slit-lamp microscope were taken of undilated and dilated eyes to assess cornea and lens clarity. At Day 14, both the nEPCs and nEPCs+A treated eyes did not demonstrate any cornea or lens opacities when compared to the eyes treated with the control buffer solution. Histology sections of the cornea demonstrated an intact corneal histology with the absence of infiltration by inflammatory cells in both the nEPCs and nEPCs+A treated eyes. ZO-1 immunofluorescence staining, a marker for tight junctions in the cornea epithelial layer, demonstrated maintenance of corneal tight junctions in the nEPCs and nEPCs+A treated eyes. Terminal deoxynucleotidyl transferase dUTP nick end labelling (TUNEL) staining did not demonstrate increased apoptosis in both the nEPCs and nEPCs+A treated eyes. This was similar to the buffer control group. Furthermore, all corneal cells exhibited large polygonal, squamous cell shapes and clear cell boundaries. These findings suggest that the nEPCs and nEPCs+A did not have an overt adverse effect on both the corneal epithelial cells and the retina cells, and are thus biocompatible (FIG. 39A, FIG. 39B, FIG. 39C, FIG. 39D and FIG. 39E).

### Example 7: Topically Applied nEPCs+a is Able to Reduce Vessel Leakage in a Laser-Induced CNV Mice Model

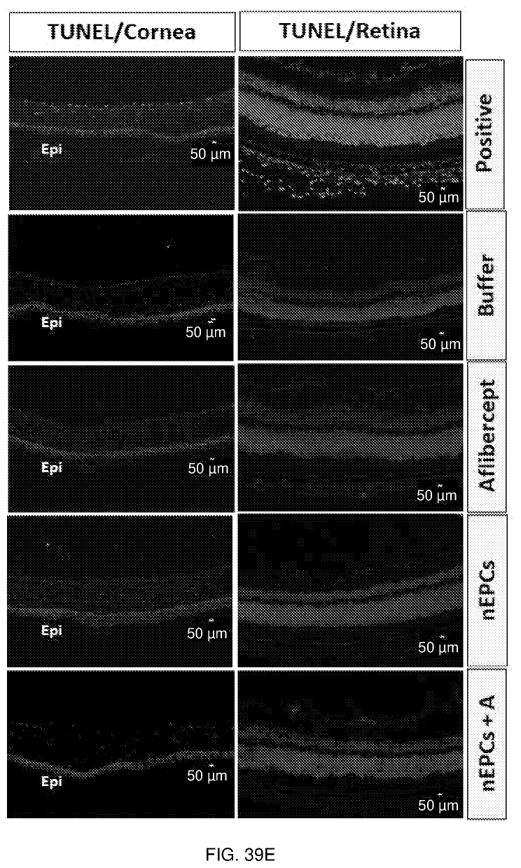

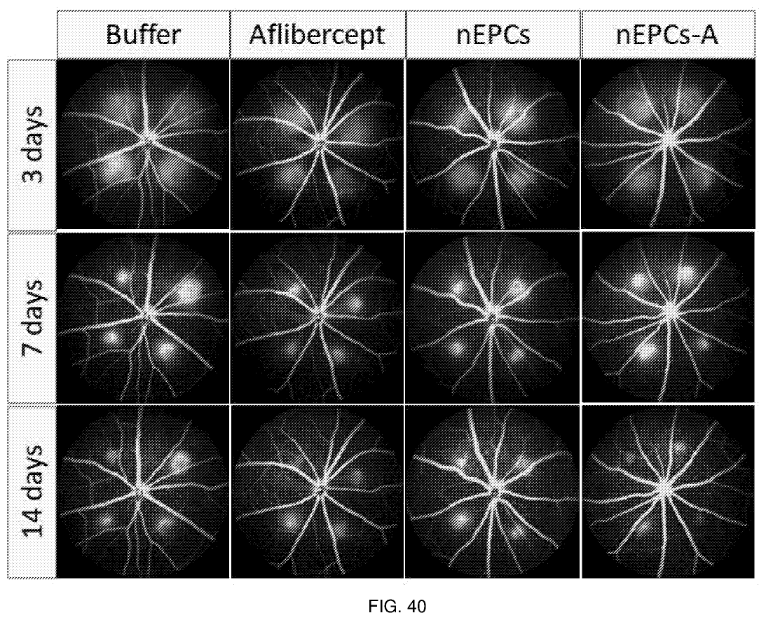

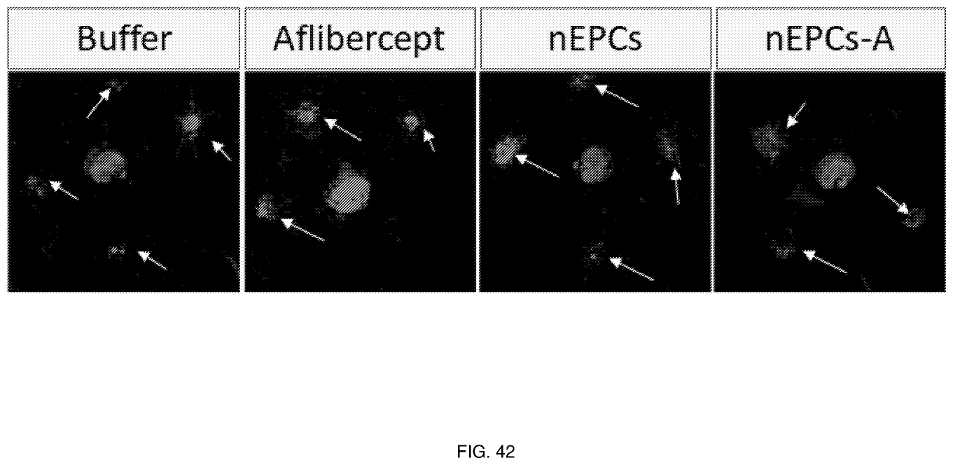

The bioactivity of nEPCs+A was determined using a mouse model of laser-induced CNV. CNV eyes were treated three times daily for 14 days with either buffer, aflibercept (40 mg/mL), nEPCs or nEPCs+A solution. Fundus fluorescein angiography (FFA) was performed on day 3, 7 and 14 respectively, to monitor the resolution of vascular leakage in response to treatment (FIG. 40) and the recovery rate was calculated (see Experimental Methods in Example 10 below) (FIG. 41). While the area of leakage was not reduced when buffer solution was applied, recovery rate of 118.3 48.7; 239.7±108.8; 568.1±68.9 pixels/day were observed for eyes applied with aflibercept, nEPCs and nEPCs+A, respectively. A significant faster recovery was found for nEPC+A as compared to buffer. At the end of 14 days, eyes were enucleated to obtain choroidal flat mounts for isolectin B4 staining of endothelial cells (FIG. 42). Choroidal flatmounts allow a panoramic view of the CNV lesions within the choroidal tissue, providing pathological evidence for comparison of CNV lesion sizes. Consistent with the FFA results, eyes treated with nEPC+A had the smallest area of CNV lesion remaining.

### Example 8: Discussion

The IVT of anti-VEGF compounds remain the mainstay of treatment for retinal vascular diseases. However, due to the treatment burden and potential sight-threatening complications associated with IVTs, the topical delivery of anti-VEGF compounds via eyedrops represents a much more desired and accessible mode of repeated anti-VEGF delivery to the retina. Ideally, a topical anti-VEGF delivery system must be able to 1) overcome ocular barriers to deliver a therapeutic concentration of drug into the posterior segment of the eye, 2) demonstrate biocompatibility, particularly for repeated use and 3) preserve bioactivity as well as therapeutic effect at the retina. To date, published approaches have only demonstrated limited success in fulfilling these criteria. In particular, it has been challenging to achieve a therapeutic concentration of anti-VEGF in the posterior segment of the eye for disease control. In the preceding examples, the results have shown that nEPCs+A is capable of overcoming these hurdles that have been halting the successful development of an effective topical anti-VEGF formulation.

In comparison to other nanoformulations, it is understood that nEPCs is better able to fulfil the above criteria of an ideal topical anti-VEGF delivery system. Importantly, nEPCs+A was capable of achieving a therapeutic concentration of aflibercept in the posterior segment of the murine eye, as assessed in a validated disease model. When a single drop of nEPCs+A was administered on the murine eye, a four-fold higher amount of aflibercept was detected in the vitreous of mice treated with nEPCs+A compared to topical aflibercept alone. nEPCs+A could achieve an aflibercept concentration of up to 2362.5 ng/mL±354.6 in the vitreous. This is higher than the IC₅₀ of aflibercept for VEGF which has been reported to be approximately 1.8 ng/ml. Consistent with the mouse model, when administered topically on the ex-vivo porcine eye model, nEPCs+A could achieve an aflibercept concentration of up to 6 ng/mL in the vitreous as compared to aflibercept alone. These results suggest that nEPCs+A was able to significantly enhance the delivery of topically administered aflibercept to the posterior segment of the eye. More importantly, nEPCs+A could achieve an aflibercept concentration in the murine vitreous which was above the clinically significant concentration required to inhibit VEGF activity.

Without being bound by theory, it is believed that the ability of nEPCs+A to achieve a therapeutic concentration of aflibercept in the retina may be due to two reasons. Firstly, nEPCs+A has a high EE. In the experiments, nEPCs+A was capable of achieving a 47.3% aflibercept EE. Prior work on topical delivery of approved anti-VEGF compounds to the retina utilised liposomes to entrap bevacizumab. These liposomes contained AnxA5 which enhanced the uptake of the liposomal drug carrier across corneal epithelial barriers. In comparison, the AnxA5-associated liposomes had an EE of between 22-25%, which is nearly half of the EE achieved in nEPCs+A. As nEPCs+A has a significantly greater EE, it is able to package and eventually deliver a larger drug payload to the posterior segment of the eye. Secondly, nEPCs+A was capable of enhancing the delivery of aflibercept across corneal epithelial and scleral barriers. This was demonstrated by both an ex-vivo porcine cornea model using a Ussing chamber and when tested in the in-vivo mouse model, whereby nEPCs+A was able to penetrate the cornea to reach the endothelial layer. The barrier function of the cornea was not disrupted significantly as shown by the preservation of TEER. While the exact mechanism by which nEPCs facilitates cornea penetration remains to be further investigated, nEPCs+A were noted to be taken up by corneal epithelial cells in-vitro, suggesting the possible movement of nEPCs+A through the cornea via transcytosis. The cornea surface retention experiments also suggest that nEPCs+A remained on the cornea surface longer as compared to aflibercept solution. A longer retention time may result in better uptake of the nanomicelles by the corneal epithelial cells. Altogether, these results suggest that nEPCs+A is capable of acting as a carrier to facilitate the corneal penetration of aflibercept for posterior segment drug delivery without disrupting the function or structure of the corneal barrier. The subsequent route which nEPCs+A take after cornea penetration can be inferred from the performed experiments. The significantly greater accumulation of aflibercept in the vitreous of the porcine ex-vivo model after a single eyedrop of nEPCs+RhoA and the reduction of CNV lesions after administration of nEPCs+A eyedrop suggest that nEPCs+A can overcome the vitreous to reach the retina for its intended activity.

To enable safe drug delivery from the ocular surface to the posterior segment of the eye, the demonstration of nEPC's biocompatibility was crucial. When co-cultured with both cornea (hCEC) and retinal pigment epithelium (ARPE-19) cell lines, nEPCs+A demonstrated good biocompatibility. This was further proven in in-vivo mice models, when nEPCs+A were repeatedly administered on the ocular surface over a period of 14 days, the cornea remained clear. Histological analysis demonstrated no change in the morphology and organisation of both corneal epithelial and endothelial cells. In particular, ZO-1, a marker of the tight junction in the corneal epithelium, was not disrupted as compared to control experiments. Tight junctions are extremely crucial to cornea homeostasis as they constitute the principal barrier to passive movement of fluid, electrolytes, macromolecules and cells. Given that nEPCs+A may move through the cornea via transcytosis, these results suggest that transcytosis did not cause any toxicity to the cornea in the short-term. Administration of nEPCs+A also did not result in accelerated cataract formation. The results also suggest that nEPCs+A were able to reach the posterior segment of the eye without affecting the structure or function of both the cornea and lens. This is particularly important as the cornea and lens are the main refractive components of the eye. Hence, any inflammation in these tissues may reduce the eventual visual acuity.

nEPCs+A also managed to retain the bioactivity of aflibercept in the posterior segment. This was suggested by the results from the administration of nEPCs+A on in-vivo mouse laser-induced CNV models. After 2 weeks of consecutive treatment, nEPCs+A treated eyes had the greatest rate of CNV regression as compared to aflibercept or nEPCs alone. This suggests a synergistic effect between the anti-angiogenic effects of both aflibercept compound and nEPCs.

The observation that nEPCs alone can inhibit angiogenic activity in-vitro and ex-vivo is unexpected. In the HUVEC migration, proliferation and tube formation studies, it was noted that nEPCs could also inhibit the VEGF-driven processes. The effect of nEPCs alone was sometimes greater than the effect of aflibercept alone, was observed both in-vitro and ex-vivo. RNA expression analysis of HUVECs treated with nEPCs also suggests that the anti-angiogenic effects could be mediated by both VEGF and non-VEGF mediated pathways. Furthermore, nEPCs and aflibercept downregulate separate angiogenic pathways. Aflibercept mainly downregulates VEGFR1 while nEPC downregulates both VEGF-C and VEGFR3 pathways, suggesting a possible two-pronged mechanism that can contribute to better anti-angiogenic effects. To further understand the effect of nEPCs on the process of angiogenesis, a study using an AIM chip was performed. This allowed the study of angiogenesis in a 3D micronetwork, whereby cell-cell and cell-extracellular matrix interaction can be studied simultaneously. Specifically, the use of a differential VEGF gradient was used to induce HUVECs sprouting into the middle collagen-filled channel. The observations from this 3D vascular micronetwork experiment shed greater light on the anti-angiogenic mechanisms of nEPCs. While cellular proliferation and tube formation continued to be inhibited, HUVEC migration was not significantly inhibited. Without being bound by theory, it is believed that the phenomenon could be due to the addition of collagen. It is known that immobilised extracellular matrix components such as collagen drive endothelial cell migration independently of chemotactic cytokines—known as haptotaxis. It is therefore postulated that nEPCs were able to inhibit VEGF-driven angiogenesis pathways responsible for endothelial cellular proliferation and tube formation but not haptotaxis which is driven by ECM components such as collagen.

While the pharmacokinetics of a smaller murine eye are not identical to the human eye well due to the disparity in size, this is partially addressed by using an ex-vivo porcine model, whereby similar results were yielded and nEPCs+A were able to achieve a larger amount of corneal penetration. The present disclosure may also serve as a platform for further studies to be carried out to further explore the mechanisms behind the anti-angiogenic properties of nEPCs, as angiogenesis is a dynamic process regulated by various proangiogenic mediators and anti-angiogenic factors to enable endothelial cell proliferation, migration, adhesion and tube formation. For example, proteomic analysis may be utilised to look for modulation of angiogenesis signalling pathways by nEPCs. Furthermore, additional studies into other possible routes of delivery such as trans-scleral pathways may be conducted to elucidate the full mechanism of nEPCs+A.

### Example 9: Summary

In summary, this study discusses a novel topical formulation consisting of aflibercept, an anti-VEGF compound, encapsulated by a polymeric nanomicelle with intrinsic anti-angiogenic properties. Apart from being a drug carrier with a high payload and cornea barrier penetration enhancer, the results of this study also suggest the intrinsic anti-angiogenic properties of nEPCs, which may augment the antiangiogenic effect of aflibercept. It is understood that this is the first report of topically administered polymeric micelles loaded with macromolecular biologics and showing therapeutic effect at the retina, and also the first study reporting intrinsic anti-angiogenic effects of nEPCs. nEPC is capable of fulfilling the necessary characteristics for an effective topical anti-VEGF delivery system for retinal diseases. Together, the ability to deliver a therapeutically significant concentration of aflibercept to the retina and the intrinsic anti-angiogenic properties of nEPCs, suggest synergistic effects which can be harnessed for the effective topical delivery of existing anti-VEGF compounds. This suggests that nEPCs+A may be a promising topical drug formulation for the treatment of retinal diseases.

### Example 10: Experimental Methods

### 10.1. Materials and Reagents

PEG, PPG and PCL were obtained from Sigma-Aldrich (Missouri, United States). Pluroic F127 (P2443) was purchased form Sigma. Aflibercept was obtained from Bayer Healthcare (Berlin, Germany). NHS-Fluorescein and NHS-Rhodamine were obtained from Thermo Fisher Scientific (Waltham, MA USA).

The 3D Cell Culture Chips were obtained from AIM BIOTECH (Singapore). Lactate Dehydrogenase Release (LDH) assay kit was obtained from DojinDo EU (Kumamoto, Japan). Optimal cutting temperature (OCT) compound (Tissue-Tek®) was obtained from Sakura Finetek (USA).

A chemical structure of an exemplary copolymer designed in accordance with various embodiments disclosed herein is shown in Scheme 1. The polymer is a tri-component multi-block thermogelling polymer which consists of hydrophilic poly(ethylene glycol) (PEG), thermosensitive poly(propylene glycol) (PPG), and hydrophobic biodegradable polyesters such as, but not limited to, biodegradable poly(s-caprolactone) (PCL) segments linked together via urethane bonds.

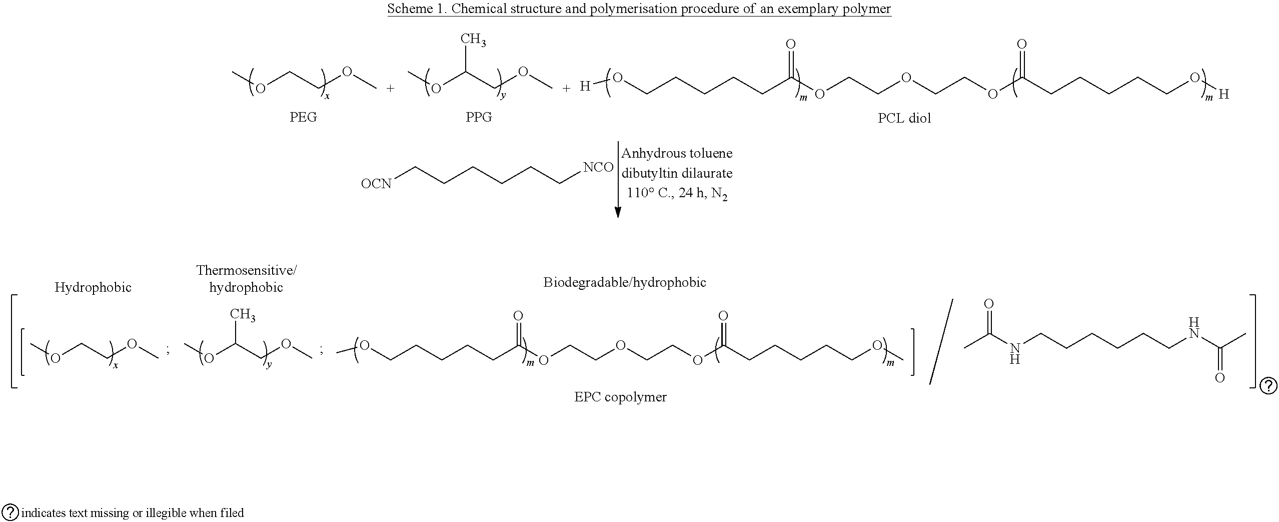

### Synthesis of Polymer

The general steps for preparing a multi-block copolymer in accordance with various embodiments disclosed herein include: mixing one or more hydrophilic polymers, one or more hydrophobic polymers and one or more thermosensitive polymers with a coupling agent (in the example below, 1,6-diisocyanatohexane was used) in the presence of a metal catalyst (in the example below, dibutyltin dilaurate was used) and a suitable solvent (in the example below, toluene was used), as shown in Scheme 1.

An example of preparing a polymer designed in accordance with various embodiments disclosed herein is described in detail as follows.

Poly(PEG/PPG/PCL urethane) was synthesized from PEG, PPG, and PCL-diol using 1,6-Diisocyanatohexane as a coupling reagent. The amount of 1,6-Diisocyanatohexane added was equivalent to the reactive hydroxyl groups in the solution. Typically, 0.15 g of PCL-diol (Mn=2000, 7.50×10⁻⁵ mol), 12 g of PEG (Mn=2050, 5.85×10⁻³ mol), and 3 g of PPG (Mn=2000, 1.50×10⁻³ mol) were dried in a 250-mL two-neck flask at 50° C. under high vacuum overnight. Then, 100 mL of anhydrous 1,2-toluene was added to the flask, and any trace of water in the system was removed through azeotropic distillation performed twice. 100 mL of anhydrous 1,2-toluene was added to the flask, then two drops of dibutyltin dilaurate (˜8×10⁻³ g) and 1.27 g of 1,6-Diisocyanatohexane (7.58×10⁻³ mol) were added sequentially. The reaction mixture was stirred at 60-110° C. under a nitrogen atmosphere for 24 h. The resultant copolymer was precipitated from diethyl ether and further purified by re-dissolving into chloroform, followed by precipitation in diethyl ether. The yield was 85% after isolation and purification.

### 10.2. Preparation of EPC Co-Polymer and Fluorescein-Containing EPC Polymer (FEPC)

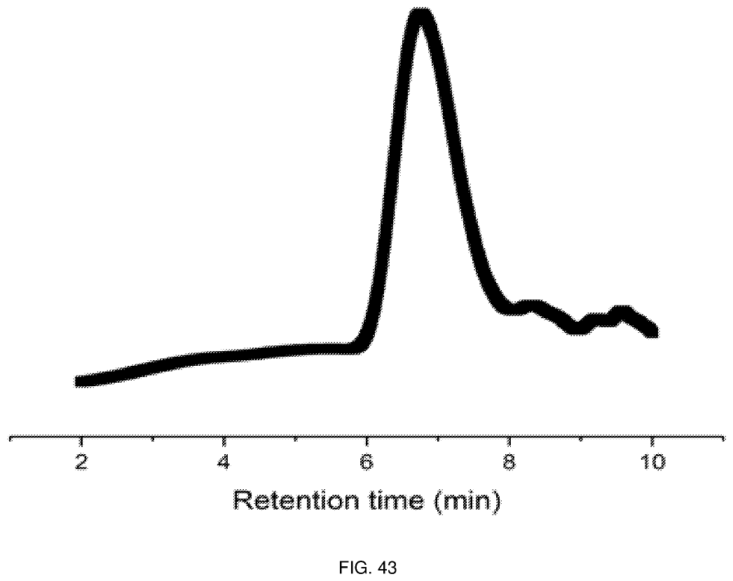

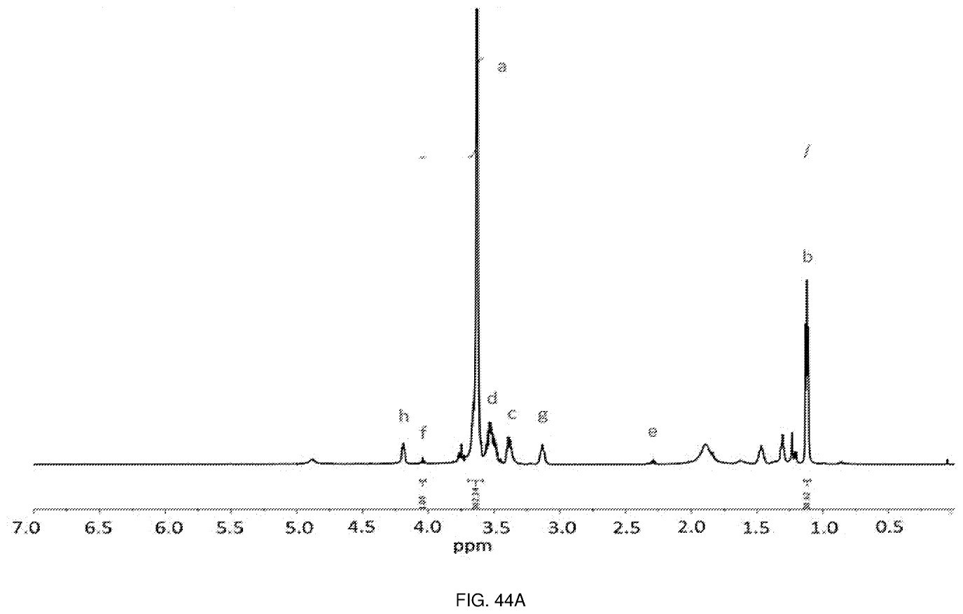

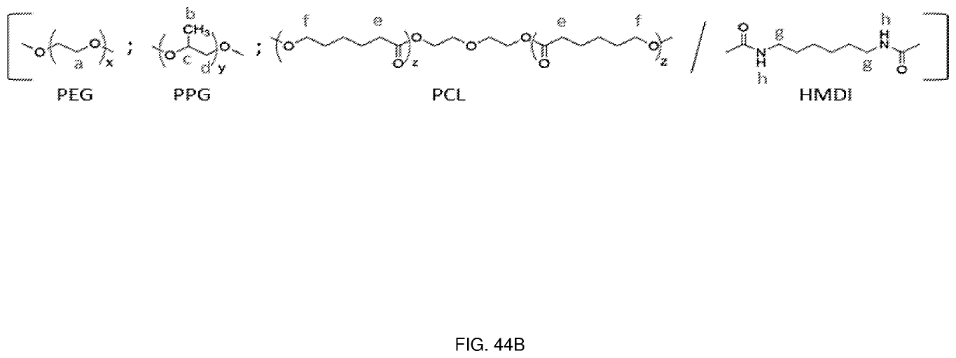

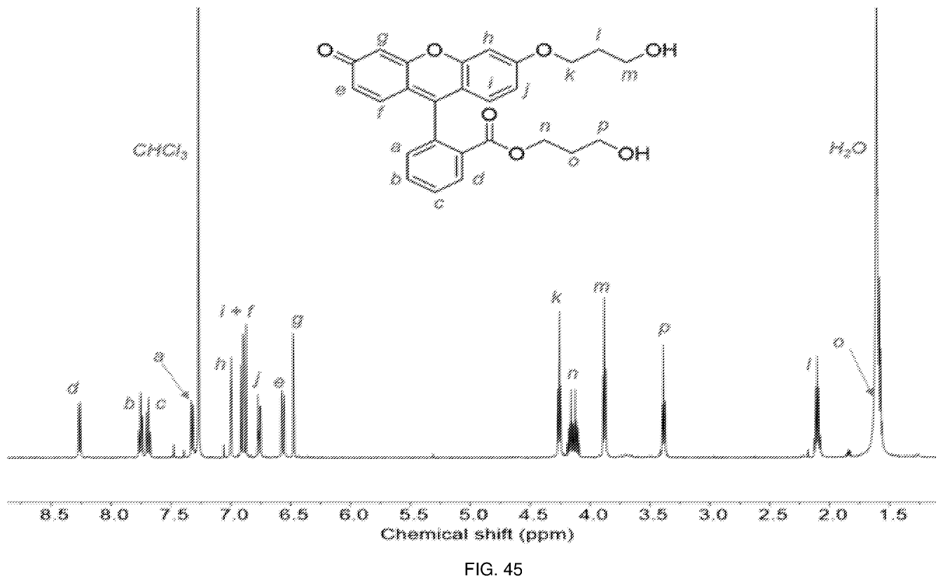

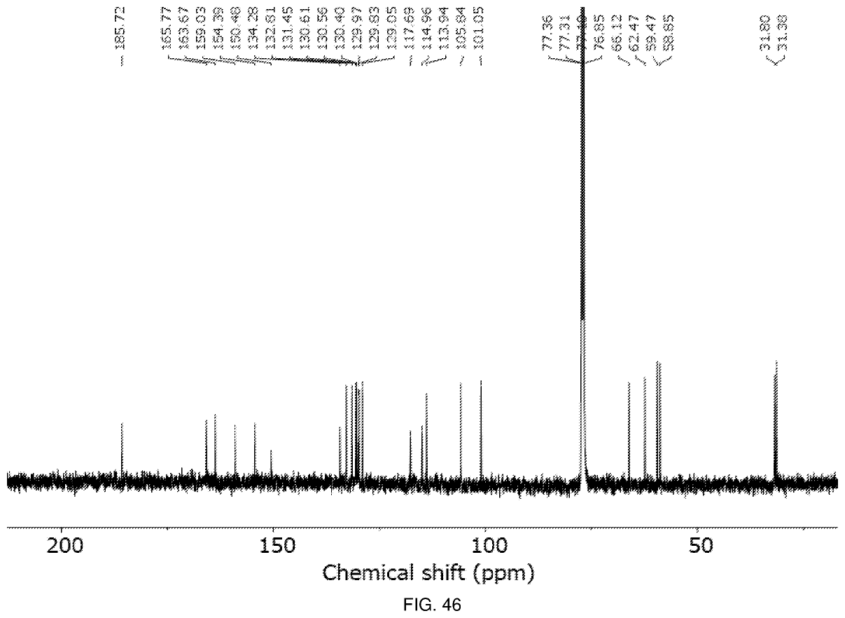

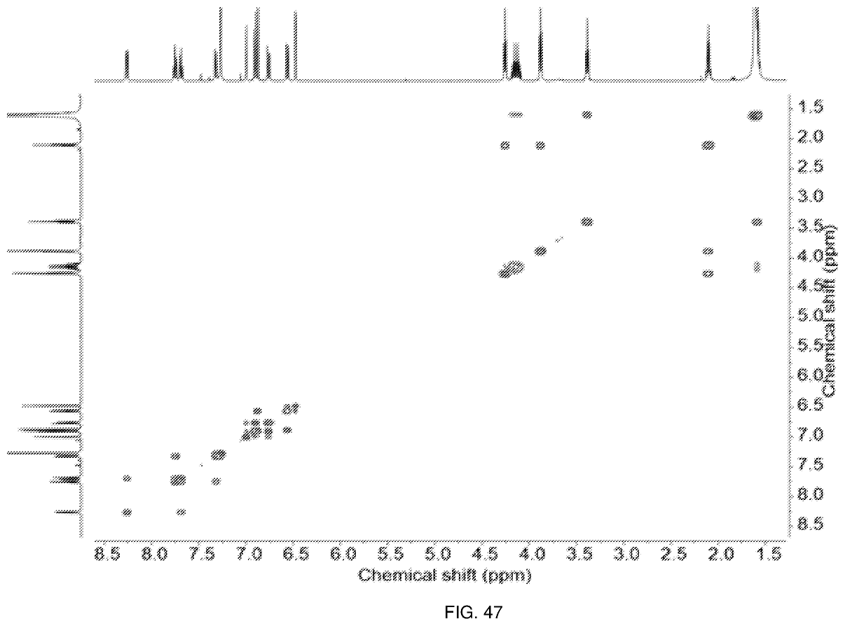

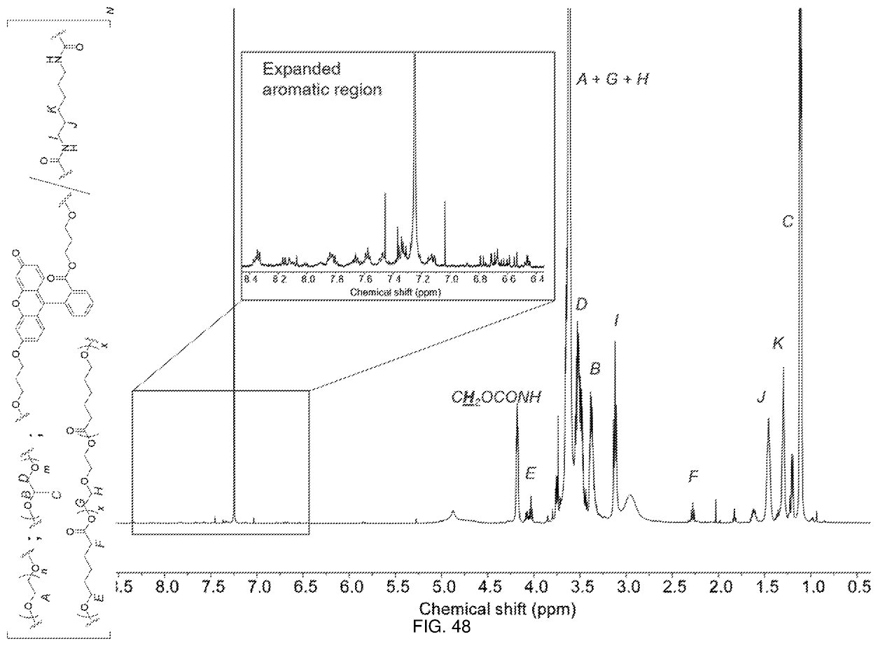

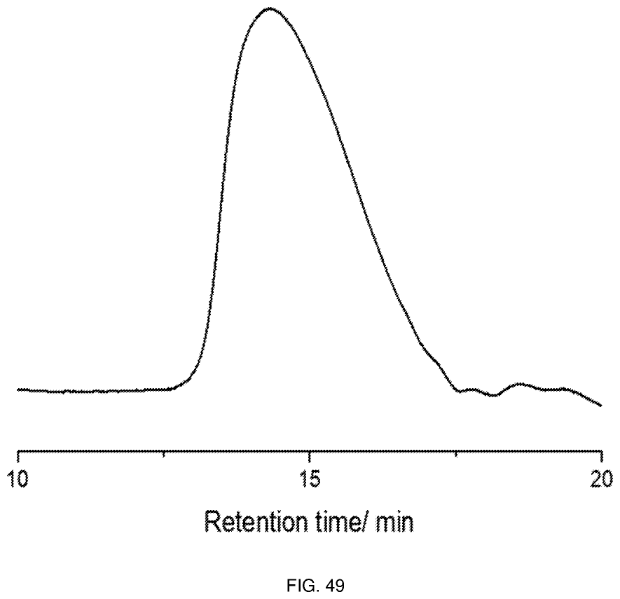

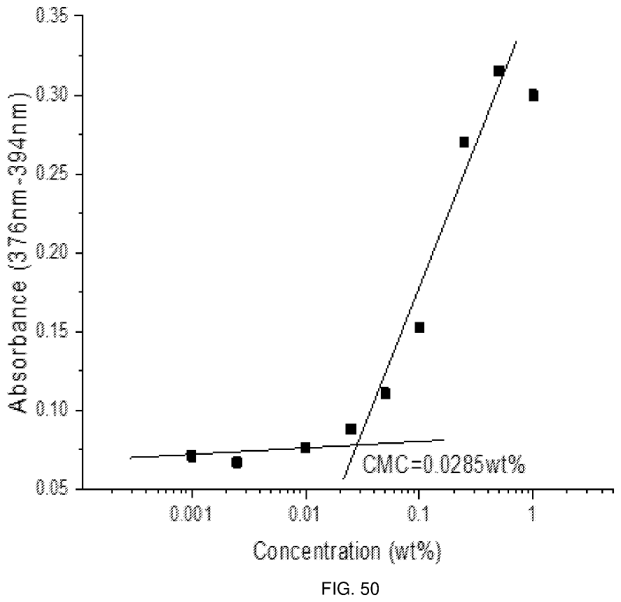

EPC co-polymer was synthesized by linking PEG, PPG and PCL. The feed ratio of PEG:PPG was fixed at 4:1, together with PCL (1%). To make FEPC, PEG (4.0 g, average MW 2050), PPG (1.0 g, average MW 2000) and PCL (50 mg, average MW 2000) were dried by azeotropic distillation using anhydrous toluene (2×20 mL) on a rotary evaporator, followed by heating at 110° C. for 1 hour in vacuo. Thereafter, fluorescein-diol (75 mg) (Scheme 2) was added portionwise to the mixture, followed by the zinc diethyldithiocarbamate (12.4 mg) catalyst, anhydrous toluene (30 mL) and hexamethylene diisocyanate (0.44 mL). The reaction was stirred at 300 RPM for 2 hours at 110° C. The bright yellow polymer was isolated by precipitating the hot toluene solution in vigorously-stirred diethyl ether (500 mL). The resulting polymer was purified by dialysis for 3 days in distilled water using dialysis tubings (MWCO 3500 Da), followed by lyophilisation to give a yellow solid (yield=4.5 g, 81%). FIG. 43 shows the retention time of EPC copolymer in gel permeation chromatography (GPC) and Table 1 shows molecular weight details of the EPC copolymer. Molecular weight of constituent polymers (Table 2), EPC (FIG. 44 & Table 2) fluorescein-diol (FIG. 45 to FIG. 47) and FEPC (FIG. 48 to FIG. 50) were determined by gel permeation chromatography (GPC) and chemical composition was assessed using ¹H nuclear magnetic resonance (NMR). CMC values of FEPC were determined using a dye solubilisation method (FIG. 48 to FIG. 50).

Scheme 2 shows the synthesis methods of (A) Fluorescein-diol from fluorescein and (B) fluorescein-EPC polyurethane random block-copolymer by polyaddition reactions between diol reagents and hexamethylene diisocyanate, catalysed by zinc diethyldithiocarbamate. PEG with average Mₙ of 2050 g mol⁻¹, PPG with average Mₙ of 2000 g mol⁻¹, PCL with average Mₙ of 2000 g mol⁻¹, 1,6-hexamethylene diisocyanate (HMDI, 99%), dibutyltin dilaurate (DBTL, 95%), zinc diethyldithiocarmate (ZDTC, 97%), potassium carbonate, potassium iodide, 3-bromo-1-propanol were purchased from Sigma-Aldrich. Anhydrous toluene was purchased from Tedia, N,N-dimethylformamide (DMF) was purchased from Sigma Aldrich, ethyl acetate was purchased from VWR Chemicals, dichloromethane from Fisher Scientific, methanol (CMOS grade) from J. T. Baker. All chemicals, reagents and solvents were used as received without further purification. Fluorescein (600 mg, 1.81 mmol) was dissolved in anhydrous DMF (6 mL) under sonication and mild heating. To the bright red solution was added potassium carbonate (525 mg, 3.80 mmol), potassium iodide (60 mg, 0.36 mmol) and 3-bromo-1-propanol (0.34 mL, 3.80 mmol). The reaction was heated at 80° C. overnight with vigorous stirring under an Ar atmosphere. Thereafter, the crude reaction was poured into water (100 mL) to form a bright orange suspension, which was extracted with ethyl acetate (5×30 mL). The combined organics were washed with brine (2×30 mL), dried with MgSO₄, and the solvent removed under reduced pressure on a rotary evaporator. Column chromatography (eluent: 10 v/v % methanol in dichloromethane) afforded the target product as a bright orange powder (yield 340 mg, 42%). ¹H NMR (500 MHz, Chloroform-d) b 8.26 (d, J=7.6 Hz, 1H, H_{d}), 7.75 (td, J=7.5, 1.4 Hz, 1H, H_{b}), 7.69 (td, J=7.7, 1.4 Hz, 1H, H_{c}), 7.32 (d, J=7.6 Hz, 1H, Hₐ), 7.00 (d, J=2.4 Hz, 1H, Hₕ), 6.91 (d, J=9.0 Hz, 2H, Hᵢ), 6.88 (d, J=9.6 Hz, 1H, H_{f}), 6.76 (dd, J=9.0, 2.4 Hz, 1H, Hⱼ), 6.56 (dd, J=9.6, 2.0 Hz, 1H, Hₑ), 6.48 (dd, J=1.9, 0.5 Hz, 1H, H_{g}), 4.26 (t, J=6.1 Hz, 2H, Hₖ), 4.21-4.07 (m, 2H, Hₙ), 3.88 (t, J=6.2 Hz, 2H, Hₘ), 3.39 (td, J=6.2, 1.2 Hz, 2H, Hₚ), 2.10 (quint., J=6.0 Hz, 2H, Hᵢ), 1.59 (quint., J=6.0 Hz, 2H, Hₒ). ¹³C NMR (125 MHz, Chloroform-d) δ 185.5, 165.9, 163.7, 159.1, 154.5, 150.5, 134.3, 132.9, 131.5, 130.7, 130.6, 130.5, 130.0, 129.9, 129.1, 117.7, 115.0, 114.0, 105.9, 101.1, 66.1, 62.6, 59.6, 58.9, 31.9, 31.4. λₘₐₓ (DMSO)/nm 437 (ε/dm³ mol⁻¹ cm⁻¹ 39000), 460 (47100), 488 (30500). MS (ESI+ve) m/z 449.093 ([M+H]⁺, C₂₆H₂₄O₇, calc. 449.160).

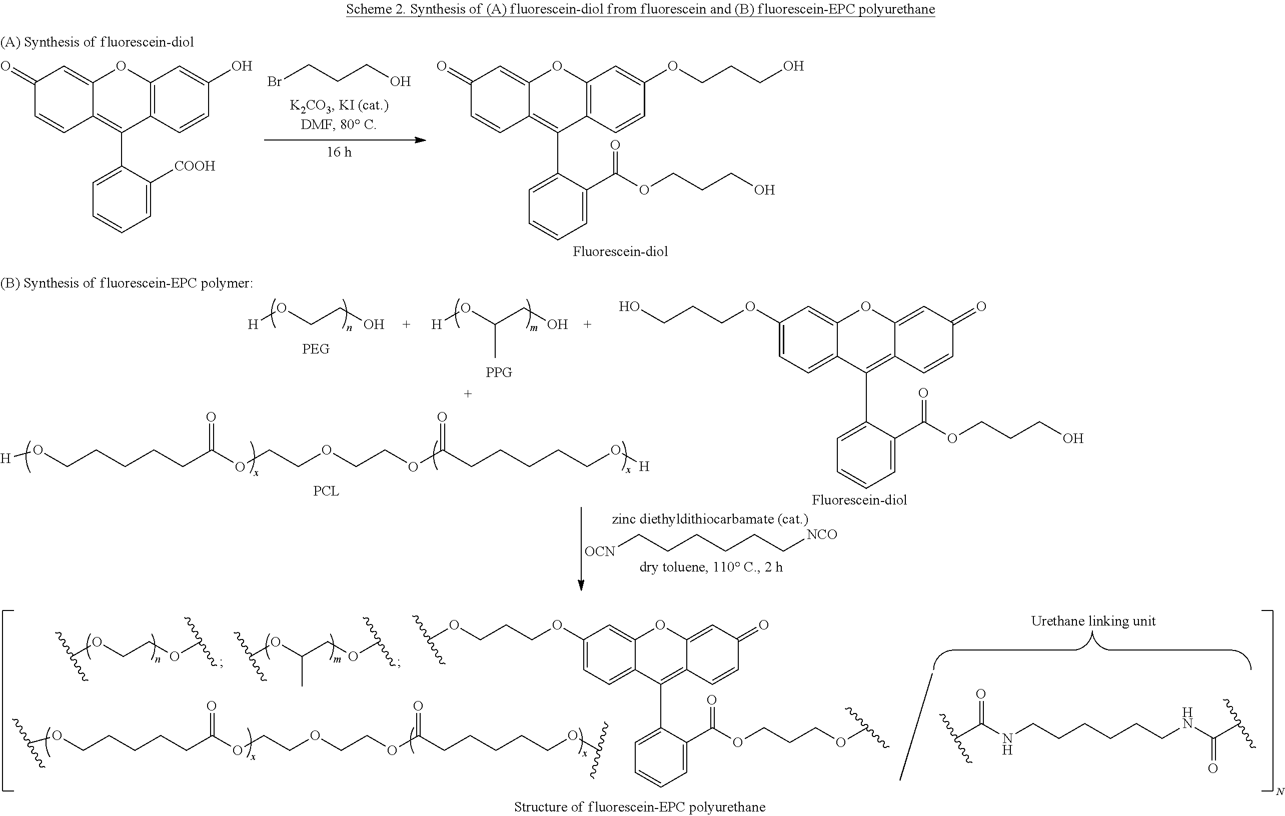

TABLE 1
Summary table showing molecular
weight details of EPC copolymer.

| Number average | Weight average | |
|---|---|---|
| molecular weight, | molecular weight, | Polydispersity |
| Mn (kDa) | Ms (kDa) | index, PDI |
| 55.1 | 83.0 | 1.51 |

TABLE 2
Molar ratios of PEG (E), PPG (P) and PCL (C) incorporated into
each polymer, determined by integration of the ¹H NMR resonances
at 3.60-3.70 ppm, 1.10-1.15 ppm and 4.05 ppm respectively.

| Macromonomer | PEG | PPG | PCL |
|---|---|---|---|
| Mass of (Macro)monomer | 2050 | 2000 | 2000 |
| Mass of repeating unit | 44 | 58 | 114 |
| No of repeating units per macromonomer | 46.2 | 34.2 | 17.4 |
| No of protons per repeating unit for | 4 | 3 | 2 |
| specified NMR peak | | | |
| No of protons per (macro)monomer for | 185 | 103 | 35 |
| specified NMR peak | | | |
| Relative NMR integrals (from spectrum | 302.34 | 38.61 | 1 |
| [a]) | | | |
| Mole ratio of (macro)monomers in | 1.64 | 0.38 | 0.0288 |
| copolymer | | | |
| Normalized mole ratio (PPG set to 1) | 4.35 | 1 | 0.0764 |

10.3. Preparation of nEPCs, F127 Nanomicelles, and nEPCs or F127 Loaded with Rhodamine-Labelled Aflibercept (nEPCs+Rho-A; F127+Rho-A)

CMC values of EPC and F127 were determined using a dye solubilisation method. In-vitro testing utilised either nEPCs (2 wt %) or nEPC+Rho-A. nEPCs (2 wt %) were prepared by dilution of EPC solution (10 wt %). To prepare nEPCs+Rho-A, aflibercept was chemically conjugated with rhodamine based on the protocol provided by Pierce™ NHS-Rhodamine antibody Labelling Kit. To ensure conjugation, 5× excess amount of rhodamine was used. The unreacted excess amount of rhodamine was then removed using a 50 kDA filter unit. The filtration process was repeated until a clear filtrate was obtained. Rhodamine is a small molecule which passes through the filter and separate from conjugated rhodamine. As aflibercept is a macromolecule with a molecular weight of larger than 50 kDA, rhodamine-conjugated aflibercept will be collected inside the filter. To calculate drug concentration, the standard curve of rhodamine-conjugate concentration against fluorescent intensity (Ex: 552 nm, Em: 575 nm) using a Plate Reader (Infinite M200, Tecan) was obtained.

nEPCs+Rho-A of differing nEPC concentrations (0.05, 0.2, 1, 2 wt %) were prepared by dissolving 10 wt % EPC solution in Rho-A solution (0.5 mg/ml). In-vivo testing utilised nEPCs+A (nEPC 2 wt %, aflibercept 40 mg/ml). nEPCs+A were prepared by diluting 10 wt % EPC solution in aflibercept solution (0.5 mg/ml).

10.4. nEPCs Dissolution and NMR Methods

¹H nuclear magnetic resonance (NMR) spectra were recorded using a JEOL 500 MHz NMR spectrometer (Tokyo, Japan) at room temperature. EPC was dissolved in 0.3 mL of aqueous Eylea buffer, and further diluted with 0.4 mL of D₂O. For the sample containing Aflibercept, an equivalent quantity of EPC was dissolved in the Aflibercept solution in aqueous Eylea buffer and further diluted with 0.4 mL of D₂O. Standard water suppression was performed on these samples, and spectra were analysed using the MestReNova software (version 12.0.4) taking reference to the solvent residual peak at 4.66 ppm.

10.5. Transmission Electron Microscopy Characterization of nEPC Morphology

EPC copolymer was dissolved at 1 wt % in either water or in aflibercept solution (0.05 wt %) to form blank nEPCs and nEPCs+A solutions respectively. Samples were prepared on 400 MESH formvar-carbon EM grids (TeddPella01754-F), negatively stained with 2% Uranyl Acetate and air-dried. Grids were analysed in TALOS 120c G2 Transmission Electron Microscope (ThermoFisher Scientific, Massachusetts, USA) operating at 120 kV. Images were collected with CETA-16M camera at 120,000 magnification.

10.6. Characterization of nEPCs and Interaction with Aflibercept

Mean hydrodynamic nanomicellar size of nEPCs (500 μL, 0.2 wt % to avoid particle aggregation) and aflibercept (1000 μL, 0.5 mg/mL) were individually measured by dynamic light scattering (DLS, Zetasizer Nano—Malvern Panalytical, UK) at 25° C., pH 7.2. Aflibercept and nEPC solutions were then mixed and hydrodynamic size was measured to monitor for aflibercept encapsulation. The average values of three micellar diameter measurements of 12 runs were calculated for all samples.

Fluorescence intensity was used to determine EE of nEPCs+Rho-A. Briefly, Rho-A (32 μL of 1000 ng/mL) was added into EPC solution (288 μL) with varied wt % and homogenized. After homogenization, this solution was kept at room temperature for one hour before undergoing filtration with 100 kDA ultra centrifugal filters (Amicon®) to collect free-Rho-A at the bottom of the centrifuge tube. Solution containing free Rho-A was then transferred to spectrophotometer (Ex/Em: 520±20/590±20 nm) for reading. EE was calculated using the following:

$$EE ( \% ) = \frac{( Initial Amount of Rho - A - Final Free Rho - A )}{Initial Amount of Rho - A} \times 100 \%$$

### 10.7. Cell Lines and Mediums

Human Umbilical Vein Endothelial Cells (HUVECs, C2519A) were obtained from Lonza (Basel, Switzerland) and maintained in 25-T flasks in EGM™-2 Endothelial Cell Growth Medium-2 (EGM, CC-3162). Human Corneal Epithelial cells (hCECs, ATCC© PCS-700-010™) were obtained from ATCC (Manassas, Virginia) and maintained in T-25 flasks in corneal epithelial cell growth medium (ATCC® PCS-700-040™). Immortalised adult retinal pigmented epithelial cells (ARPE-19 cells, ATCC® CRL-2302) were obtained from ATCC (Manassas, Virginia) and maintained in Dulbecco's Modified Eagle Medium (DMEM)-F12 (1:1) supplemented with Foetal Bovine Serum (FBS, 10%) and Penicillin-Streptomycin (1%).

### 10.8. In-Vitro Anti-Angiogenic Assays

For the HUVECs proliferation assay, HUVECs (passage 5) were seeded at a density of 15 000 cells/well in EGM on 24-well plate. After overnight culture, cell starvation was conducted by replacing EGM with Endothelial Basal Medium (EBM)+2% Foetal Bovine Serum (FBS) for 6 hours before replacing with appropriate medium based on the 4 experimental arms: CTR (EGM), VEGF (50 ng/mL VEGF165), aflibercept+VEGF (50 ng/mL VEGF165+50 μg/mL aflibercept) and nEPCs+VEGF (50 ng/mL VEGF165+2 wt % nEPCs). Cell proliferation and death were evaluated after 24 and 72 hours by the LDH assay as per instructions provided by the kit.

For the HUVEC migration assay, HUVECs (passage 5) were seeded at a density of 20 000 cells/well in a 96-well plate. Once confluent, a scratch wound was created in each well with the WoundMaker™ (ESSEN Bioscience 4379, UK). Medium (100 μL) based on the above 4 experimental arms were administered to respective wells. Phase contrast images were taken per well, per time point, at the same location using the live cell analysis system (IncuCyte ZOOM®, Sartorius). Images were analyzed with MATLAB (MathWorks; version R2019a) using a method involving frequency filtering and mathematical morphology to approximate the boundaries of cellular regions, adapted from an algorithm according to C. C. Reyes-Aldasoro, D. Biram, G. M. Tozer, C. Kanthou, Electronics Letters 2008, 44, 791. Wound recovery (%) was calculated using [(Aₜ₌₀ₕ−A_{t=Δh})/Aₜ₌₀ₕ]×100, where Aₜ₌₀ₕ is the wound area measured immediately after scratching (time zero), and A_{t=Δh} is the wound area measured at selected time points after the scratch.

For the HUVEC tube formation assay, starved-HUVECs (passage 5) were seeded on Matrigel (Corning® Matrigel® growth factor reduced) coated 96-well plates, at a density of 20,000 cells/well. Solutions (100 uL) based on the above 4 experimental arms were administered to respective wells. Bright field images were taken 5 hours after exposure. Tube formation was analysed using the Angiogenesis Analyzer in the ImageJ software.

The in-vitro anti-angiogenic assay was performed using an AIM 3D chip (AIM Biotech, Singapore), according to the AIM Biotech protocol with type I collagen solution (2 mg/mL) in the middle channel of the device. The left and right microchannels were then coated with fibronectin. The left fibronectin-coated lateral fluidic channel was then seeded with HUVECs in EGM at a density of 3×10⁶ cells/mL. After 24 hours, human VEGF165 (40 ng/mL) and Sphinogosine-1-phosphate (S1P) (125 nM) in EGM were added to both the left and right channels. This was used as a positive control. Three groups, aflibercept (50 μg/mL), nEPCs (2 wt %) and nEPCs+A, were then prepared with VEGF and S1P and applied to the cell-channel and the right-lateral fluidic channel. The AIM Chips (FIG. 19) were maintained at 37° C. and 5% CO₂ for 3 days with daily medium change. Angiogenic sprouting of HUVECs in the collagen hydrogel was monitored daily with a phase-contrast microscope. Confocal imaging was performed on the 5th day with a laser scanning microscope (Carl Zeiss LSM800 scanhead on a Imager.Z2 microscope controlled by Zen 2.1), using Plan-Apochromat 10×/0.45-NA objective.

### 10.9. HUVEC RNA Expression Analysis

HUVECs RNA expression was determined via qPCR. HUVECs were seeded at 100 000 cells/well in a 24-well plate. After 24 hours, 400μL solutions based on the 4 experimental arms (Buffer, VEGF, aflibercept+VEGF, nEPCs+VEGF)) were added. After 24 hours of incubation, the RNeasy Mini Kit (Qiagen, GmbH, Hilden, Germany) was used to extract and purify the total RNA, which was converted to cDNA using iScript Reverse Transcription Supermix for RT-qPCR (Bio-Rad Laboratories Inc, USA). The reaction mixture was topped with RNAse-free water to 20 μL before undergoing synthesis in the thermal cycler. cDNA was then dilated to be used for real-time PCR analysis. Each real-time PCR reaction included 2 μL of diluted cDNA solution, RNAse-free water, respective forward and reverse primer mix (10 μM) and SYBR Green real-time PCR mix. Reactions were carried out in triplicates in a real time PCR system (Applied BioSystems QuantStudio 5). GAPDH was used as an internal control every single reaction plate.

### 10.10. Cytotoxicity Studies

Cytotoxic effects of nEPCs (0.01-2 wt %) on hCECs and ARPE-19 were evaluated using LDH proliferation and cell leakage assays (Cytotoxicity LDH Assay Kit, Dojindo, DOJD-CK12, Japan) according to the manufacturer's protocol. In brief, cells were seeded at a density of 10 k/well in 96-well plates. After overnight culture, the cells were exposed to 100 μL of nEPC solutions (0, 0.01, 0.02, 0.05, 0.1, 0.2, 0.5, 1 and 2 wt %) for 24 hours. For LDH proliferation assay, all groups were lysed and absorbance was measured at 490 nm and subtracted from 650 nm. Proliferation (%)=100%×(A/B). For test groups, supernatant was collected. For positive control, cells were lysed before collecting the supernatant. Absorbance for all groups was measured as per previously stated. Cell death (%)=100%×[(A−C)/(B−C)]. Identical cytotoxicity studies were also done for HUVECs (FIG. 8 and FIG. 9).

### 10.11. Trans Epithelial Electrical Resistance in Porcine Tissue

For porcine corneal tissue, the tissue integrity was monitored by an Ussing Chamber (WPI, U.S). Briefly, the freshly excised porcine cornea was gently mounted in a sample clip, and then were inserted vertically between the two halves of Ussing. The donor (corneal epithelium side) and the receiver (corneal endothelium side) were each filled with 7.5 mL Glutathione bicarbonate Ringer's (GBR) solution, and were continuously aerated with gas mixture of Carbogen (95% O2 and 5% CO2) to maintain the activity of cornea. The corneas were stimulated by a continuous electric current pulse of 10 mV for 0.2 s every 1 min, and real-time monitoring for the electrical parameters of cornea was controlled by LabChart7 software. When the electric parameters were basically stable, the total liquid in both sides were removed. Immediately, 0.2 mL of sample solution was added to donor chamber, and 0.4 mL of GBR was added into receiving chamber at 37° C. At pre-fixed intervals of 10 min, 0.2 mL of PBS was taken from the receiver chamber and equal volume of GBR solution was supplemented. The sample collection was continued to 40 min. At the end of penetration, GBR was refilled into the chamber, the tissue integrity was checked and monitored for another 30 min. The penetrated Rho-Aflibercept was analysed by microplate reader to detect the fluorescence intensity.

### 10.12. Ex-Vivo Anti-Angiogenic Choroidal Assays

Choroidal sprouting assay was performed according to a published protocol in Shao, Z. et al., PLoS One 2013, 8, e69552, the contents of which are fully incorporated herein by reference. In brief, murine choroidal tissue was cut into small pieces and embedded in Matrigel. Explants were cultured ex-vivo in EGM+5% FBS on a 24-well plate. After 48 hours of incubation at 37° C., explants were monitored for blood vessel growth. The medium of each well was replaced by appropriate medium (500 uL) based on the 3 experimental groups: VEGF (EGM+5% FBS+50 ng/mL VEGF165), aflibercept+VEGF (EGM+5% FBS+50 ng/mL VEGF165+1 mg/mL aflibercept), nEPCs+VEGF (EGM+5% FBS+50 ng/mL VEGF165+2 wt % nEPCs). After 2 days, explants were monitored and images were acquired with a bright-field microscope (Nikon Eclipse Ti using a Plan UW 2×/0.06-NA objective).

For the choroidal regression assay, the explants were cultured in in EGM+5% FBS over a 4-day duration. Once vessel sprouting was established, the medium of each well was replaced with the same experimental groups used in sprouting assay. On Day 3 and 4, explants were monitored. Sprouting area was quantified by the TRI2 software and normalised to the explant size based on the published protocol according to Shao, Z. et al., PLoS One 2013, 8, e69552, the contents of which are fully incorporated herein by reference. ImageJ 1.46r (NIH) was used to analyse the phase contrast images of the choroid sprouts. The choroidal tissue in the centre of the sprouts was outlined and removed by adjusting the wand tool to 30%. The threshold function was used to define the microvascular sprouts against the background and periphery. The total number of threshold-outlined pixels were then calculated for quantification. For the inhibition assay, the area of sprouting was directly measured in pixel units. To correct for the difference in initial explant size, the sprouting area was calculated using the following:

$$Sprounting Area ( \% of original ) = \frac{( Final sprouted area - initial sprouted area )}{Total Initial Area} \times 100 \%$$

10.13. In-Vitro Assessment of Rho-A and nEPCs+Rho-A Uptake by hCECs

hCECs were seeded on 24-well plate on a gelatin-coated glass slide at a density of 10,000 cells/well in corneal epithelial growth medium (500 μL). After culturing at 37° C. and 5% CO₂ humidified atmosphere for 24 hours, the medium was replaced with growth medium containing Rho-A (0.5 mg/mL) with varying nEPC solutions (0, 0.05, 0.2, 1, 2 wt %). The cells underwent PBS wash 4 times to remove any excess extracellular aflibercept. Finally, the cells were observed under a confocal laser scanning microscope (Carl Zeiss LSM800 scanhead on a Imager.Z2 microscope controlled by Zen 2.1). Images were acquired using Plan-Apochromat 100×/1.4-NA oil DIC objective for slides. Laser lines on the system were 405 nm, 488 nm, 561 nm, and 640 nm.

10.14. Quantitative In-Vitro Cellular Uptake in hCECs by FACTs

hCECs were seeded at a density of 60,000 cells/well on a 12-well plate. The cells were cultured and exposed to the same medium used for internalization. After 24 hours of exposure, samples were aspirated and cells were carefully washed four times with PBS and trypsinized. The cells were suspended in growth medium (400 μL), the internalized Rho-A (0.5 mg/mL) or nEPCs+Rho-A (Rho-A: 0.5 mg/mL, nEPCs: 2 w.t %) was quantified by flow cytometry (BD Bioscience FACS Aria II, United States), excited at wavelengths of 564 nm and monitored at wavelengths of 590 nm. The results were analysed by the FlowJo Software. All the evaluation for F127 was strictly follow the same conditions.

### 10.15. Topical Permeability Ex-Vivo Model of Porcine Cornea

The topical penetration of nEPCs+Rho-A (nEPC: 2 wt %, Rho-A: 0.5 mg/ml) was evaluated using an ex-vivo model of porcine cornea. Adult porcine eyes were obtained within 3 hours of the animal's death. Eyes were irrigated with PBS and a drop of Rho-A (20 uL, 40 mg/mL) and nEPCs+Rho-A was administered on the cornea directly. The eyes were washed with PBS after 40 minutes of incubation at 37° C. The vitreous was then harvested to determine the fluorescence intensity emitted from Rho-A. This was done using a plate reader (ex: 560 nm/em: 594 nm)

The permeability of the Rho-A (40 mg/mL) and nEPCs+Rho-A through the porcine sclera was evaluated using a vertical Ussing electrode kit (World Precision Instruments, Florida, U.S). The sclera was placed vertically between the diffusion cells with epithelium oriented to the donor cells. The setup was maintained at 37° C. The donor cell contained Rho-A (0.2 mL) and nEPCs+Rho-A solution while the acceptor chamber had PBS (0.4 mL). The PBS was collected every 10 minutes and the chamber was replenished with another PBS (0.4 mL). The experiment was stopped at 40 minutes and the concentration of penetrated aflibercept was calculated based on the fluorescence intensity (Sample size 3).

### 10.16. Animal Studies

Male wild-type C57B/6J mice, ranging 6 to 8 weeks old, were obtained from In Vivos (Singapore) and used for all in-vivo experiments. All animal procedures were conducted in accordance with the ARVO Statement for The Use of Animals in Ophthalmic and Vision Research. The experiment was approved by the A*STAR Institutional Animal Care and Use Committee (IACUC): #191 488 for project titled: Testing of therapeutic agents for ocular delivery of drugs.

### 10.17. In-Vivo Assessment of Corneal Retention Time

To assess corneal retention time of nanomicelles complexed Rho-A (nEPCs+Rho-A: 2 wt %, Rho-A: 0.5 mg/ml) and Rho-A (5 μL, 40 mg/mL), a single eyedrop of each was administered to separate murine cornea surfaces. Manual blinking of the murine eye was performed every 15 seconds. Anterior segment optical coherence tomography (OPTOVUE, RTVue) was performed 30, 60, 120, 210, 285, 300 seconds after initial eyedrop. For each ASOCT image, the area above the corneal surface which was occupied by the eyedrop was selected and quantified using ImageJ.

### 10.18. In-Vivo Aflibercept Cornea Penetration Studies

To assess topical permeability of nanomicelles complexed Rho-A (nEPCs or F127: 2 wt %, Rho-A: 0.5 mg/ml), a single eye drop of Rho-A (5 μL, 40 mg/mL) or nEPCs+Rho-A (F127+Rho-A) was administered to the murine cornea surface. Mice were sacrificed and enucleated 40 minutes after eyedrop application. Eyes were then embedded in OCT compound (Sakura Finetek, USA) followed by 4% paraformaldehyde fixation before making 10 μM thick cryosections for observation under a confocal laser scanning microscope (Olympus FV1000 Confocal head on IX81 microscope controlled by Fluoview 4.2).

Penetration of Rho-A into the vitreous cavity was assessed by obtaining a sample of vitreous humour. Limbal puncture was made with a 30-gauge needle and vitreous was extracted with a thin glass capillary tube. Vitreous humour from 10 eyes were pooled within each group to assess the amount of aflibercept using a spectrophotometer (ex: 560±20 nm/em: 590±20 nm).

10.19. In-Vivo Bioactivity Assessment of nEPCs+A

A mouse model of laser-induced CNV, as previously published in Nirmal, J. et al., Exp Eye Res 2020, 199, 108187, the contents of which are fully incorporated herein by reference, was used to assess the bioactivity of nEPCs+A. Mice were anaesthetized using intraperitoneal ketamine (150 mg/kg) & xylazine (10 mg/kg). In these eyes, photocoagulation was induced using an image guided laser system (Micron IV, Phoenix Research Laboratories, Pleasanton, CA). The mice were divided into 4 arms of 8 eyes each, including: Buffer (PBS); aflibercept (40 mg/mL); nEPC (2 wt %) and nEPCs+A (aflibercept 40 mg/mL, nEPC 2 wt %). Each solution was applied immediately after laser treatment, 3 times daily, with 1-hour intervals, for 14 days.

Mice were anaesthetised as above prior to fundus fluorescein angiography (FFA) using the retinal imaging system (Micron IV, Phoenix Research Laboratories) at Days 3, 7 and 14 after laser photocoagulation. FFA images were taken at 5 and 10 minutes after fluorescein injection.

The mice were euthanized and enucleated 14 days after laser for preparation of choroidal flat mount. Eyes were fixed in 4% paraformaldehyde in PBS overnight at 4° C. The anterior segment and retina were embedded in paraffin for immunostaining. The eyecups were incubated with isolectin B4 at 4° C. for choroidal vessel staining before 3 cycles of PBS wash. After making four incisions radial to the optic nerve, the tissue was flat-mounted, and Z-stack images of the CNV lesions were taken with the confocal microscope (LSM700, Zeiss, Thornwood, NY).

The angiograms and Z-stack images were imported into ImageJ (US National Institutes of Health, Bethesda, MD, USA). The maximal border of the CNV lesion on each image was manually delineated under magnification, with the area quantified as the number of pixels per 100 μm. The fluorescence intensity of the CNV lesions was graded using ImageJ (National Institutes of Health, Bethesda, MD) by 2 independent graders with single blinding. The rate of CNV regression was calculated using the following:

$$Rate of CNV Regression = \frac{Leakage area on 3 rd day - leakage area on 14 th day}{14 - 3 days}$$

### 10.20. Statistical Analysis

All data were reported as mean±s.d. Statistically significant differences between samples were determined by one-way analysis of variance (ANOVA) followed by pairwise testing with Tukey's honest significance difference (HSD) post-hoc test. P-values below 0.05 on a 2-tailed test were considered significant (*p<0.05, **p<0.002 and ***p<0.0002, ****p<0.0001). All analyses were performed using GraphPad Prism (ver. 8.1.1).

### 10.21. Material Characterisation

NMR spectra were recorded at room temperature on a JEOL ECA 500 MHz NMR spectrometer operating at 500 MHz, with the samples dissolved in CDCl₃ (NMR solvents purchased from Cambridge Isotopes Laboratory Inc.). Chemical shifts were reported in parts per million (ppm) on the 5 scale. Gel permeation chromatography (GPC) analyses were performed on a Waters GPC machine at 40° C., equipped with a 515 HPLC pump, Waters Styragel columns and Waters 2414 refractive index detector. HPLC grade THF was used as the eluent at a flow rate of 1.0 mL/min. Monodisperse polystyrene standards were used to generate the calibration curve.

It will be appreciated by a person skilled in the art that other variations and/or modifications may be made to the embodiments disclosed herein without departing from the spirit or scope of the disclosure as broadly described. For example, in the description herein, features of different exemplary embodiments may be mixed, combined, interchanged, incorporated, adopted, modified, included etc. or the like across different exemplary embodiments. The present embodiments are, therefore, to be considered in all respects to be illustrative and not restrictive.

1. An anti-angiogenic agent comprising:
a multi-block copolymer in the form of one or more micelles,
wherein the copolymer comprises a first poly(alkylene glycol) block, a second poly(alkylene glycol) block and a polyester block.

2. The anti-angiogenic agent as claimed in claim 1, wherein the copolymer comprises at least urethane/carbamate linkage(s) and/or allophanate linkage(s).

3. The anti-angiogenic agent as claimed in claim 1, wherein the molar ratio of the first poly(alkylene glycol) block to the second poly(alkylene glycol) block to the polyester block in the copolymer is about 1 to 10:1:0.01 to 1.5.

4. The anti-angiogenic agent as claimed in claim 1, wherein the first and second poly(alkylene glycol) are selected from the group consisting of poly(ethylene glycol) (PEG), poly(propylene glycol) (PPG), poly(butylene glycol) and combinations thereof, and the polyester is selected from the group consisting of polycaprolactone (PCL), poly(lactic acid) (PLA), poly(lactic-co-glycolic acid) (PLGA), polyhydroxyalkanoate (PHA) and combinations thereof.

5. The anti-angiogenic agent as claimed in claim 1, wherein the total polymer concentration of the copolymer is in the range of from 0.01 wt % to 6 wt %.

6. The anti-angiogenic agent as claimed in claim 1, wherein the anti-angiogenic agent comprises a water content of at least 90 wt %.

7. The anti-angiogenic agent as claimed in claim 1, wherein the one or more micelles have a hydrodynamic size of from 1 nm to 100 nm.

8. The anti-angiogenic agent as claimed in claim 1, wherein the anti-angiogenic agent further comprises one or more bioactive(s) complexed with or encapsulated by the copolymer micelles.

9. The anti-angiogenic agent as claimed in claim 8, wherein the one or more bioactive(s) comprises an anti-vascular endothelial growth factor (anti-VEGF), optionally wherein the anti-VEGF is selected from the group consisting of bevacizumab, aflibercept, ranibizumab and brolucizumab.

10. (canceled)

11. The anti-angiogenic agent as claimed in claim 8, wherein the one or more bioactive(s) is encapsulated by the copolymer micelles at an encapsulation efficiency of more than 25%.

12. The anti-angiogenic agent as claimed in claim 1, wherein the anti-angiogenic agent is formulated as a topical ophthalmic formulation.

13. A method of preparing anti-angiogenic agent as claimed in claim 1, the method comprising:
adding a copolymer to an aqueous medium at a concentration that is no less than the critical micelle concentration of the copolymer but no more than the sol-gel transition concentration of the copolymer, to form micelles,
wherein the copolymer comprises a first poly(alkylene glycol) block, a second poly(alkylene glycol) block and a polyester block.

14. The method as claimed in claim 13, wherein the concentration of the copolymer in the aqueous medium is in the range of from 0.01 wt % to 6 wt %.

15. The method as claimed in claim 13, further comprising complexing or encapsulating one or more bioactive(s) with the micelle.

16. The method as claimed in claim 13, further comprising coupling the first poly(alkylene glycol) block, the second poly(alkylene glycol) block and the polyester block together by at least urethane/carbamate linkage(s) and/or allophanate linkage(s), optionally wherein the first and second poly(alkylene glycol) are selected from the group consisting of poly(ethylene glycol) (PEG), poly(propylene glycol) (PPG), poly(butylene glycol) and combinations thereof; and the polyester is selected from the group consisting of polycaprolactone (PCL), poly(lactic acid) (PLA), poly(lactic-co-glycolic acid) (PLGA), polyhydroxyalkanoate (PHA) and combinations thereof.

17. (canceled)

18. The method as claimed in claim 13, wherein the coupling step is carried out in the presence of a coupling agent comprising an isocyanate monomer that contains two isocyanate functional groups,
and/or the coupling step is carried out in the presence of a catalyst selected from the group consisting of alkyltin compounds, aryltin compounds and dialkyltin diesters such as dibutyltin dilaurate, dibutyltin diacetate, dibutyltin dioctanoate and dibutyltin distearate,
and/or the coupling step is carried out in the presence of a solvent selected from the group consisting of toluene, benzene, xylene, halogenated organic solvents, halogenated alkane solvents, chlorinated solvents, dichloromethane, dichloroethane, tetrachloromethane and chloroform (or trichloromethane).

19.-25. (canceled)

26. A method of preventing or treating an eye disorder and/or cancer, the method comprising administering the anti-angiogenic agent as claimed in claim 1 to a subject in need thereof.

27. (canceled)

28. The method of claim 26, wherein the eye disorder is selected from the group consisting of angiogenic eye disorders, ocular diseases in the anterior segment, ocular diseases in the posterior segment, neovascular related ophthalmic posterior segment diseases, retinal diseases, neovascular age-related macular degeneration (AMD) such as neovascular AMD, diabetic retinopathies, diabetic macular oedema (DMO), choroidal neovascularisation (CNV), central retinal vein occlusion (CRVO), corneal neovascularization, and retinal neovascularization.

29. The method of claim 26, wherein the anti-angiogenic agent is to be topically administered to a subject in need thereof.

30. The method of claim 26, wherein the anti-angiogenic agent is formulated as an eye drop.
